(19) United States
(12) Patent Application Publication
(10) Pub. No.: US 2021/0023267 A1
(43) Pub. Date: Jan. 28, 2021

(54) POLYMER-PERMEATED GRAFTS AND METHODS OF MAKING AND USING THE SAME

(71) Applicant: BIOAESTHETICS CORPORATION, Research Triangle Park, NC (US)

(72) Inventors: Nicolas PASHOS, Durham, NC (US); William HEIM, Durham, NC (US)

(21) Appl. No.: 17/044,942

(22) PCT Filed: Apr. 2, 2019
(86) PCT No.: PCT/US2019/025306

§ 371 Date: Oct. 2, 2020

Related U.S. Application Data
(60) Provisional application No. 62/651,485, filed on Apr. 2, 2018.
(60) Provisional application No. 62/775,963, filed on Dec. 6, 2018.

(51) Int. Cl. A61L 27/36; A61L 27/24; A61L 27/54; A61L 27/38; A61L 27/20; A61L 27/16

(52) U.S. Cl. CPC A61L 27/362; A61L 27/24; A61L 27/54; A61L 27/3625; A61L 27/3633; C08L 33/18; A61L 27/20; A61L 27/16; A61L 2300/406; A61L 2400/18; A61L 27/38

(57) ABSTRACT

This invention is directed to polymer-permeated grafts and methods of making and using the same.

This application claims priority from U.S. Provisional Patent Application No. 62/651,485, filed on Apr. 2, 2018, and 62/775,963, filed on Dec. 6, 2018, the contents of which are each incorporated by reference in their entireties.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to polymer-permeated grafts and methods of making and using the same.

### BACKGROUND OF THE INVENTION

Plastination is a technique which uses polymers to permit the preservation of bodies, body parts, anatomical specimens and surgical specimens in a physical state approaching that of the living condition. There are limitations and disadvantages of plastination that prevents it from being used in a clinical setting. Further, the plastinates that are obtained after the plastination procedure are generally used in a non-clinical setting, such as for the preservation of tissues for teaching purposes or histological examination. Aspects of the invention address these unmet needs, providing resin-impregnated grafts for in vivo use and methods of making and using the same.

### SUMMARY OF THE INVENTION

The present invention provides a polymer-permeated graft for in vivo use in a subject.

In embodiments, the polymer-permeated graft comprises a tissue substantially free of cells and which is permeated with a polymer that is substantially uniformly distributed in the tissue. In embodiments, the decellularized tissue is substantially free of water or is free of water.

In embodiments, the graft comprises less than about 50% polymer. In embodiments, the graft comprises more than 0% polymer, more than about 0.1% polymer, more than about 1% polymer, more than about 5% polymer, more than about 10% polymer, more than about 20% polymer, more than about 30% polymer, more than about 40% polymer, more than about 50% polymer.

In embodiments, the tissue comprises a dermal tissue and/or an epidermal tissue.

In embodiments, the tissue comprises an organ (for example a lung or a liver), a muscle, a ligament, a bone, a nipple, areola, a nipple attached to an areola, a lip, skin, a tendon, an aorta, a blood vessel.

In embodiments, the decellularized tissue substantially retains at least one matrix molecule. For example, the matrix molecule is a component of the extracellular matrix, non-limiting examples of which comprise laminin, elastin, fibronectin, collagen (such as a Type I collagen, a Type III collagen, a Type IV collagen, a Type VI collagen, or a combination thereof), or a combination thereof.

In embodiments, the decellularized tissue is substantially free of skin, fat and/or fibrous tissue.

In embodiments, the polymer-permeated graft comprises a natural polymer (such as alginate or collagen) and/or synthetic polymer (such as cyanoacrylate).

In embodiments, the polymer comprises a colored polymer, such as a polymer comprising melanin, a dye, or a combination thereof.

In embodiments, the polymer comprises at least one viable cell, a polymer comprising at least one antibiotic, a biodegradable polymer (for example, chitosan, collagen, alginate, cyanoacrylate, dermabond), a non-biodegradable polymer (for example, silicon, UHMWPE), a polymer capable of cross-linking, or a combination thereof.

In embodiments, the cells have been introduced into polymer-permeated graft under conditions conducive to repopulate the tissue with the cells or progeny thereof. For example, the viable cells comprise exogenous cells, autologous cells, allogenic cells. Non-limiting examples of cell types utilized comprise stromal cells, fibroblasts, endothelial cells, progenitor cells, stem cells, organ-specific cells, tissue-specific cells, keratinocytes, melanocytes, a nerve cell, or a combination thereof.

The present invention further provides a method of making a polymer-permeated graft for in vivo use.

In embodiments, the method comprises obtaining a decellularized tissue; optionally, fixing the decellularized tissue by submerging the decellularized tissue in a fixative for a period of time sufficient to fix the tissue; replacing substantially all of the water within the tissue with a solvent by submerging the decellularized tissue in the solvent for a period of time and at a temperature sufficient to replace all or substantially all of the water within the tissue; optionally, removing all or substantially all of the fat within the tissue by submerging the decellularized tissue in a solvent for a period of time and at a temperature sufficient to remove substantially all lipids; permeating the tissue with a polymer by submerging the tissue in the polymer and subjecting the submerged tissue to vacuum for a period of time sufficient to permeate the tissue with the polymer; optionally, cross-linking the polymer permeated within the tissue; wherein the polymer-permeated tissue comprises the polymer substantially uniformly distributed in said tissue; thereby providing a polymer-permeated graft for in vivo use.

In embodiments, the chemical cross-linker can be admixed with the polymer or precursor thereof prior to permeating the decellularized tissue and/or matrix.

Embodiments can further comprise decellularizing a tissue of cells of the epidermis and/or cells of the dermis, while substantially retaining at least one matrix molecule, such as a molecule of the extracellular matrix.

For example, the matrix molecule is a component of the extracellular matrix, non-limiting examples of which comprise laminin, elastin, fibronectin, collagen (such as a Type I collagen, a Type III collagen, a Type IV collagen, a Type VI collagen, or a combination thereof), or a combination thereof.

Embodiments can still further comprise repopulating the tissue with viable cells under conditions conducive to repopulate the tissue with the cells or progeny thereof. The repopulating occurs at about the same time as the permeating step, and/or the repopulating occurs after the permeating step. The viable cells can comprise exogenous cells, autologous cells, allogenic cells. Non-limiting examples of cell types utilized comprise stromal cells, fibroblasts, endothelial cells, progenitor cells, stem cells, organ-specific cells, tissue-specific cells, keratinocytes, melanocytes, a nerve cell, or a combination thereof.

In embodiments, the fixative comprises glutaraldehyde, genipin.

In embodiments, the solvent comprises acetone, xylene, alcohol.

In embodiments, the decellularized tissue is incubated in acetone at about −15° C. to 25° C. for a period of time.

In embodiments, the cross-linking comprises UV cross-linking, chemical cross-linking.

Still further, the invention is directed towards method of grafting to a subject a polymer-impregnated graft.

In embodiments, the method comprises obtaining the polymer-permeated graft as described herein and implanting the polymer-permeated graft to a site on the subject; thereby grafting to a subject the polymer-permeated graft.

In embodiments, the polymer-permeated graft has been repopulated with viable cells. The viable cells can comprise exogenous cells, autologous cells, allogenic cells. Non-limiting examples of cell types utilized comprise stromal cells, fibroblasts, endothelial cells, progenitor cells, stem cells, organ-specific cells, tissue-specific cells, keratinocytes, melanocytes, a nerve cell, or a combination thereof.

Other objects and advantages of this invention will become readily apparent from the ensuing description.

### BRIEF DESCRIPTION OF THE FIGURES

FIG. 1 shows a schematic of a method of making a polymer-permeated graft. (NAC: nipple areola complex).

FIG. 2 shows a schematic of protein fibers and polymer structure.

FIG. 3 shows examples of applications of a polymer-permeated graft.

FIG. 4 are cryo-scanning electron micrographs of intact and acellular skin from rhesus macaque showing collagen bundles and fibers in the epidermis and dermis.

FIG. 5 is a schematic of the plastination process of the invention. (Top) An acetone soaked ABG is incubated in a polymer bath (purple) and subjected to vacuum force impregnation causing polymer to replace escaping acetone. ABG is washed and placed under bath conditions (polymer-dependent) to induce polymerization. (Bottom) Magnified view of ECM graft being vacuum force impregnated with monomers before crosslinking of monomers to polymers within interstitial space.

FIG. 6 is a schematic showing intact human skin that is decellularized and cut to size. Polymers are biotinylated and vacuum impregnated into acellular skin grafts using different combinations of polymer, concentration, and impregnation time. Impregnated polymers are then measured for polymer occupancy and distribution. Successful grafts are then measured for mechanical strength and bioactivity.

FIG. 7 shows NAC reconstruction

FIG. 8 is a schematic for the decellularization and engraftment process of the NAC for human use. Steps: 1) Removal of a cadaveric NAC for graft generation or removal of patient NAC as part of mastectomy. 2) Decellularization of donor NAC. 3) Acellular NAC graft onlay engrafted onto patient 4) Natural repopulation of acellular NAC scaffold with patient's cells, resulting in regeneration of NAC.

FIG. 9 shows histology of representative dcl-NHP NAC. (Far left) H&E micrograph of a randomly selected dcl-NHP NAC. Regions in yellow boxes are shown in greater magnification in micrographs 1-3.

FIG. 10 shows weight and neovascularization (murine). For all results One-way ANOVA with Tukey's posthoc test was performed. *=p<0.05; **=p<0.01; ***=p<0.001.

FIG. 11 shows histology of representative dcl-NHP NAC (murine model). (below) H&E micrograph of a randomly selected dcl-NHP NAC. Region in green box are shown in greater magnification in micrographs 1-3.

FIG. 12 shows body weights of NHP. A) Body weights of NHP. B) Graph of averaged weights from periods of study.

FIG. 13 shows erythrocytes. A) Graph of erythrocyte cell counts. B) Graph of averaged erythrocyte cell counts from periods of study.

FIG. 14 shows platelet counts. A) Graph of platelet counts from before, during, and after the engraftment experiment. B) Graph of averaged platelet counts from periods of study.

FIG. 15 shows erythrocyte properties. A) erythrocyte properties B) Graph of averaged erythrocyteproperty values from periods oHgb (hemoglobin), Hct (hematocrit), Mcv (mean corpuscular volume), Mch (mean corpuscular hemoglobin), Mchc (mean corpuscular hemoglobin per cell), and Rdw (red blood cell distribution width).

FIG. 16 shows leukocyte profile from NHP peripheral blood. A) Graph of white blood cells from before, during, and after the engraftment experiment B) Graph of averaged leukocyte cell counts from periods of study. WBC (white blood cell), Neu (neutrophil), Lym (lymphocyte), Mon (monocyte), Eos (eosinophil), and Bas (basophil).

FIG. 17 shows electrolytes. A) Graph of electrolytes from before, during, and after the engraftment experiment. B) Graph of averaged electrolyte levels from periods of study. For all results One-way ANOVA with Tukey's posthoc test was performed. *=p<0.05; **=p<0.01; ***=p<0.001.

FIG. 18 shows histology of representative dcl-NHP NAC (below) A) decellularized NHP NAC B) Native vs Decell, C) engraftment orientation, D) Week 1 vs week 6 H&E. E) magnification of center of week 6 from part D (green square).

FIG. 19 is an illustration showing one process to generate drug loaded polyABG. Donor derived human skin is decellularized using our patent-pending process and cut to size. Grafts are saturated in an organic solvent, submerged in a polymer and drug bath mixture, and subject to vacuum pressure. The high vapor pressure of the organic solvent causes escape from the graft, creating a change in pressure within the interstitium. Upon return to normal pressure, the surrounding polymer and drug mixture is force-impregnated into the graft. The drug loaded polyABG is washed and placed under conditions to allow polymerization of polymer into a hydrogel which physically entraps the drug.

FIG. 20 is a table showing drug+polyABG embodiments described herein compared to current clinical products.

FIG. 21 shows data for dermal polyABGs. Dehydrated, acetone-saturated acellular human skin (1×1×0.4 cm) was incubated in the presence of gelatin or silk fibroin solutions under identical conditions, with only pressure varying. “Impregnation”: vacuum applied. “Diffusion”: atmospheric pressure. “Control”: dehydrated and acetonesaturated acellular human skin in the presence of carrier solution (PBS). (Top) H&E stains of gelatin-impregnated acellular human skin. Gelatin appears as light pink stain within the interstitium. (Bottom) Alcian blue stains of silk fibroin-impregnated acellular human skin. Silk fibroin appears as dark blue stain within the interstitium. Images shown are from the center of the graft at a ˜2 mm depth from the graft surface. Hydrogel formation was induced by exposing polyABG to chilled PBS bath for gelatin and methanol for silk fibroin.

FIG. 22 shows cryo-scanning electron micrographs of intact and acellular skin from rhesus macaque showing collagen bundles and fibers in the epidermis and dermis.

FIG. 23 shows (Top) an acetone soaked ABG is incubated in a polymer bath (purple) and subjected to vacuum force impregnation, enabling the polymer to replace escaping acetone. The ABG is washed and placed under bath conditions (polymer-dependent) to induce polymerization. (Bottom) Magnified view of ECM graft being vacuum force impregnated with monomers before crosslinking of monomers to polymers within interstitial.

FIG. 24 shows intact human skin is decellularized and cut to size. Polymers are biotinylated and vacuum impregnated into acellular skin grafts using different combinations of polymer, concentration, and impregnation time. Impregnated polymers are then measured for polymer occupancy and distribution. Successful grafts are then measured for mechanical strength and bioactivity.

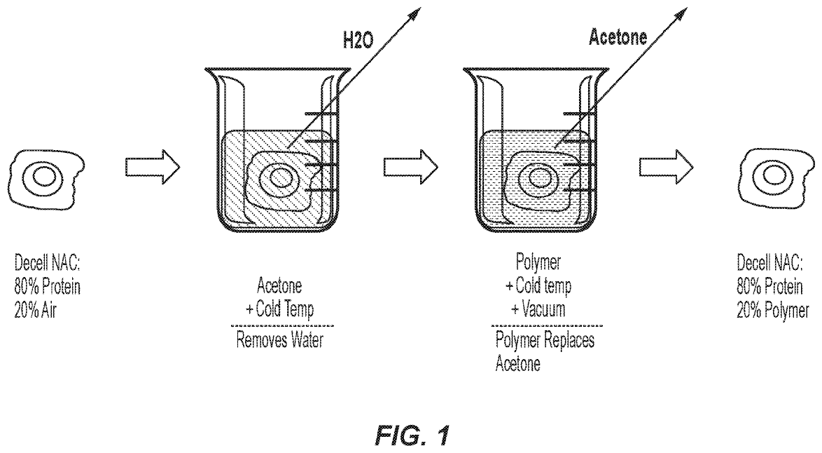

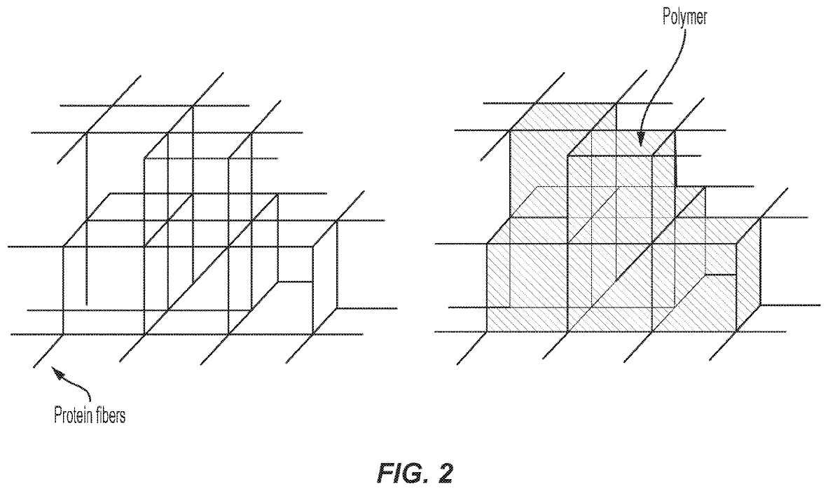

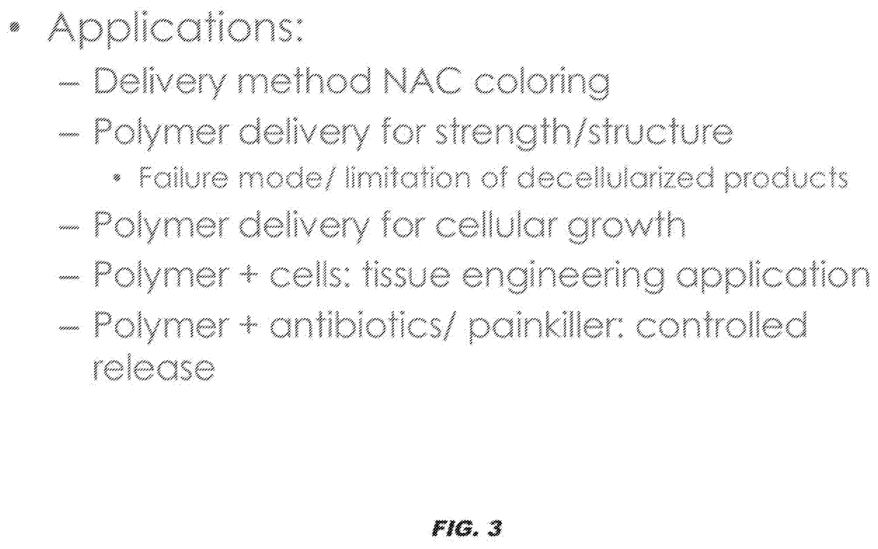

### DETAILED DESCRIPTION OF THE INVENTION

### Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Plastination is a technique which uses polymers to permit the preservation of bodies, body parts, body tissues, anatomical specimens and surgical specimens in a physical state approaching that of the living condition. During the plastination process, a curable polymer is pulled or drawn into the structure of the tissue using a vacuum chamber. This procedure utilizes polymers which are forcefully impregnated into the tissues to make them stable and free from deterioration.

Decellularized Polymer-Permeated Graft

Aspects of the invention are directed towards compositions comprising decellularized polymer permeated grafts, such as those that can be implanted in a subject. A “graft” can refer to a structure or composition that is implanted or attached to an individual to replace an anatomical feature or to correct an anatomical defect.

In embodiments, the grafts described herein can be used for in vivo uses, such as replacing nipples or areolas, or both, that have been surgically removed or have been lost due to trauma. Polymer-permeated grafts described herein can be made from any tissue, structure, or appendage of a subject's body that can be decellularized and/or substantially retains at least one matrix molecule after decellularization, such as an organ (for example a lung or liver), a muscle, a ligament, a bone, a nipple, areola, a nipple attached to an areola, a lip, skin, a tendon, an aorta, a blood vessel. In embodiments, the graft comprises a dermal tissue and/or an epidermal tissue, such as a decellularized dermal tissue and/or epidermal tissue.

The graft can comprise any tissues that substantially retains at least one matrix molecule after the decellularization process. For example, the matrix molecule can comprise a molecule or component of the extracellular matrix. “Extracellular matrix” can refer to the complex network of macromolecules filling the extracellular space in a tissue, such as in a nipple, areola or the skin. The extracellular matrix is composed of glycosaminoglycans (GAGs), often covalently linked to protein forming the proteoglycans, and fibrous proteins, including collagen, elastin, fibronectin, and laminin. As the skin is subject to frequent stretching and bending, it has a relatively high content of elastin and elastic fibers. The collagen, for example, can comprise a Type I collagen, a Type III collagen, a Type IV collagen, a Type VI collagen, or a combination thereof. “Extracellular matrix fibrous protein” and “cell adhesion molecule” can refer to a fibrous protein of the extracellular matrix, such as fibronectin, laminin, elastin or a collagen, including those described herein.

The graft can comprise a tissue that is cell free or is substantially free of cells. “Substantially free of cells” can describe a tissue or graft that has had at least 50%, 60%, 70%, 75%, 80%, 85%, 90%, 95%, 96%, 97%, 98%, 99% or more of the cells present in the tissue or structure removed. The percentage reduction in the number of cells can be determined by, for example, counting by visual inspection the number of cells visible in samples pre- and post-decellularization, along with DAPI staining to visualize nuclei. In some embodiments, the graft itself can be free of tissue or substantially free of tissue. For example, “substantially free of tissue” can describe a graft that has had at least 50%, 60%, 70%, 75%, 80%, 85%, 90%, 95%, 96%, 97%, 98%, 99% or more of the tissue or structure removed. In some embodiments, the graft itself can be free of cells or substantially free of cells (e.g., where the graft consists essentially of the extracellular matrix with or without the presence of polymer). For example, “a graft substantially free of cells” can describe a graft where at least 50%, 60%, 70%, 75%, 80%, 85%, 90%, 95%, 96%, 97%, 98%, 99% or more of the cells are absent from the graft structure (e.g., where the graft consists essentially of the extracellular matrix with or without the presence of polymer). In some embodiments, the graft can comprise essentially the extracellular matrix after a tissue has been decellularized. In some embodiments, the graft can comprise essentially the extracellular matrix after a tissue has been decellularized in addition to the presence of polymer.

The graft can comprise a tissue that is free of water (i.e., an anhydrous graft) or one that is substantially free of water. “Substantially free of water” can describe a tissue that has had at least 50%, 60%, 70%, 75%, 80%, 85%, 90%, 95%, 96%, 97%, 98%, 99% or more of the water present in the tissue or structure removed.

The graft can also comprise a tissue that is free of skin, fat, lipids, and/or fibrous tissue. For example, “substantially free of skin, fat, lipids or fibrous tissue” can describe a tissue that has had at least about 50%, about 60%, about 70%, about 75%, about 80%, about 85%, about 90%, about 95%, about 96%, about 97%, about 98%, about 99% or more of the skin, fat, lipids, or fibrous tissue present in the tissue or structure removed.

Plastinization utilizes polymers which permeate into tissues and/or are forcefully impregnated into the tissues to make them stable and free from deterioration. The skilled artisan will recognize that preferred embodiments will utilize a polymer with the following properties to obtain the best results: easy and non-toxic to handle; a low viscosity in the uncured state; a base and a catalyst/resin activator mixture that has a long working time or is in a liquid phase for a long time allowing for impregnation or permeation into the tissues; curing that is not be inhibited by the presence of tissue; appropriate mechanical properties after curing, such as appropriate firmness to stimulate a natural state and/or ability to be repopulated by cells; affordability.

In embodiments, the graft can comprise about 50% polymer or less. For example, the graft can comprise about 0.1% polymer to about 50% polymer. For example, the graft can comprise about 0.1% polymer and about 99.9% extracellular matrix, or the graft can comprise about 1% polymer and about 99% extracellular matrix, or about 50% polymer and about 50% extracellular matrix. In particular embodiments, the graft can comprise about 10% to about 30% polymer. In embodiments, the graft comprises about 10%, about 15%, about 20%, about 25%, or about 30% polymer. In embodiments, the polymer is substantially uniformly distributed in the graft.

In embodiments, the polymer can be provided in a solution that comprises about 50% polymer or less. For example, the solution can comprise about 0.1% polymer to about 50% polymer. If the decellularized tissue is submerged in a bath of 1% polymer, then the amount of polymer within the newly formed “graft” would be less than 1%.

In embodiments, the polymer can be a natural polymer or a synthetic polymer. Natural polymers occur in nature and can be extracted, such as polysaccharides or proteins. Non-limiting examples of polysaccharides comprise chondroitin sulfate, heparin, heparan, alginic acid (i.e., alginate), hyaluronic acid, dermatan, dermatan sulfate, pectin, carboxymethyl cellulose, chitosan, melanin (and its derivatives, such as eumelanin, pheomelanin, and neuromelanin), agar, agarose, gellan, gum, and the like as well as their salt forms (such as sodium salt and potassium salt). Non-limiting examples of proteins comprise collagen, alkaline gelatin, acidic gelatin, gene recombination gelatin, and so on.

Synthetic polymers are man-made molecules formed by the polymerization of a variety of monomers, such as macromolecules comprising polyacrylic acid, polyaspartic acid, polytartaric acid, polyglutamic acid, polyfumaric acid, and so on as well as their salt forms (such as sodium salt and potassium salt). Non-limiting examples of synthetic polymers comprise cyanoacrylate.

In embodiments, the polymer can be a hydrogel. “Hydrogel” can refer to a broad class of polymeric materials with a three-dimensional cross-linked network structure containing a great deal of water, with the state between a liquid and a solid without fluidity.

The polymer can be a colored polymer and/or is capable of being colored, such as by a dye. In embodiments, the polymer comprises melanin, a dye, or a combination thereof. Such polymers dyes are known to those skilled in the art. For example, reference may be made to the following publications: U.S. Pat. Nos. 5,032,670, 4,999,418, 5,106,942, 5,030,708, 5,102,980, 5,043,376, 5,104,913, 5,281,659, 5,194,463, 4,804,719, International Publication No. WO-92/07913

In embodiments, the polymer can be a biodegradable polymer, which can refer to a polymeric material that degrades under aerobic and/or anaerobic conditions in the presence of bacteria, fungi, algae, or other microorganisms. Non-limiting examples of biodegradable polymers comprise chitosan, collagen, alginate, cyanoacrylate, dermabond. In other embodiments, the polymer can be a non-biodegradable polymer, for example, rubber, silicon or UHMWPE.

In still other embodiments, the polymer can be a polymer blend. The concentrations of the various components within the polymer blend will depend on a number of factors, including the desired physical and mechanical properties of the final blend, the performance criteria of articles to be manufactured from a particular blend, the processing equipment used to manufacture and convert the blend into the desired article of manufacture, and the particular components within the blend.

In embodiments, the graft can be seeded with viable cells so as to repopulate the polymer-permeated graft with the viable cells. The term “viable cell” can refer to a cell that is alive and capable of growth, proliferation, migration, and/or differentiation. The decellularized tissues and/or graft can act as structural scaffolds by which viable cells can migrate and readily repopulate. In some embodiments, cells from the native tissue (e.g., the host subject) can also migrate into the structural scaffolds created through the decellularization process and readily repopulate the polymer-permeated graft.

For example, the decellularized tissues and/or the polymer-permeated graft can be seeded and incubated with exogenous cells under conditions conducive to repopulating the decellularized tissue and/or graft with the exogenous cells or cells derived from the exogenous cells. In some embodiments, the exogenous cells can be autologous, homologous (e.g., allogenic), or heterologous. For example, “autologous” refers to biological material (e.g., exogenous cells) that will be introduced into the same individual from whom the material was collected or derived. For example, “homologous” can refer to biological material (e.g., exogenous cells) collected or derived from a compatible donor that will be introduced into a different individual from which the material was collected or derived. For example, “heterologous” can refer to biological material (e.g., exogenous cells) collected or derived from a compatible donor of a different species that will be introduced into an individual. Non-limiting examples of exogenous cells that can be seeded onto (and thus useful for repopulating the decellularized tissue and/or polymer-permeated graft) include keratinocytes, melanocytes, nerve cells, stromal cells, fibroblasts, endothelial cells, progenitor cells, stem cells, organ-specific cells, tissue-specific cells, or a combination thereof. In some embodiments, keratinocytes readily migrate and repopulate decellularized dermis and/or decellularized epidermis. In some embodiments, melanocytes readily migrate and repopulate the decellularized tissue and/or polymer-permeated graft. In some embodiments, nerve cells readily migrate and repopulate the decellularized tissue and/or polymer-permeated graft. In further embodiments, the nerve cells can be neurospheres or neuronal cells. For example, “exogenous” relates to cells that have been introduced (e.g., seeded) to recellularize or repopulate a decellularized tissue such that the cells that did not originate in the decellularized tissue. Without being bound by theory, if a nipple from a subject is decellularized and repopulated with keratinocytes, melanocytes, and/or nerve cells originating from a skin punch taken from the same subject, the keratinocytes, melanocytes, and/or nerve cells are still exogenous to the decellularized nipple because they did not originate from the nipple.

Conditions conducive to repopulate the graft are dependent upon the cells used, and can include temperature, the presence or absence of growth factors, the presence or absence of differentiation factors or migration factors, the polymer used to permeate the tissue, or the air content. In embodiments, the polymer-permeated graft is introduced or implanted onto a subject, and the subject's own cells migrate into the graft. In other embodiments, viable cells are introduced into the tissue or graft prior to implanting the graft onto the subject.

One of skill in the art can seed exogenous cells onto the decellularized tissue and/or graft by placing the structures into culture medium containing dissociated, or dissociated and expanded, cells and allowing the cells to migrate into the decellularized tissue or graft and repopulate the structures. In some embodiments, cells can be injected into one or more places in the decellularized tissue or graft, such as into the interior, in order to accelerate repopulation of the structures.

The viable cells can be cultured prior to reseeding of the tissue or graft. The culture medium used to grow and expand cells of interest can be serum-free and would not require the use of feeder cells. Suitable media specific for keratinocytes are known in the art and include (but are not limited to): Keratinocyte Growth Medium 2 (PromoCell GmbH, Heidelberg, Germany); Stemline™ keratinocyte basal medium (Sigma-Aldrich Corp., St. Louis, Mo.); defined, BPE-free medium supplement (K 3136) (Sigma-Aldrich Corp.), and ATCC's Dermal Cell Basal Medium (PCS-200-030) supplemented with Keratinocyte Growth Kit (PCS-200-040). Suitable media specific for melanocytes are known in the art and include (but are not limited to) growth medium comprising insulin, ascorbic acid, glutamine, epinephrine, and calcium chloride. See, for example, ATCC Melanocyte Growth Kit (ATCC-PCS-200-041). Suitable media specific for nerve cells are known in the art and include (but are not limited to) growth medium comprising DMEM, 10% FBS, supplemented with NGF and L-glutamine. The polymer can be admixed with at least one antibiotic. “Antibiotic” can refer to a substance that controls the growth of bacteria, fungi, or similar microorganisms, wherein the substance can be a natural substance produced by bacteria or fungi, or a chemically/biochemically synthesized substance (which may be an analog of a natural substance), or a chemically modified form of a natural substance. One of skill will recognize that the polymer can be admixed with a wide variety of antibiotics, such as penicillins, cephalosporins, macrolides, fluoroquinolones, sulfonamides, tetracyclines, aminoglycosides, and the like.

Embodiments can comprise ABGs impregnated with biodegradable polymers, such as those described herein, that can provide enhanced mechanical properties. Polymer impregnation of an ABG can itself enhance the mechanical properties of the graft, allowing it to be more durable than a non-impregnated graft and thus prevent graft failure due to mechanical forces.

In further embodiments, the ABG and/or the polymer-impregnated ABG can further comprise therapeutics and/or drugs, such as for the sustained or controlled release of such therapeutics and/or drugs. Such agents can be used to prevent and/or treat progression and/or symptoms of disease (such as those diseases and symptoms described herein), and can also be used to prevent, treat, and or alleviate unwanted side effects of graft implantation. Non-limiting examples of unwanted side effects of ABG implantation or grafting, for example, comprise pain, infection, inflammation, or scarring. Such unwanted side effects can be prevented, treated, or relieved through sustained, controlled, local release of drugs and/or therapeutic agents from the polymer or the ABG. For example, the addition of at least one anti-biotic, at least one anti-inflammatory, and/or at least one analgesic and/or anesthetic could prevent infection, reduce local inflammation and decrease pain at the surgical and/or implantation site, thus, for example, providing symptomatic relief.

ABG and/or polymers can mixed with therapeutic and/or prophylactic agents allowing for sustained release of the therapeutic and or prophylactic agent. Non-limiting examples of such agents comprise antibiotics, pain relievers, anti-inflammatories, or any combination thereof.

“Antibiotic” can refer to an agent that controls the growth of bacteria, fungi, or similar microorganisms, wherein the substance can be a natural substance produced by bacteria or fungi, or a chemically/biochemically synthesized substance (which may be an analog of a natural substance), or a chemically modified form of a natural substance. One of skill will recognize that the scaffold can be coated with a wide variety of antibiotics, such as penicillins, cephalosporins, macrolides, fluoroquinolones, sulfonamides, tetracyclines, aminoglycosides, and the like.

“Pain reliever” can refer to an agent that can provide relief from pain. An analgesic is any member of a group of drugs used to achieve analgesia, i.e., relief from pain. For example, the analgesic can be a pyrazolone derivative, such as (ampyrone, dipyrone, antipyrine, aminopyrine, and propyphenazone), aspirin, paracetamol, a non-steroidal anti-inflammatory (such as Ibuprofen, diclofenac sodium, or naproxen sodium), an opioid (such as codeine phosphate, tramadol hydrochloride, morphine sulphate, oxycodone), or any combination thereof. An anesthetic refers to any member of a group of drugs used to induce anesthesia—in other words, to result in a temporary loss of sensation or awareness of pain. Non-limiting examples of anesthetics comprise benzocaine, chloroprocaine, cocaine, cyclomethycaine, dimethocaine, larocaine, piperocaine, propoxycaine, procaine, novocaine, proparacaine, tetracaine, amethocaine, articaine, bupivacaine, cinchocaine, dibucaine, etidocaine, levobupivacaine, lidocaine, lignocaine, mepivacaine, prilocaine, ropivacaine, trimecaine.

An “anti-inflammatory” refers to a substance that treats or reduces the severity of inflammation and/or swelling. Non-limiting examples of anti-inflammatories comprise steroidal anti-inflammatories (such as corticosteroids) and non-steroidal anti-inflammatories (such as aspirin, celecoxib, diclofenac, diflunisal, ibuprofen, indomethacin, ketoprofen, ketorolac, nabumetone, naproxen, oxaprozin, piroxicam, salsalate, sulindac, tolmetin).

Sustained-release grafts have a common goal of improving treatment and/or symptomatic relief over that achieved by their non-controlled counterparts. The use of an optimally designed sustained-release preparation in medical treatment can be characterized by a minimum of drug substance being employed to cure, control, and/or provide relief of the condition in a minimum amount of time. For example, the sustained-release grafts can release an amount of a drug over the course of 1 day, 1 week, 2 weeks, 3 weeks, 4 weeks, 2 months, 3 months, 4 months, 5 months, 6 months, 7 months, 8 months, 9 months, 10 months, 11 months, 12 months, or longer. Advantages of sustained-release formulations include extended activity of the drug, reduced dosage frequency, and increased patient compliance. In addition, sustained-release formulations can be used to affect the time of onset of action or other characteristics, such as blood levels of the drug, and can thus affect the occurrence of side (e.g., adverse) effects.

Most sustained-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released at a rate that will replace the amount of drug being metabolized and excreted from the body. Sustained-release of an active ingredient can be stimulated by various conditions including, but not limited to, pH, temperature, enzymes, water, or other physiological conditions or compounds.

The polymer can be capable of cross-linking. A cross-link, or cross-link, refers to a bond, such as covalent bods or ionic bonds, that links one polymer chain to another. Cross-linking can promote a difference in the polymers' physical properties. For example, a liquid polymer or resin can be turned into a semi-solid (i.e., gel) or solid by cross-linking the chains together. The resulting modification of mechanical properties depends strongly on the cross-link density. Low cross-link densities decrease the viscosities of polymer melts. Intermediate cross-link densities transform gummy polymers into materials that have high elastomeric properties and potentially high strengths. Very high cross-link densities can cause materials to become very rigid or glassy, such as phenol-formaldehyde materials. The skilled artisan will recognize that the extent of cross-linking and the specificities of cross-linking will vary based on the polymers utilized, the cross-linking methods utilized, and the desired outcome.

Plastinized anhydrous grafts described herein can be stored more easily and for longer periods of time than non-plastinized grafts. The storage conditions can be dependent on the properties of the graft itself, such as the polymer and/or the tissue utilized. As examples, the plastinized grafts can be stored either hydrated, dehydrated, or partially hydrated. For example, a hydrated graft can be stored in glycerol, saline, water, or organ preservation solution (such as perfadex). The grafts can be stored at any temperature between about −80° C. and about 4° C. The grafts can be lyophilized.

### Methods of Decellularization

Aspects of the invention are directed towards polymer-permeated grafts that comprise a tissue free or substantially free of cells (i.e., decellularized). “Decellularization” of a biological tissue or structure can refer to removing most or all of the cells of the tissue or structure. See, for example, U.S. patent application Ser. No. 15/523,306, which is incorporated by reference herein in its entirety.

A “decellularized” biological tissue or structure (such as the dermis or epidermis herein), for example, can refer to removing most or all of the cells of the tissue or structure while the extracellular matrix (ECM) is substantially preserved in addition to cell adhesion molecules. The extracellular matrix is a complex network of macromolecules filling the extracellular space in a tissue (such as the dermis and/or epidermis that can comprise a nipple and/or areola). The extracellular matrix has three main components: (1) viscous proteoglycans (e.g., glycosaminoglycans (GAGs) covalently linked to proteins), such as hyaluronan, heparan sulfate, keratan sulfate, chondroitin sulfate, and dermatan sulfate; (2) insoluble collagen fibers (proteins that provide strength) and elastin (proteins that provide resilience); and (3) soluble, fibrous ECM proteins (including fibronectin, and laminin) that bind proteoglycans and collagen fibers to receptors on the cell surface. An “extracellular matrix fibrous protein” and “matrix molecule” each can refer to a fibrous protein of the extracellular matrix, such as fibronectin, laminin, elastin or collagen. In some embodiments, collagen can comprise a Type I collagen, a Type III collagen, a Type IV collagen, a Type VI collagen, or a combination thereof.

For example, a tissue can be removed from a patient (e.g., self or non-self), from a cadaver, or from a non-human primate. Such removed tissues/structures can be referred to as a “donor tissue”, and can be decellularized while retaining their natural gross structures, microarchitecture, and matrix molecules, including collagen, fibronectin, elastin and glycosaminoglycans.

Aspects of the invention are further directed towards preparing and/or using polymer-permeated grafts that comprise a tissue that has been substantially decellularized. “Decellularizing substantially all” cells of a described tissue or structure means that at least 50%, 60%, 70%, 75%, 80%, 85%, 90%, 95%, 96%, 97%, 98%, 99% or more of the cells present in the tissue or structure have been removed. The percentage reduction in the number of cells can be determined by, for example, counting by visual inspection the number of cells visible in samples pre- and post-decellularization, along with DAPI staining to visualize nuclei.

Others have reported decellularization of organs, as exemplified by U.S. Pat. No. 8,470,520, the entirety of which is incorporated herein by reference. The organs whose decellularization is reported in this patent include lung, but not skin. Our protocols for decellularizing the lung and organs comprised the following steps. We first contacted the lung with a dilute solution of a detergent or surfactant capable of permeating eukaryotic cell membranes and solubilizing membrane proteins and incubated it at 4° C. The detergent or surfactant was changed each day. After 4 days, the samples were washed with water for several hours and then contacted and incubated with a 2% solution of a bile salt, sodium deoxycholate (“SDC”), for 4 days at 4° C., with the bile salt solution changed each day. The samples were then subjected to a second wash with water, incubated with DNase I for 2 hours, again at 4° C., washed again with water and then stored. The solutions were introduced into the lung by perfusion.

We found that a protocol we had previously used successfully to decellularize organs such as the lung did not work to decellularized nipple epidermis in studies using Rhesus macaque nipples. Specifically, the protocol that had successfully decellularized the lung succeeded in decellularizing approximately 95% of the dermis cells, but only 5% of the epidermal cells.

AlloDerm® and Glyaderm®, two commercially available acellular dermal matrices, are used as tissue extenders or dermal replacement for wounds and burns. Neither contains acellular epidermis. It is believed that these materials are made by separating the epidermis from the dermis prior to decellularizing the dermis. Without wishing to be bound by theory, it is believed that this is in part because the epidermis is denser than the dermis and much harder to decellularize.

To solve this problem, we performed studies to find protocols that would succeed in decellularizing nipple epidermis. After consideration experimentation, we succeeded in developing modifications that successfully decellularized nipple epidermis, along with the accompanying dermis. Given our results with decellularizing nipple epidermis and dermis, we expect the protocols to work equally well in decellularizing skin epidermis and dermis. In some embodiments, the epidermis of the donor tissue is decellularized along with the dermis.

To decellularize nipples, including the epidermis, we modified the protocol in multiple ways. First, the temperatures of the incubations and washes were raised—rather than conducting them at 4° C., they were conducted at room temperature. Second, rather than changing the solutions each day during the multiple-day incubations, the samples were left in the same solution throughout the incubation. Without wishing to be bound by theory, it was thought this would augment digestion of the cells by not removing any endogenous proteases present in the cells. Third, the times of the incubations were doubled. Fourth, the concentration of the bile salt was doubled, with the concentration of the bile salt raised from 2% to 4%. Fourth, for the lung, the organ was perfused. As nipples do not have vessels allowing ready perfusion, the samples were initially agitated on an orbital shaker set to a rotation speed of 85-125 rpm, which we thought would subject the nipple to solutions in a manner simulating perfusion. We found, however, that the nipple did not decellularize at low speed agitation, but did when the shaker speed was increased to 325 rpm. While none of these changes by themselves or any two or three together were sufficient to decellularize the nipple samples, the combination of all four succeeded.

As the original decellularization protocol worked on the lung, which has airways permitting essentially all the tissue of the organ to be contacted with the detergents and other reagents, but did not work when used on the nipple, the nipple appears to be particularly resistant to decellularization of the epidermis. We believe that the protocol developed for decellularizing the nipple can therefore be used to decellularize other body parts for which an acellular matrix might be useful.

Based on the studies undertaken on the nipple, a sample to decellularized is contacted with a first detergent or surfactant solution for 48 hours to about 144 hours, more preferably about 72 to about 120 hours, still more preferably about 80 to about 110 hours, even more preferably for about 96 hours, where “about” means±2 hours, which detergent or surfactant can permeate eukaryotic cell membranes and solubilize membrane proteins. Suitable detergents include 4-(1,1,3,3-Tetramethylbutyl)phenyl-polyethylene glycol (Triton™ X-100), octylphenoxypolyethoxy-ethanol (IGEPAL® CA-630), CHAPS (3-[(3-Cholamidopropyl)dimethylammonio]-1-propanesulfonate), sodium dodecyl sulfate and polyethylene glycol, with the first three being more preferred. The sample is then washed with water, preferably for several hours, and then contacted with an appropriate soluble bile salt as a second detergent for a time period similar to those described above for use with the first detergent, above. Sigma-Aldrich Corp. (St. Louis, Mo.), for example, sells at least the following sodium salts of bile acids: sodium cholate, sodium deoxycholate, sodium glycocholate, sodium taurocholate, and sodium taurodeoxycholate. While all of these salts have sodium as the cation, other cations can be used to form a salt of a bile acid for use in the inventive methods so long as the resulting bile salt is soluble and decellularizes epidermis. Any particular bile salt can be readily tested to see whether it is suitable for its use in decellularizing epidermis by using it in place of SDC in the decellularization protocol set forth in the Examples and subjecting the resulting sample to histological or immunological examination, or both, to determine whether substantially all epidermal cells of the nipple have been removed. If it has, the bile salt is suitable for use in the protocol.

The sample is then again washed with water for a few hours, preferably about two, and then washed with a saline solution for a few hours, preferably about two, where “about” means plus or minus 15 minutes. The samples are then incubated overnight with 5×streptomycin-penicillin-amphotericin B (sold by a number of suppliers at 100×, including Sigma Aldrich, Lonza Walkerville, Inc. (Walkerville, Md.), the American Type Culture Collection (“ATCC”, Manassas, Va.), and EMD Millipore (Billerica, Mass.)), water washed, treated with deoxyribonuclease I (“DNase I”) for several hours, preferably about two hours, and then stored in a phosphate buffered saline solution containing 5×streptomycin-penicillin-amphotericin B at 4° C. until use. Using an orbital shaker as an exemplar, the rotations per minute, or rpm, is set to between about 250 and about 400, more preferably about 275 to about 375, still more preferably about 300 to about 350 and most preferably about 325, with “about” in this case meaning 5 rpm on either side of the stated number. Orbital shakers are preferred for their smooth continuous motion and uniform mixing. A number of other shakers are known in the art, including rocking, rolling, reciprocal, overhead, vibrating platform, and rotating shakers. Additionally, other devices, such as rotators, that allow uniform mixing contents of containers over time are known. In general, any shaker, rotator or similar device that provides mechanical agitation of a sample can be used so long as it provides uniform mixing without being so violent that the mixing action disrupts the physical integrity of the sample, such as a nipple or NAC, being decellularized. Depending on the type of shaker or rotator, the speed of the agitation of containers holding the sample may be stated in units other than rpms. It is anticipated that the person of skill will be readily able to determine the appropriate speed setting for the particular shaker or rotator used by reference to the rpm setting for an orbital shaker, as set forth above.

The use of decellularized tissues provide a better scaffold for reseeding and formation of a graft for in vivo use. As persons of skill will understand, a tissue can be decellularized and then repopulated or recellularized. A recellularized tissue or structure is not expected to have the combination of cell types that may have been present in the structure before it underwent decellularization. For example, the decellularized tissue or graft can be repopulated using exogenous cells. “Exogenous” in relation to cells introduced to recellularize a decellularized tissue means cells that did not originate in the decellularized tissue. By way of example, if a nipple from a patient is decellularized and repopulated with keratinocytes originating from a skin punch taken from the same patient, as used herein, the keratinocytes are still exogenous to the decellularized nipple because they did not originate from the nipple.

Methods of Plastination

Aspects of the invention are directed towards methods of plastinizing tissues so as to provide a plastinized (i.e., polymer-permeated) graft for in vivo use. Steps of the plastination process can comprise fixation, dehydration, forced impregnation in a vacuum, and hardening. See, for example, Prasad, Ganesh, et al. “Preservation of tissue by plastination: A Review.” Int. J. Adv. Health Sci 1.11 (2015): 27-31; and U.S. Pat. No. 4,278,701, each of which are incorporated by reference herein in their entireties.

Fixation

Fixation of a decellularized tissue can refer to a process utilized to kill all microorganisms so as to prevent decomposition of the tissues. In embodiments, tissue fixation may not be required. The fixation process generally comprises submerging, immersing, or perfusing the tissue with a fixative for a period of time sufficient to fix the tissue. The skilled artisan will recognize that the period of time sufficient to fix the tissue can depend upon the rate of penetration of the fixative, the type of tissue to be fixed, the size of the tissue to be fixed, the concentration of the fixative and additives to be used, the type of the fixative used, temperature, tonicity, and the like. Non-limiting examples of fixatives that can be used in the methods described herein comprise aldehydes (such as formaldehyde or glutaraldehyde), genipin, or combinations of two or more fixatives. Generally, the fixative process can take about 3 to 4 hours, but can be shorter or longer as needed.

Dehydration

“Dehydration” refers to a process by which all or substantially all of the water is removed from the decellularized tissue to provide a tissue that is free of or substantially free of water. “Substantially free of water” can describe a tissue that has had at least 50%, 60%, 70%, 75%, 80%, 85%, 90%, 95%, 96%, 97%, 98%, 99% or more of the water present in the tissue or structure removed.

In embodiments herein, all or substantially all of the water within the decellularized tissue is removed and/or replaced with an organic solvent by submerging, immersing, or perfusing the tissue with the solvent for a period of time and at a temperature sufficient to remove and/or replace all or substantially all of the water within the tissue.

In embodiments, all or substantially all of the lipids and/or fat within the tissue can be removed and/or replaced with an organic solvent by submerging, immersing, or perfusing the tissue with the solvent for a period of time and at a temperature sufficient to remove and/or replace all or substantially all of the fat and lipid within the tissue.

Non-limiting examples of organic solvents comprise acetone, xylene, dichloromethane (i.e., DCM or methylene chloride), and/or alcohols such as ethanol or methanol. The organic solvent will draw out all or substantially all of the water from the tissue, and replace it inside of the tissue.

The skilled artisan will recognize that the period of time and temperature sufficient to replace and/or remove water from the tissue can depend upon the rate of penetration of the solvent, the type of tissue, the size of the tissue, the concentration of the solvent and any additives to be used, the type of solvent used, tonicity, and the like. For example, the decellularlized tissue can be incubated in acetone at about −15° C. to 25° C. for a period of time as short as about 5 minutes to as long as about 10 days.

In embodiments, water in a tissue can be removed and/or replaced by sequential immersion of the tissue in repeated changes of organic solvents until all or substantially all of the water within the tissue is removed or replaced. The same organic solvent can be used in each bath, or different solvents can be used in the baths, such as beginning with the least expensive solvent, such as aqueous ethanol, and progressing gradually to anhydrous ethanol and/or acetone which may then be followed by other organic solvents to suit processing conditions, that is, solvents which are either compatible with the polymer precursor and with the solid polymer to be obtained therefrom by polymerization, or which are at least compatible with the precursor and volatilize prior or during curing.

Forced Permeation

Replacement of the solvent by polymer is a central step in plastination, and generally comprises submerging or immersing the decellularized tissue in a bath of liquid polymer for a period of time under vacuum conditions. As the solvent leaves the tissue, such as vaporizing, liquid polymer is drawn into the tissue so that the polymer can permeate and/or impregnate the tissue. The polymer can be substantially uniformly distributed within the tissue.

In embodiments, the polymer can comprise those described herein, non-limiting examples of which comprise acrylic resins, epoxy resins, polyester resins, polyurethanes, and silicone resins varying widely in their chemical properties and their processing characteristics, particularly in the conditions under which they are formed by polymerization of monomers or intermediates.

The solvent-bearing tissue can be permeated and/or impregnated with a polymer of precursor thereof by submerging, immersing or perfusing the tissue with a fluid composition comprising the polymer or precursor thereof, and any other additives such as catalysts or hardeners, chemical cross-linking agents (such as glutaraldehyde, carbodiimide (1-ethyl-3-(3-dimethyl aminopropyl)-carbodiimide), epoxy compounds, six methylene diisocyanate, glycerin, alginate, genipin, ordihydroguaiaretic acid, proanthocyanidin, tannic acid, and epigallocatechin gallate), accelerators, plasticizers, and like conventional ingredients. Impregnation of the immersed tissue can be aided by evaporating or otherwise releasing the organic solvent from the tissue, such as by vacuum, as the solvent can be more volatile than any component of the polymer solution. For example, exposing the tissue immersed in the polymer to a vacuum can cause impregnation or permeation in a very short time if the solvent is volatile in the vacuum, and the precursor composition is not overly viscous. Compositions up to about 5000 cps have been used without difficulty, and even more viscous compositions may be employed for impregnation by alternating application of negative and positive pressure. Much lower viscosity is necessary for successful impregnation by perfusion.

For example, the tissue, immersed and/or submerged within a volatile solvent can be removed from the solvent and placed into the polymer solution. The solvent can have a high vapor pressure and a low boiling point (for example, acetone: +56° C., methylene chloride: +40° C.), while the polymer solution has a low vapor pressure and a high boiling point. Thus, on application of vacuum, the solvent is continuously extracted out of the specimen as gaseous bubbles. As the solvent leaves the tissue, a void is produced in the tissue and the synthetic resin is drawn into the specimen.

The skilled artisan will recognized that the rate of extraction of solvent from the tissue can vary, and can depend on properties of tissue to be impregnated and/or the solvent to be extracted. The rate of extraction of the solvent can be visualized when bubbles gently rise to the surface and burst. When no more bubbles appear, the process is complete.

To monitor the changes in vacuum, initially a vacuum gauge or an Hg column is used and later a manometer is used. The impregnation or permeation is said to be complete when the absolute pressure has stabilized to around 2-10 mm Hg for a few days. Furthermore, on visualization of the bubbles, the small bubbles measuring about 1-1.5 cm are now large 4-5 cm, which is water vapor. In embodiments, the tissue can be left in the impregnation bath at atmospheric pressure for a period of time, such as 24 hours, to allow the equilibration of pressure of the polymer in the specimen and in the impregnation bath. After this step, the tissue can be removed.

Cross-Linking

After immersion in a polymer solution, the tissue can be removed from the polymer and excess polymer can be drained off or removed by wiping and the like before polymerization conditions (i.e., cross-linking conditions or curing conditions) are established. Cross-links can be formed by chemical reactions that are initiated by gas, light, heat, pressure, change in pH, radiation, cross-linking agents, and the like. Crosslinks are useful for preventing degeneration of the structural integrity of the scaffold that remains after decellularization, enhancing mechanical strength and reducing calcification of the matrix. For example, mixing of an unpolymerized or partially polymerized monomer with cross-linking agents can result in a chemical reaction that forms cross-links (i.e., polymerizes). The skilled artisan will recognize that the extent of cross-linking and the specificities of cross-linking will vary based on the polymer and precursors utilized, the cross-linking methods utilized, the cross-linking agents utilized, and the desired outcome.

An ideal biomaterial crosslinking agent demonstrates little to no cytotoxicity and is low cost. There are many crosslinking agents for fixing ECM-derived scaffolds, which may be classified as (i) chemical crosslinking agents and (ii) natural crosslinking agents. The chemical crosslinking agents comprise glutaraldehyde (GA), carbodiimide (1-ethyl-3-(3-dimethyl aminopropyl)-carbodiimide (EDC)), epoxy compounds, six methylene diisocyanate, glycerin and alginate, for example; and the natural crosslinking agents include genipin (GP), nordihydroguaiaretic acid (NDGA), tannic acid and procyanidins (PC), for example. See Ma, Bing, et al. “Crosslinking strategies for preparation of extracellular matrix-derived cardiovascular scaffolds.” Regenerative biomaterials 1.1 (2014): 81-89, which is incorporated by reference here in its entirety.

Non-limiting examples of a cross-linking agent include glutaraldehyde, carbodiimide (1-ethyl-3-(3-dimethyl aminopropyl)-carbodiimide), epoxy compounds, six methylene diisocyanate, glycerin, alginate, collagen, genipin, ordihydroguaiaretic acid, proanthocyanidin, tannic acid, and epigallocatechin gallate.

In embodiments, the surface and mechanical properties of the finished product (i.e., the polymer permeated graft) is to duplicate or mimic those of the fresh tissue. For example, silicone rubber precursor compositions of low viscosity are exemplary polymers for producing resilient objects duplicating the surface configuration of fresh tissue, and the resiliency of the cured silicone rubber simulates the softness of the fresh tissue to some reduced degree.

Cross-linking can depend upon the polymer utilized. For example, gas curing can be used for plastination using silicone resin. In this technique, the decisive cross-linking curing agent is applied in a gaseous form to the tissue. The silicone-impregnated tissue is kept in a closed chamber and are exposed to a gaseous hardener which, on evaporation from a stock solution, is continuously circulating in the atmosphere of the chamber. A small membrane pump helps in the evaporation and circulation of the gas, leading to faster curing.

As another example, the curing of an epoxy-impregnated tissue or polymerizing emulsion-impregnated tissue can use the tissue amines present within the tissue itself for curing. These amines are effective accelerators and together with anhydrides, they are sufficient to fully cure the tissues.

As yet another examples, polymerization can be induced by exposure to a radiation source, such as electron beam exposure, gamma-radiation, or UV light. For example, electron beam processing can be used to cross-link the C type of cross-linked polyethylene. Other types of cross-linked polyethylene are made by addition of peroxide during extruding (type A) or by addition of a cross-linking agent (e.g. vinylsilane) and a catalyst during extruding and then performing a post-extrusion curing.

Polymerization conditions are chosen to suit the specific polymer and/or precursor composition employed within limits set by the need to avoid decomposition of the tissue, and can include elevated temperatures, irradiation with ultraviolet light, the presence of initiators or catalysts, and other factors known in themselves. If atmospheric oxygen unduly inhibits the curing of polyester resin, the impregnated object can be immersed in anhydrous glycerol or held in a nitrogen atmosphere during curing. As an example, the polymerization of silicone rubber is not inhibited by oxygen in the atmosphere, but can be retarded as needed by maintaining a low temperature (below 32° F.) during impregnation. Their index of refraction (n=1.405) enhances the natural appearance of the impregnated tissue surface, and the resiliency of the cured silicone rubber simulates the softness of the fresh tissue to some reduced degree.

In embodiments, for example when polyester or epoxy resins are employed as the polymer, the temperature of the polymer fluid composition can be monitored to avoid polymerization and a sharp increase in viscosity before excess resin precursor is removed.

Methods of Treatment

Aspects of the invention are directed towards methods of using a polymer-permeated graft (e.g., an acellular biologic graft (ABG) described herein) to treat a subject in need thereof. The ABGs according to the invention can be alternatives to synthetic mesh implants for use, e.g., in pelvic organ prolapse (POP) and abdominal wall repair, hernia repair. The ABGs according to the invention can also serve as alternatives to slings for use, e.g., in breast reconstruction. Further, the ABGs according to the invention can be alternatives to supplemental supports and/or coverings for tissue (for example, replacement of synthetic of biologically derived grafts) for use with gums, tendons, breast, burns, and the like. For example, the subject may be in need of nipple repair or replacement, breast reconstruction, hernia repair or replacement, blood vessel repair or replacement, muscle repair or replacement, repair or replacement of whole organs, tendon repair or replacement, cleft lip repair or replacement, or palate repair or replacement. In some embodiments, an ABG can be used to treat pelvic organ prolapse (POP) in a subject, for example, for use in a subject undergoing a POP surgery.

In embodiments, the method can comprise obtaining a plastinized graft such as a polymer-permeated graft as described herein and securing the graft to a prepared site on the patient. In some embodiments, the method further comprises allowing time for cells from the patient to integrate into the body part.

The term “treating” can refer to partially or completely alleviating, ameliorating, improving, relieving, delaying onset of, inhibiting progression of, reducing severity of, and/or reducing incidence of one or more symptoms, features, or clinical manifestations of a particular disease, disorder, and/or condition. Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition.

The term “subject” or “patient” can refer to any organism to which aspects of the invention can be administered, e.g., for experimental, diagnostic, prophylactic, and/or therapeutic purposes. Typical subjects to which compositions of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted above or another organism that is alive. The term “living subject” refers to the entire subject or organism and not just a part excised (e.g., a liver or other organ) from the living subject.

As used herein, “changed as compared to a control” sample or subject is understood as having a level of an analyte or diagnostic or therapeutic indicator (e.g., marker) to be detected at a level that is statistically different than a sample from a normal, untreated, or abnormal state control sample. The diagnostic or therapeutic indicator can be assessment of the growth of the tissue grafted or observation for lack of graft rejection. Determination of statistical significance is within the ability of those skilled in the art, e.g., the number of standard deviations from the mean that constitute a positive or negative result.

In embodiments, the polymer-permeated graft can be implanting onto a prepared site on or within a subject in need thereof; thereby grafting to a subject the polymer-permeated graft.

In embodiments, the graft has been repopulated with viable cells as described herein.

In other embodiments, the decellularized tissue has been not been repopulated or has only been partially repopulated by cells (such as keratinocytes, melanocytes, nerve cells, or a combination thereof either that were seeded or that migrated from the native tissue) before grafting onto a subject in need thereof.

Cells from the prepared bed (such as keratinocytes, skin stem cells, melanocytes, nerve cells, and fibroblasts) can migrate into the polymer-permeated graft and repopulate it. In some embodiments, the migration of cells into the graft is facilitated by (a) placing the graft on the subject on the prepared bed and (b) coating the graft and the junction where the graft adjoins the subject's skin with a biocompatible substance. For example, the biocompatible substance (or occlusive coating) can be a tissue sealant, a tissue adhesive, tissue glue, or a surgical glue. For example, “biocompatible” refers to a material which is not toxic, not injurious or not inhibitory to mammalian cells, tissues, or organs with which it comes in contact. Furthermore, when the material is in use with respect to a graft does not induce an immunological or inflammatory response sufficient to be deleterious to the subject's health or to engraftment of the graft. Other biocompatible occlusive coatings that provide an air sealing barrier, such as Fibrin glues, can be used. A fibrin sealant, TISSEEL®, is commercially available, as are the sealants BIOGLUE® and DuraSeal®. A non-limiting example of a tissue sealant includes high viscosity 2-octyl cyanoacrylate (for example, sold commercially under the names DERMABOND® (Ethicon unit of Johnson & Johnson) and Sure+Close®II).

Once the polymer-permeated graft is grafted onto the subject, it can be covered with a biocompatible occlusive coating as described herein. The skilled artisan can readily obtain keratinocytes, melanocytes, nerve cells, or a combination thereof, from one or more skin punches (either from the same subject or from a compatible donor) according to methods and teachings known in the art. The keratinocytes, melanocytes, nerve cells, or a combination thereof, can be placed in a culture medium suitable for maintenance and stability, from which the cells can then permeate into the graft. In some embodiments, these cells can also be injected into the graft at one or more locations. Grafts of the invention can be maintained in a cell culture medium suitable for maintenance and expansion of keratinocytes (human or non-human); cell culture medium suitable for maintenance and expansion of melanocytes (human or non-human); cell culture medium suitable for maintenance and expansion of nerve cells (human or non-human). The culture medium used to grow and expand cells of interest can be serum-free and would not require the use of feeder cells.

Kits

The compositions and grafts as described herein can also be provided in a kit. In one embodiment, the kit includes (a) a container that contains a composition or a graft as described herein, and optionally (b) informational material. In another embodiment, the kit comprises vials comprising (a) a fixative, an organic solvent, a polymer, a chemical linker, or any combination thereof, and (b) informational material. The informational material can be descriptive, instructional, marketing or other material that relates to the methods described herein and/or the use of the composition, the graft for therapeutic benefit, or solutions. In an embodiment, the kit also includes a biocompatible sealant for treating a subject in need thereof.

The informational material of the kits is not limited in its form. In one embodiment, the informational material can include information about production of the composition or the graft, components of the composition or the graft, date of expiration, batch or production site information, and so forth. In one embodiment, the informational material relates to methods of administering or affixing the composition or the graft, e.g., in a suitable form, or mode of administration, to treat a subject. The information can be provided in a variety of formats, include printed text, computer readable material, video recording, or audio recording, or information that provides a link or address to substantive material. In addition to a composition or graft as described herein, the composition in the kit can include other ingredients, such as a buffer, a stabilizer, or a preservative. The composition or graft can be provided in a sterile form and prepackaged.

The kit can include one or more containers for the composition or grafts described herein. In some embodiments, the kit contains separate containers, dividers or compartments for the composition or graft and informational material. For example, the composition can be contained in a culture plate, and the informational material can be contained in a plastic sleeve or packet. In other embodiments, the separate elements of the kit are contained within a single, undivided container. For example, the composition or graft is contained in a container or culture plate that has attached thereto the informational material in the form of a label. The containers of the kits can be air tight, waterproof (e.g., impermeable to changes in moisture or evaporation), and/or light-tight.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Plastination Protocol and Methods for Tissue Engineering

Step 1: Obtain a Decellularized Tissue.

For example, the tissue can be decellularized using the protocol as described in U.S. patent application Ser. No. 15/523,306, the entirety of which is incorporated herein by reference. Generally, skin, fat, and fibrous tissues are removed prior to the decellularization process.

Step 2: Optionally, Fix the Tissue.

For example, the tissue can be fixed in an aldehyde, such as formaldehyde or glutaraldehyde.

Step 3: Submerge the Tissue in a Solvent to Ensure the Water within the Tissues is Replaced.

The tissue can be submerged in the solvent in a series of baths to ensure the water within the tissues is replaced. The water content in the baths can be measure before the tissue is submerged and timepoints while the tissue is submerged to determine when this step is complete. When all or substantially all of the water is removed from the bath, the process is complete.

Solvents can comprise organic compounds such as acetone or alcohols (such as ethanol), or solvents that become gaseous under vacuum. For example, the tissue can be submerged in a cold acetone bath (for example approximately −15° C.-25° C.) for a period of time. The skilled artisan will recognize that time and temperature of the solvent baths will vary depending upon the polymer to be used, whether or not cells will be used to repopulate the tissue.

Optionally, solvent baths can be used to dissolve soluble body fat within the tissue. For example, acetone can be warmed to room temperature to dissolve soluble body fat.

Step 4: Forced Vacuum Impregnation.

The sample is submerged in a bath of a polymer or resin and subject to vacuum. The process of forced vacuum impregnation is complete when no bubbles are observed.

The skilled artisan will recognize that a variety of polymers and/or resins can be used. For example, medical grade silicone can be used.

Different types of polymers can be utilized which encourage different types of cell growth. For example, alginate can be used as the polymer to stimulate neurite growth.

In embodiments, polymers can be mixed with other components, such as cells or antibiotics. For example polymer mixed with cells can be used to seed new cells for tissue engineering application throughout biomaterials. At present, this is an unmet challenge for tissue engineering. As another example, polymers can be mixed with therapeutic and/or prophylactic agents such as pain relievers, anti-inflammatories, antibiotics, antifungals, or antimicrobials, allowing for sustained release of the therapeutic and or prophylactic agent.

In embodiments, the polymer can be biodegradable polymer (such as chitosan, collagen, alginate, cyanoacrylate polymers such as dermabond and like) or non-biodegradable polymers (such as silicon or ultra-high-molecular-weight polyethylene). Silicon, for example, can be used for soft tissues grafts, and UHMWPE can be used for bone grafts.

Step 5: Cross-Linking

The skilled artisan will recognize that a variety of cross-linkers and/or cross-linking methods can be used. For example, UV, chemical or gaseous cross-linking can be used, such as after the plastination protocol is complete, or slow cross-linking can be used, such as during the plastination protocol. For example, the cross-linker can be mixed with the polymer (for example, alginate, calcium carbonate, and GDL).

### Alternative Embodiments

If the tissue is too dense after the plastination process, such as too dense for cell seeding or too dense for a patient's own cells to migrate through the graft, embodiments can comprise utilizing higher concentration of water mixed with the polymer bath, less cross-linking density, and/or create holes or channels (such as mechanically, laser etched, chemically, light, and the like).

Purposes and Uses:

I) Mechanical:

Currently one of the major problems with decellularized matrices in the medical fields is reduced mechanical properties. By applying plastination to decellularized soft tissues, mechanical properties would be greatly modified (i.e., improved). For example, the polymers themselves could self-cross-link with the extracellular matrix, or cross-linking the extracellular matrix to the polymers or cross-linking the polymers to itself would allow for modification of mechanical properties. For example, more crosslinking can result in higher mechanical properties, such as young modulus. Polymers that act as weeping lubrications could also be used in decellularized cartilage or like materials such as joints or discs. Weeping lubricants refers to fluid that is squeezed out mechanically from cartilage or similar implants to maintain a layer of fluid on the cartilage or implant surface, so as to reduce friction and lubricate joints.

II) Cell Seeding:

A hurdle within tissue engineering is delivering cells throughout the entire material or scaffold. Currently, some inject the cells into particular spots, create the material with cells before cross-linking for a physical encapsulation within the material (material limited) and push the cells through via fluid devices, seed on surface and allow for self-migration. Embodiments as described herein will utilize plastination for cell seeding, which will allow for cells to be seeded deeply and potentially evenly into the tissues. Such embodiments will be useful for materials that cannot be created premixed with cells, for physical encapsulation or when using whole organ decellularized scaffolds, such as lung, heart liver, pancreas.

In whole-organ biologically-derived scaffolds, for example, a common method of cellular seeding is to wash cells through the decellularized vessel networks or airways, and relying on self-migration of the cells to get into the walls and deeper parts of the whole organ scaffold. As described herein, by infusing and submerging the whole organ with polymers that have cells mixed therein, a substantially uniform distribution and thorough cellular seeding of the biologically derived scaffold will occur, even within the vessel walls and deeper of the whole organ scaffold. Vacuum pressure and duration, temperature process and solvents will be cell dependent.

III) Cell Migration/Differentiation:

Different types of polymers could be utilized to encourage different types of cell growths. For example, alginate, can encourage neurospheres to different into proportionally different subsets of cells than would agarose, agar or gellan gum. Embodiments herein capitalize on these innate properties of different polymers to encourage different cells or subset thereof to grow and/or differentiate without the use of drugs or exogenous growth factors.

### Example 2

Pelvic organ prolapse (POP) is a common condition in women caused by loss of pelvic floor tissue support, causing organs to herniate into the vaginal lumen or anus. POP symptoms, like incontinence, frequent urinary tract infections, difficult bowel movements, bleeding, pressure, and pain, can have significant negative impacts on women's health, daily living, and quality of life, especially impacting body image, family relationships, and sexual health¹⁻³. POP affects 33% to 50% of all women⁴⁻⁶, potentially affecting over 1 billion women worldwide⁷. Between 3% and 11% of all women have severe symptomatic POP^{4,6,28}. In the United States, over 300,000 POP surgeries are performed annually for symptomatic POP⁸. Surgical options for treating POP include synthetic mesh implants and acellular biologic grafts (ABGs). Synthetic mesh implants carry significant safety risks, including chronic infection, nerve and tissue damage, genital tearing, and other⁸⁻¹³. Current ABG products have fewer, usually less severe complications than synthetic mesh, but have higher recurrence rates and can have complications including infection, graft mechanical failure, and host-integration failure¹⁴⁻²². Despite the complications of current commercially available products for POP, surgeons still support their usage because synthetic mesh and ABG products have higher cure rates than traditional native tissue surgical methods, which fail within 2 years for 40% of women²³⁻²⁵. Given the negative effects of untreated POP and the risks involved in POP surgical treatment, POP represents an area of significant unmet medical need that is desperate for a safe and effective solution.

A new type of ABG for POP surgeries is being developed. Through tissue engineering methods, the graft according to the invention will have enhanced mechanical properties and will serve as a drug delivery device for delivering various therapeutic agents, such as pain medication, antibiotics, anti-infectives, combinations of such. This graft will address POP's unmet medical need, as it should be a safer, more effective product than synthetic mesh implants and ABGs currently on the market. POP has non-surgical and surgical treatment options. Non-surgical options (Kegel exercises, plastic pessaries, etc.) have limited efficacy for patients with severe POP. Surgical options include using native tissue, synthetic mesh implants, and ABGs for repair of pelvic floor support. Over 40% of vaginal POP surgeries using native tissue will fail within two years^{24, 25}. Synthetic mesh implants were created in response to high failure rates found with native tissue repairs^{13, 30}. These are most commonly made of non-absorbable polypropylene⁸. ABGs were developed as an alternative to the polypropylene meshes³⁰. ABGs are naturally derived from porcine or bovine dermal matrix that was made acellular and non-immunogenic through the process of decellularization. ABGs are ideal because they retain the native microenvironment of the extracellular matrix (ECM), which promotes host cell repopulation of the graft and, thus, complete integration of the graft with host tissue. For example, some transvaginal POP surgeries use the patient's native tissues. Native tissue repairs have discouraging rates of failure—40% of native tissue repairs fail within 2 years—and many surgeons prefer “reinforced or augmented repairs” using the two options below^{24, 25, 47-49}. Thus, ABGs are being explored as safer alternatives to mesh³⁰. Clinicians are currently using Cook Medical's Surgisis® BioDesign™ anterior pelvic floor graft and Acell, Inc.'s MatriStem™ pelvic floor matrix¹⁴. ABGs not only repair POP, but also offer a scaffold for the regeneration of the patient's tissue. To date, every ABG has shown higher recurrence rates than the synthetic meshes, but with fewer complications¹⁴.

For example, mesh implants can be made of a variety of materials, most commonly non-absorbable polypropylene⁸. Serious concerns about the safety of synthetic vaginal meshes have been raised. Currently available synthetic meshes and ABGs both have drawbacks. Synthetic mesh is the largest mass tort since asbestos, based on number of lawsuits (women have filed over 104,000 lawsuits in US federal court³¹). Several high-profile lawsuits have been filed over the use of synthetic meshes in POP surgeries, with women suffering serious side effects including death, chronic infection (e.g., sepsis), bleeding, nerve and tissue damage, perforated organs, vaginal scarring, genital tearing, pain during sex, chronic abdominal pain, and psychological distress that accompanies these physical side effects^{9, 13}. An Australian Senate inquiry, in consultation with the Medical Board of Australia, determined that vaginal mesh usage for POP caused “historic agony and pain” that had a “devastating impact” on women's health, careers, and relationships^{9, 10, 32}. In a high-profile US case in March 2018, Johnson & Johnson's Ethicon subsidiary lost a $35 million lawsuit for concealing failure, injury, and complication rates from patients. The suit claimed Ethicon's mesh product, Prolift, “caused severe and irreversible injuries, conditions, and damage to a significant number of women”³³. Some companies have opted to settle out of court rather than go on public trial. Endo International, Inc., had a $830 million settlement in 2014 and an additional $400 million settlement later that same year³⁴.

The Food and Drug Administration (FDA) issued a Public Health Notification in 2008 about the risks of using synthetic vaginal mesh for POP¹². By 2011, the FDA determined that serious adverse events associated with surgical mesh in POP are not rare and issued an update on the mesh's safety and efficacy. The FDA ultimately decided that the safety of surgical mesh for POP is not well established. In 2016, the FDA issued a final order reclassifying the meshes as Class III devices, requiring more extensive clinical trial data¹¹. In 2017, the Australian Therapeutic Goods Administration cancelled the approval of several types of surgical mesh in POP surgeries, effectively banning the products³⁵. The National Institute for Health and Clinical Excellence in the United Kingdom issued a guidance statement that acts a de facto ban on vaginal mesh in POP surgeries³⁶. Surgical meshes are banned in all pelvic operations in New Zealand, not just vaginal POP³⁶.

In POP, the vagina and supportive soft tissues are compromised both structurally and functionally, with these tissues showing disorganized and atrophied smooth muscle and altered collagen and elastin content^{30, 55-59}. As described herein, physicians are turning to ABGs as safer options for POP repair over the use of synthetic meshes.

ABGs are tissues, such as skin or tendon, that are recovered from a donor (human or animal) and processed to remove cells and immunogens. ECM proteins (e.g. collagen, elastin), which largely comprise the architecture of most tissues, are retained during decellularization. This process essentially generates a cell- and DNA-free three-dimensional graft that is amenable to host cell repopulation and remodeling during the course of healing.

Non-autologous surgical material that can be used with the graft of the invention (1) provides structural support, (2) fosters the body's natural wound-healing process, (3) allows for ingrowth, and (4) fully integrates with host tissue in time. ABGs are superior to synthetic grafts in that they preserve, as oppose to recreate, the native matrix microenvironment, retaining the unique and complex matrix composition of the tissue^{20, 60}. Preserving these complex properties promotes efficient repopulation and functional re-establishment of cells and blood supply within the ABGs, leading to their increased success over synthetic grafts. For over 30 years, ABGs have been used in surgeries for breast reconstruction, abdominal hernia repair, burn wound healing, urethra repair, tendon repair, and diabetic ulcer/chronic wound repair^{61, 62}. Commercially available ABGs, like AlloDerm, DermACELL, and FlexHD, are considered medically necessary for breast reconstructions, abdominal surgical repairs, and burns, along with other indications.

Despite the advantages of ABGs, there is significant room for improvement in ABGs for all indications. Meta-analyses on large clinical studies for ABG use in breast reconstructions and hernia repairs show ABGs have relatively high complication rates, estimated at 10-23%³⁷⁻⁴¹. Specific complications in patients with ABGs are most commonly attributed to infection (cellulitis), seroma formation, and necrosis^{15,16}. Closer analyses into ABG failures show reduced vascularization, poor mechanical integrity, failure to integrate (resulting in scarring), and immune rejection¹⁷⁻²².

To improve clinical outcomes for POP procedures and to develop new capabilities of ABGs for wider applications in regenerative medicine, the ABGs according to the invention will be impregnated with biodegradable polymers that can provide enhanced mechanical properties and local, sustained release of drugs. Without being bound by theory, polymer impregnation of an ABG will itself enhance the mechanical properties of the graft, allowing it to be more durable than a non-impregnated graft and thus prevent graft failure due to mechanical forces. Infection, commonly a result of opportunistic bacteria, could be offset through sustained, local antibiotic release from the polymer, while the addition of anti-inflammatory and analgesic agents could reduce local inflammation and decrease pain at the surgical site. These additive properties will increase graft acceptance and host-integration, lessening the likelihood of complications and easing patient recovery.

The innovation as described herein is the adaptation of tissue plastination techniques⁶³⁻⁶⁶ for mechanically strengthening ABGs and for sustained and localized delivery of drugs from these grafts. Polymer embedded grafts have been described previously; however, these are usually a combination of synthetic or natural polymers and fabricated collagen matrices⁶⁷; not naturally occurring ABGs. The inventors' method will force-impregnate polymers into the dense, native three-dimensional environment of an ABG, allowing for enhanced mechanical properties of the graft while retaining the morphological complexity of the endogenous ECM. Maintenance of the natural collagen fiber density and alignment within polymer-impregnated grafts will further provide support for robust recellularization and wound healing^{67, 68}.

Rationale.

There are three main aspects to the polymer impregnation of ABGs as described herein: (1) decellularization of intact tissue, (2) plastination-based impregnation of natural polymers, and (3) drug release from polymers. In a first instance, aspects (1) and (2) as described will be integrated. In a second instance, the inventors will then incorporate (3) drug release.

(1) Decellularization:

Decellularization techniques for whole organ regeneration of lung have been optimized in rat and non-human primate (NHP) models^{69,70}, and have now successfully been employed on human skin. With modification of the decellularization process, feasibility of decellularizing dermal matrices has now been demonstrated⁷¹. The decellularization process meets previously defined criteria for generating non-immunogenic acellular tissue post decellularization^{71, 72}. This decellularization method is covered under US patent application no. US 2018/0015204 A1²⁶, which is hereby incorporated by reference in its entirety.

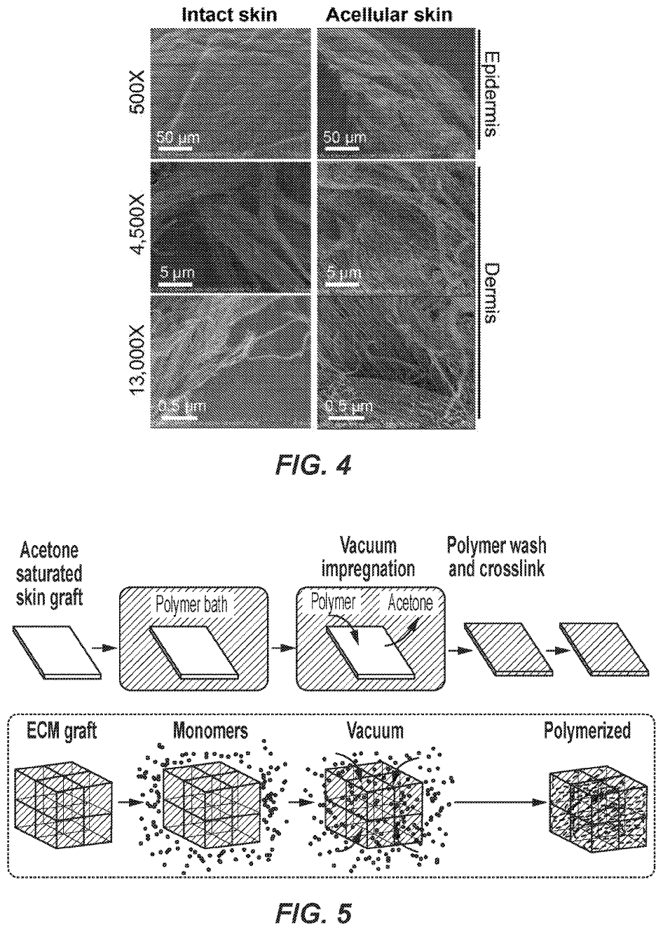

The decellularization process has been characterized on NHP dermis for the retention of ECM components collagen, glycosaminoglycans (GAGs), and elastin⁷¹. Briefly, collagens (types-I and -III) are major ECM components of skin, GAGs are hydrophilic polysaccharides that provide impact retention to the ECM, and elastin fibers are an essential component for skin elasticity. The inventors confirmed preservation of collagen and GAG content to levels similar to intact dermis, and detected a decrease in elastin levels⁷¹. A decrease in elastin is highly common in decellularized tissue^{73, 74}; however, the decreased elastin levels measured are sufficient for the maintenance of native-like mechanical properties^{73, 74}. The levels of retained ECM components from our decellularized NHP dermal matrix are similar to data from other epithelial tissue such as lung from rhesus macaque and rat, and skin from pig⁷³⁻⁷⁶. Furthermore, the decellularized dermis supported cell proliferation (>65%), cell migration, and did not significantly induce apoptosis (<1.5%)⁷¹. Additionally, the inventors evaluated ECM fiber structure of decellularized NHP dermis with scanning electron cryomicroscopy and found the integrity of collagen bundles and fibers were maintained in the dermal and epidermal layers, similar to intact dermis (FIG. 4). The decellularized dermis is devoid of immunogenic levels of genomic material and retains its overall structure on the micro- and macroscale. These data demonstrate that the decellularization process is appropriately optimized for skin, creating an acellular matrix that retains structural elements.

(2) Plastination:

Gunther von Hagens popularized plastination in the 1970s and the process has since been used to preserve surgical or autopsy tissue samples for teaching, histology, court evidence, and whole organism preservation⁶⁴. The process is well-understood⁶³⁻⁶⁶ with the most characterized method of polymer impregnation for human tissue being the S10 Technique^{65, 66}. In brief, this technique dehydrates fixed whole organs or organ slices through controlled incubations in organic solvents, including ethanol and acetone. Upon application of a vacuum, the acetone within a tissue undergoes a phase transition from liquid to gas, creating a negative pressure within the tissue that drives bath solutions, such as polymers, directly into the graft. This technique has been widely used with a range of polymers of various molecular sizes and charges, including silicones, polyesters, and resins⁷⁷⁻⁸¹. To date, plastination has not been used for the purpose of impregnating ABGs with polymers for in vivo use or in any therapeutic application.

Changes to the standard S10 Plastination Technique will be made in order to impregnate ABGs with polymers for in vivo use as well as for therapeutic applications. The inventors will use the decellularization process described above to generate a native, non-crosslinked, ABG. After dehydration of the ABG through successive incubations in ethanol, the graft will be saturated with acetone. Similar to the original plastination process, the inventors will use controlled vacuum driven force impregnation²⁷ of the graft to replace the acetone in the graft with biomaterials and biocompatible biologically-derived polymers and proteins (FIG. 5).

Drug Release:

Polymers, including biodegradable polymers, have long been used in drug delivery systems as carriers⁸². The inventors will utilize natural polymers that are amenable to drug encapsulation for localized drug delivery at sites of graft placement. The inventors will leverage knowledge and methods of dermal delivery and sustained-release of drugs that is well known in the art to develop a method to provide sustained drug release within regenerating tissue. This approach avoids systemic effects of current methods and provides a higher therapeutic dose at the relevant site of drug action. Articles have published relating to biodegradable polymers used to encapsulate and release growth factors in a controlled, sustained manner⁸³ (incorporated by reference in its entirety). This study used a novel alginate construct as a multi-functional tissue scaffold for central nervous system repair that delivered a brain-derived neurotrophic factor (Neurotrophin-3)⁸³. In the polymer-impregnated ABG program described herein, the inventors will use similar materials and methods to physically encapsulate antibiotics, analgesics, and/or anti-inflammatory agents in the polymers selected, to address issues with infection, pain, and host hyperinflammatory response.

Study.

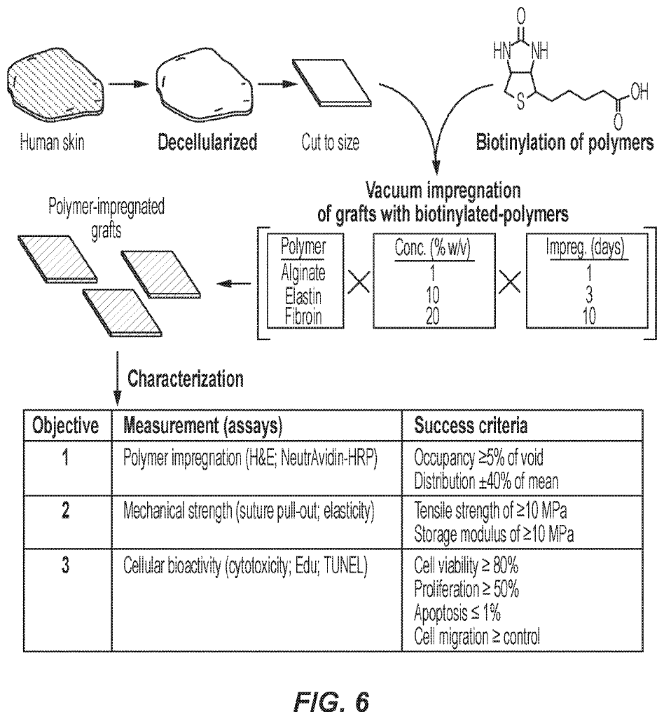

The inventors are developing a method to impregnate an ABG with biodegradable polymers that have well-characterized, robust mechanical properties and are amenable to the encapsulation of drugs for local and controlled delivery of compounds at engraftment sites. A polymer-impregnated graft will have enhanced mechanical properties, enabling it to be more durable than a non-impregnated graft and will decrease the likelihood of complications associated with mechanical failure. Controlled antibiotic release from these polymers at engraftment sites can have the potential to stem complications with infection and release of anti-inflammatory agents can immediately quell inflammation, allowing for increased likelihood of graft acceptance/integration with the host. Creation of a bioactive polymer-impregnated ABG requires complete decellularization of biologically-derived intact tissue, physical encapsulation of antibiotic and anti-inflammatory agents in a biodegradable polymer, and impregnation of the decellularized skin with the drug-loaded polymer. The inventors will focus efforts on developing and optimizing a novel polymer impregnation method based on tissue plastination techniques. The inventors will then analyze the polymer-impregnated ABGs' properties (FIG. 6). To that end, the objectives are:

Objective 1. Determine which Parameters Optimize Polymer Impregnation of ABGs.

The inventors will develop methods for impregnating acellular skin grafts with three biodegradable polymers: alginate, elastin, and silk fibroin. The three polymers will be tested at three concentrations each and three vacuum-impregnation incubation times will also be assessed. For each combination of parameters, the inventors will generate test grafts in triplicate. These grafts will be histologically analyzed for polymer penetration and distribution. The inventors will subsequently determine specific conditions that produce a polymer occupancy in the graft that is >5% of the graft void/interstitial space and have a polymer distribution such that normalized polymer occupancy is ±40% of the mean (for description, see Experimental Approach). Grafts that meet these success criteria will advance to Objectives 2 and 3 described herein.

The inventors will develop methods for impregnating acellular skin grafts with each of three biodegradable polymers: alginate, elastin, and silk fibroin. These well-characterized polymers were chosen for their biocompatibility, tunable mechanical properties, small and relatively simple monomeric form, ease of crosslinking to generate polymeric forms, biologically-derived nature, and proven use in biomedical applications. By impregnating ABGs with these polymers, the inventors can manipulate and enhance the mechanical properties of ABGs. In principle, the impregnated polymer can occupy a percentage of the void/interstitial space within the ECM and can crosslink onto itself and/or the ECM. Because these polymers readily crosslink through chemical (pH, Ca2+) or physical (temperature, sonication) mechanisms, crosslinking can occur rapidly and efficiently without regard for graft size. Non-enzyme-based crosslinking circumvents limitations with enzyme diffusion and sustained activity within the density of the graft. Additional reasons for selecting these three polymers are as follows:

(a) Alginate:

Alginate (algin, alginic acid) is a natural polymer isolated from brown seaweed and is an FDA-approved polymer generally regarded as safe (GRAS). Alginate has been widely used in regenerative medicine and drug delivery, as it is histocompatible for human use and has minimal or negligible cytotoxicity⁸⁷⁻⁹⁰. As described in this section, alginate has been used as a tissue scaffold for drug release⁸³. Alginate can be induced to form highly cross-linked hydrogels with multivalent cations (e.g. Ca²⁺)⁸³.

(b) Elastin:

The inventors have previously characterized a 69% reduction in elastin content in NHP acellular dermis from native dermis (n=3, p<0.01)⁷¹. These findings were similar to data from rhesus macaque lung, rat lung, and porcine dermis: elastin decrease during the decellularization process is a widely observed and characterized feature of detergent-based decellularization methods⁷³⁻⁷⁶. Recent studies looking at the addition of insoluble elastin in combination with collagen in a biomimetic cardiovascular tissue scaffold found that elastin markedly altered the mechanical and biological properties of the scaffold, reducing the specific tensile and compressive moduli without negatively affecting pore size or porosity⁹¹. Tropoelastin, the monomeric form of elastin, can be induced to crosslink with itself or collagen. The most commonly used elastin is α-elastin, which can be crosslinked by heat, repetitive sonication⁹¹, or pH change⁹².

(c) Silk Fibroin:

Silk is primarily composed of two proteins, fibroin and sericin. Sericin may trigger an immune response, but fibroin does not⁹³. Silk has recently been explored as a drug delivery vehicle, drug stabilizer, and biological scaffold for tissue engineering⁹³ and is an FDA approved biomaterial. Silk is of interest to biomedical engineers due to its favorable biocompatibility properties and unique mechanical attributes, including a well-defined nano- and micro-scale structure hierarchy, biocompatibility, noninflammatory by-products, and sterilization method compatibility⁹³⁻⁹⁵. The FDA has approved a variety of silk products for in vivo use, including sutures and scaffolds (e.g. Seri® Surgical Scaffold). Silk can be induced to crosslink with itself or collagen by heat or repetitive sonication⁹³.

Experimental Approach:

Polymer impregnation will be performed as described herein²⁷. FIG. 6 shows the work flow. The inventors will first decellularize human donor cadaver skin (2-3 mm thick) to serve as the model ABG. All tissue is obtained through AATB-accredited tissue banks from deceased donors. Following decellularization and prior to polymer impregnation, ABGs will be cut into 1 cm×1 cm squares. In addition, all polymers will be biotinylated via standard carbodiimide-based crosslinking to enable polymer detection within ABGs. Biotinylation of the proposed polymers is not expected to interfere with their ability to form polymers or have a significant effect on their physical properties. Biotin is a small 244 Da molecule that is widely used in conjugating proteins and has already been used with alginate⁹⁶ and fibroin⁹⁷ and shown not to alter polymer properties.

During impregnation, the ABG will be gradually dehydrated through stepwise incubations with ethanol before complete saturation with the organic solvent, acetone. The acetone-saturated ABG will be placed with a water solubilized polymer bath solution and subject to a low vacuum for different lengths of time at 4° C. Since polymerization increases under warmer temperatures, we will perform impregnations at 4° C. to ensure polymer entry into the ABG as a monomer before polymerization. For each polymer, we will test three concentrations: low (1% weight/volume), normal (10% weight/volume), and high (20% weight/volume). The amount of time needed for the vacuum-impregnation step can vary widely, depending on the thickness and complexity of the tissue being impregnated. Time frames longer than one day may be necessary at 4° C., thus the inventors will be testing 1, 3, and 10-day incubation periods.

The goal of this step is to penetrate the polymer into and throughout the ABG. The inventors will use a full factorial Design of Experiments approach to optimize polymer concentration, impregnation incubation time, and incubation temperature. For each impregnation condition, the inventors will generate impregnated grafts in triplicate for evaluation of impregnation efficacy. A total of 27 possible parameter combinations (three polymers; three concentrations; three times) will thus require generation of 81 grafts.

Samples will be submitted for histological and immunohistochemical (IHC) analysis to measure polymer occupancy and distribution within the ABG. Polymer occupancy is defined as the polymer signal area (as measured by NeutrAvidin-HRP staining of biotinylated polymers) divided by the total ABG area. Because skin is stratified, the ECM density is not uniform across its thickness (epidermis to hypodermis). Without being bound by theory, variability in polymer distribution across the thickness of the ABG. In order to measure polymer distribution, ABG area will be discretized into 500 μm×500 μm regions, normalized polymer occupancy will be calculated for each region, and then the distribution of normalized polymer occupancy throughout the graft analyzed for deviation from the mean. Normalized polymer occupancy is the polymer occupancy (i.e. % area polymer) divided by the interstitial space occupancy (i.e. % area void) prior to polymer impregnation. The interstitial space prior to impregnation will be measured directly from H&E stained sections (subtracting the ECM area from the total ABG area). The inventors are normalizing polymer occupancy after impregnation to interstitial space occupancy prior to impregnation because the amount of polymer that enters a region of the ABG is directly related to the amount of void space that existed prior to impregnation.

Conditions will be determined that produce a polymer occupancy in the graft that is >5% of the graft area and have a polymer distribution such that normalized polymer occupancy is ±40% of the mean. Without being bound by theory, a 5% increase of material within the graft will be sufficient to alter the mechanical properties. Without being bound by theory, normalized polymer occupancy will be within 40% of the mean based upon a Monte Carlo analysis for a discretized graft in which ECM occupancy and polymer occupancy for each region that were randomly assigned within the ranges of 20-50% and 1-40%, respectively. Only grafts meeting these parameters will be used.

Objective 2. Characterize the Mechanical Properties of the Polymer-Impregnated ABGs.

The inventors will test all polymer-impregnated acellular skin grafts that meet criteria described herein. The inventors will correlate mechanical properties to impregnation method. Tests include tensile testing, ball-burst testing, and suture pull-out testing. The inventors will determine which polymers and conditions optimize graft properties. For example, a polymer-permeated graft should have a Young's modulus and tensile strength≥10 MPa, which is greater than that of existing non-polymer-impregnated ABGs⁸⁴⁻⁸⁶.

Polymer impregnation of an ABG will enhance the mechanical properties of the graft, enabling it to be more durable than a non-impregnated graft. Without being bound by theory, a polymer-impregnated ABG should have a Young's modulus and tensile strength of ≥10 MPa, based on literature values from excised and in vivo human skin as well as values for existing non-polymer-impregnated ABGs^{84-86, 98, 99}. The Examiners will examine those grafts that meet the polymer impregnation requirements described herein, and only those grafts with favorable characteristics will be used in the studies described below. If multiple time points for a given polymer/concentration pair advance, only the test graft with the greatest polymer occupancy will advance. Therefore, a maximum of nine parameter combinations (in triplicate, for n=27 grafts) will advance into testing described in this section. The inventors will correlate mechanical properties with impregnation methodology. Data collected will also inform efforts to develop tunable, personalized grafts with different mechanical properties. Mechanical properties will be measured by uniaxial tensile, ball burst, and suture pull-out testing. Uniaxial tensile testing measures properties like Young's modulus (longitudinal stress:strain ratio to measure ability to withstand lengthwise tension or compression), ultimate tensile strength, yield strength, elongation, and Poisson's ratio. Ball burst testing is used to measure the resistance of a material to deformation and failure. Suture pull-out testing is commonly used to measure the resistance of a material, like surgical grafts and mesh, to failure at suture tie-in points.

Experimental Approach:

Uniaxial tensile, ball burst, and suture pull-out testing will be performed by Exponent, Inc. (Philadelphia, Pa.). For mechanical testing, polymer-impregnated grafts will be 6.4 cm×1 cm as needed for instrument fitting. Although larger than the grafts used in Objective 1, since graft thickness will be consistent, the inventors expect force-driven diffusion of polymer through the grafts to be consistent despite surface area differences. Commercially available ABGs will be included as controls for comparison.

(a) Unaxial Testing:

These tests will be performed according to ASTM D638¹⁰⁰. Samples will be cut into a dog bone shape and the sample will be tested with a video extensometer to track strain. The elastic moduli, elongation at failure, and ultimate tensile strength will be determined.

(b) Ball Burst Testing:

These tests will be performed according to ASTM D6797-15¹⁰¹ and Freytes et al¹⁰². Samples will be cut to 2×2 cm and ruptured in a probe ball burst pressure test with a 5.56 mm diameter probe and a 9.75 mm diameter clearance hole at a rate of 25.4 mm/min.

(c) Suture Pull-Out Testing:

Suture pull-out testing will be performed following ASTM F543¹⁰³. A suture will be passed through the graft once, and the loose ends will be looped and tied around a suture pull-out fixture. The skin graft will be stationary, gripped to the base of the load frame such that the direction of the suture's applied load will be parallel to the graft. A tensile load will be applied to the samples at 5 mm/min until failure occurs. The axial pullout strength of the test specimen will be determined.

If the above tests prove to be ineffective for determining the mechanical properties of the ABGs due to limited test conditions (i.e. temperature, pre-conditioning, strain rate) or suitability of tests for the material (i.e. elastic, viscoelastic, asymmetry), the inventors will use dynamic mechanical analysis (DMA). DMA is used to measure polymer viscoelasticity by determining the complex modulus (stress:strain ratio under vibratory conditions), Young's modulus, and ultimate tensile strength. DMA is an alternative to uniaxial tensile and ball burst testing. If needed, a frequency sweep will be conducted on one specimen per sample at a fixed 20° C. temperature and varied frequencies between −0.1 Hz and 10 Hz, to determine the Young's, dynamic storage, and loss moduli, tensile strength, and tan(δ) as a function of frequency.

The inventors will determine which polymers and conditions optimize graft properties. The polymer-permeated graft should have a Young's modulus and tensile strength of ≥10 MPa, which is greater than that of existing non-polymer-impregnated ABGs⁸³⁻⁸⁵.

Objective 3. Characterize the In Vitro Bioactivity of the Polymer-Impregnated ABGs.

The inventors will measure the bioactivity of the polymer-impregnated acellular skin grafts that met the above-indicated criteria by seeding grafts in vitro with human primary uterine fibroblasts and then determining percent cell viability, proliferation, and apoptosis according to methods known in the art. The inventors will also assess the ability of cells to migrate into the graft. The inventors will determine which polymers and conditions optimize graft properties, for example, a polymer-permeated graft that supports≥80% viable cells and ≥50% proliferative cells.

A polymer-impregnated ABG, such as the graft according to the invention, that is bioactive and supports cell migration, is essential for eventual clinical use. The inventors will examine those grafts meeting the requirements and only those grafts with favorable characteristics will be used in the studies described below. A maximum of nine parameter combinations (in triplicate, for n=27 grafts) will advance into testing. This testing steps uses human cells in combination with the graft of the invention.

The inventors will measure the bioactivity of the polymer-impregnated grafts by seeding grafts in vitro with human primary uterine fibroblasts and then assessing cell proliferation, apoptosis, and migration. Similar assays were previously performed after seeding NHP bone marrow-derived mesenchymal stem cells (BMSCs) onto decellularized NHP dermis⁷¹. These data showed less than 1% of cells being apoptotic and approximately 65% of the cell population remaining proliferative during the entirety of the study, with no observed changes between days⁷¹. Cell viability staining will be conducted. Microscopy work will then be performed. Ki-67, EdU, and TUNEL staining and image capture will subsequently be carried out. For all of the bioactivity assays, statistical significance will be determined with two-tailed ANOVA with α=0.05. Numerical results will be given as average±standard error of the mean.

Experimental Approach:

Acellular grafts will be pre-conditioned with cell growth media in a cell culture incubator under standard mammalian cell culture conditions (37° C./5% carbon dioxide) for 30 minutes prior to seeding. Each graft will be 1 cm×1 cm and seeded with 1×10⁶ primary uterine fibroblasts; each graft will be run in triplicate. This density should provide sufficient infiltration of the graft so as to permit sufficient cell presence within the graft as well as good single cell resolution without a high background signal due to too many cells. The seeded grafts will be cultured for one and two weeks prior to analysis.

For all stains, each seeded graft will be cut to 5 mm×5 mm pieces to avoid insufficient antibody and dye penetration into the tissue. Cell viability, proliferation, and apoptosis will be measured via fluorescence microscopy and quantified using ImageJ software. Measurements will be taken from 5 random fields of view per sample for each bioactivity assay and background subtracted. Out-of-focused signals of cells not in the focal plane will be excluded. Seeded grafts will be assessed as follows:

(a) Cell Viability:

Viability will be measured within the graft by calcein-AM and ethidium homodimer-1 staining (Thermo; LIVE/DEAD assay). Calcein-AM stains green living cells with active esterase activity, while ethidium homodimer-1 stains red the nuclei of cells with membrane integrity loss.

(b) Proliferation:

Cell proliferation will be measured through Ki-67 staining and EdU (5-ethynyl-2′-deoxyuridine) incorporation. IHC will be performed on sections of seeded grafts with anti-Ki67 (Cell Signaling, Mouse) and Alexa Fluor 488 conjugated secondary staining. The Click-iT EdU assay (Invitrogen) will label mitotically active cells through incorporation of fluorescently label EdU.

(c) Apoptosis:

Apoptosis will be assessed by the TdT-mediated dUTP Nick-end Labeling (TUNEL) assay (In Situ Cell Death Detection Kit—Fluorescein, Roche) as previously described^{71, 73}. Samples treated with labeled solution without enzyme will be included as negative controls while samples treated with DNase I prior to TUNEL staining will serve as positive controls. All samples will be stained with DAPI after TUNEL staining.

(d) Cell Migration:

Using the slide images captured in the histology work described above, we will assess the ability of cells to migrate into the polymer-impregnated ABGs as compared to non-polymer treated ABGs. We will count relative number of cells, as indicated by stained nuclei, within each graft.

The cell migration study will quantify the number of cells per field as a function of distance from the graft edge in order to generate a “migration” value. However, fibroblasts seeded on a single side of the graft will presumably migrate into the graft on that single side. The majority of the migrating cells will be from the area where the graft makes direct contact with the cell culture plate, and distance migrated will be skewed depending on the original position of the migrating cells. For this reason, the number of cells migrated will only be counted. The inventors will be able to make more quantitative assessments of migration distance in later studies when the graft is implanted in an in vivo model.

Thus, the inventors will determine which polymers and conditions optimize graft properties, for example, the polymer-permeated graft will support>80% viable cells and >50% proliferative cells. The inventors will further select optimal parameter conditions to advance to later studies.

The inventors are developing polymer-impregnated ABGs to improve mechanical function of ABGs and deliver drugs locally in a controlled manner to enhance healing and patient outcomes. The Example herein describes efforts to produce a polymer-impregnated acellular skin graft. In a subsequent study, the inventors will mix selected drugs with the biodegradable polymer prior to graft polymer impregnation. By choosing tunable biodegradable materials that are meant to break down within the body over time, the inventors will allow for sustained drug delivery to the area surrounding the graft. The inventors will then characterize encapsulation efficacy and drug-release kinetics as well as graft genotoxicity. These activities will advance the polymer-impregnated ABG studies.

### REFERENCES FOR THIS EXAMPLE

1. Lowenstein L, Gamble T, Sanses T V, van Raalte H, Carberry C, Jakus S, Kambiss S, McAchran S, Pham T, Aschkenazi S, Hoskey K, Kenton K, Fellow's Pelvic Research N. Sexual function is related to body image perception in women with pelvic organ prolapse. J Sex Med. 2009; 6(8):2286-2291. doi: 10.1111/j.1743-6109.2009.01329.x. PubMed PMID: 19493287.
2. Fritel X, Varnoux N, Zins M, Breart G, Ringa V. Symptomatic pelvic organ prolapse at midlife, quality of life, and risk factors. Obstet Gynecol. 2009; 113(3):609-616. doi: 10.1097/AOG.0b013e3181985312. PubMed PMID: 19300324; PMCID: PMC2850374.
3. Chan S S, Cheung R Y, Yiu K W, Lee L L, Pang A W, Chung T K. Symptoms, quality of life, and factors affecting women's treatment decisions regarding pelvic organ prolapse. Int Urogynecol J. 2012; 23(8):1027-1033. doi: 10.1007/s00192-012-1698-y. PubMed PMID: 22398825.
4. Barber M D, Maher C. Epidemiology and outcome assessment of pelvic organ prolapse. Int Urogynecol J. 2013; 24(11):1783-1790. doi: 10.1007/s00192-013-2169-9. PubMed PMID: 24142054.
5. Aytan H, Ertunc D, Tok E C, Yasa O, Nazik H. Prevalence of pelvic organ prolapse and related factors in a general female population. Turk J Obstet Gynecol. 2014; 11(3):176-180. doi: 10.4274/tjod.90582. PubMed PMID: 28913013; PMCID: PMC5558330.
6. Barber M D. Pelvic organ prolapse. BMJ. 2016; 354. doi: 10.1136/bmj.i3853. 7. “World Population 2018.” from https://countrymeters.info/en/World.
8. US Food and Drug Administration. Urogynecologic Surgical Mesh: Update on the Safety and Effectiveness of Transvaginal Placement for Pelvic Organ Prolapse. 2011.
9. BBC News (2018). “Vaginal mesh implants: Australia apologises for ‘decades of pain’.” from https://www.bbc.com/news/world-australia-45806324.
10. Scott S, Branley A (2018). “Mesh implants: Government issues national apology over ‘agony and pain’ caused by device.” from https://www.abc.net.au/news/2018-10-10/mesh-implants-government-issues-apology-to-women/10355546?section=health.
11. Kux L. Effective Date of Requirement for Premarket Approval for Surgical Mesh for Transvaginal Pelvic Organ Prolapse Repair. In: Food and Drug Administration, editor. Federal Register 2016. p. 364-370.
12. Schultz D G. FDA Public Health Notification: Serious Complications Associated with Transvaginal Placement of Surgical Mesh in Repair of Pelvic Organ Prolapse and Stress Urinary Incontinence. In: Center for Devices and Radiological Health, Food and Drug Administration, editors. 2008.
13. Campbell P, Jha S, Cutner A. Vaginal mesh in prolapse surgery. The Obstetrician & Gynaecologist. 2018; 20:49-56. doi: 10.1111/tog.12454.
14. Margie Kahn. Personal Communication. 2018.
15. Weichman K E, Wilson S C, Weinstein A L, Hazen A, Levine J P, Choi M, Karp N S. The use of acellular dermal matrix in immediate two-stage tissue expander breast reconstruction. Plast Reconstr Surg. 2012; 129(5):1049-1058. doi: 10.1097/PRS.0b013e31824a2acb. PubMed PMID: 22544088.
16. Chun Y S, Verma K, Rosen H, Lipsitz S, Morris D, Kenney P, Eriksson E. Implant-based breast reconstruction using acellular dermal matrix and the risk of postoperative complications. Plast Reconstr Surg. 2010; 125(2):429-436. doi: 10.1097/PRS.0b013e3181c82d90. PubMed PMID: 20124828.
17. Vig K, Chaudhari A, Tripathi S, Dixit S, Sahu R, Pillai S, Dennis V A, Singh S R. Advances in Skin Regeneration Using Tissue Engineering. Int J Mol Sci. 2017; 18(4). doi: 10.3390/ijms18040789. PubMed PMID: 28387714; PMCID: PMC5412373.
18. MacNeil S. Progress and opportunities for tissue-engineered skin. Nature. 2007; 445(7130):874-880. doi: 10.1038/nature05664. PubMed PMID: 17314974.
19. Morris A H, Stamer D, Kyriakides T, editors. The host response to naturally-derived extracellular matrix biomaterials. Seminars in Immunology; 2017: Elsevier.
20. Shau D, Patton R, Patel S, Ward L, Guild G, 3rd. Synthetic mesh vs. allograft extensor mechanism reconstruction in total knee arthroplasty—A systematic review of the literature and meta-analysis. Knee. 2018; 25(1):2-7. doi: 10.1016/j.knee.2017.12.004. PubMed PMID: 29325835.
21. Purnell C A, Souza J M, Park E, Dumanian G A. Repair of recurrent hernia after biologic mesh failure in abdominal wall reconstruction. Am J Surg. 2014; 208(5):788-793. doi: 10.1016/j.amjsurg.2014.05.008. PubMed PMID: 25118163.
22. Macadam S A, Lennox P A. Acellular dermal matrices: Use in reconstructive and aesthetic breast surgery. Can J Plast Surg. 2012; 20(2):75-89. PubMed PMID: 23730154; PMCID: PMC3383551.
23. Future Market Insights. “Female Pelvic Implants Market: Companies to Focus on Reducing Cost of Implants to Gain Maximum Market Share: Global Industry Analysis 2012-2016 and Opportunity Assessment 2017-2027.” Market Report. 2018
24. Barber M D, Brubaker L, Burgio K L, Richter H E, Nygaard I, Weidner A C, Menefee S A, Lukacz E S, Norton P, Schaffer J, Nguyen J N, Borello-France D, Goode P S, Jakus-Waldman S, Spino C, Warren L K, Gantz M G, Meikle S F, Eunice Kennedy Shriver National Institute of Child Health, Human Development Pelvic Floor Disorders Network. Comparison of 2 transvaginal surgical approaches and perioperative behavioral therapy for apical vaginal prolapse: the OPTIMAL randomized trial. JAMA. 2014; 311(10):1023-1034. doi: 10.1001/jama.2014.1719. PubMed PMID: 24618964; PMCID: PMC4083455.
25. Siff L N, Barber M D. Native Tissue Prolapse Repairs: Comparative Effectiveness Trials. Obstet Gynecol Clin North Am. 2016; 43(1):69-81. doi: 10.1016/j.ogc.2015.10.003. PubMed PMID: 26880509.
26. Pashos N C, Bunnell B A, inventors; The Administrators of the Tulane Educational Fund, assignee. Surgical Grafts for Replacing the Nipple and Areola or Damaged Epidermis. US patent US2018/0015204A1. Filed Oct. 31, 2015. Pub. Date Jan. 18, 2018.
27. Pashos N C, Heim W M, inventors; BioAesthetics Corporation, assignee. Polymer-Permeated Grafts and Methods of Making and Using the Same. patent U.S. Provisional Patent Applicaton 62/651,485. Filed Apr. 2, 2018.
28. Transparency Market Research. “Pelvic Organ Prolapse Devices Market—Global Industry Analysis, Size, Share, Growth, Trends and Forecast 2018-2026.” Market Report. 2018
29. Economics and Statistics Administration, US Census Bureau, US Department of Commerce. Age and Sex Composition: 2010. 2011.
30. Liang R, Knight K, Easley D, Palcsey S, Abramowitch S, Moalli P A. Towards rebuilding vaginal support utilizing an extracellular matrix bioscaffold. Acta Biomater. 2017; 57:324-333. doi: 10.1016/j.actbio.2017.05.015. PubMed PMID: 28487243; PMCID: PMC5639927.
31. Pelley S (2018). “Gynecological Mesh: The Medical Device that has 100,000 Women Suing.” CBS 60 Minutes. from https://www.cbsnews.com/news/boston-scientific-gynecological-mesh-the-medical-device-that-has-100000-women-suing/.
32. Australia Co. Number of women in Australia who have had transvaginal mesh implants and related matters. 2018.
33. Perriello B (2018). “Johnson & Johnson's Ethicon loses $35 m pelvic mesh lawsuit.” from https://www.massdevice.com/johnson-johnsons-ethicon-loses-35m-pelvic-mesh-lawsuit/.
34. York M A (2018). “Ethicon, Inc. and J&J facing thousands of TVM and hernia mesh lawsuits: will they settle sooner or later?”. from https://www.masstortnexus.com/mass-torts-news/ethicon-inc-and-jj-facing-thousands-of-tvm-and-hernia-mesh-lawsuits-will-they-settle-sooner-or-later/.
35. Therapeutic Goods Administration (2017). “TGA undertakes regulatory actions after review into urogynaecological surgical mesh implants.” from https://www.tga.gov.au/alert/tga-actions-after-review-urogynaecological-surgical-mesh-implants.
36. Ismail S. Three countries ban the use of vaginal mesh in prolapse surgery. International Urogynecological Association 2018; 12(4).
37. Kim J Y, Davila A A, Persing S, Connor C M, Jovanovic B, Khan S A, Fine N, Rawlani V. A meta-analysis of human acellular dermis and submuscular tissue expander breast reconstruction. Plast Reconstr Surg. 2012; 129(1):28-41. doi: 10.1097/PRS.0b013e3182361fd6. PubMed PMID: 22186498.
38. Ho G, Nguyen T J, Shahabi A, Hwang B H, Chan L S, Wong A K. A systematic review and meta-analysis of complications associated with acellular dermal matrix-assisted breast reconstruction. Ann Plast Surg. 2012; 68(4):346-356. doi: 10.1097/SAP.0b013e31823f3cd9. PubMed PMID: 22421476.
39. Loo Y L, Kamalathevan P, Ooi P S, Mosahebi A. Comparing the Outcome of Different Biologically Derived Acellular Dermal Matrices in Implant-based Immediate Breast Reconstruction: A Meta-analysis of the Literatures. Plast Reconstr Surg Glob Open. 2018; 6(3):e1701. doi: 10.1097/GOX.0000000000001701. PubMed PMID: 29707460; PMCID: PMC5908498.
40. Trippoli S, Caccese E, Tulli G, Ipponi P, Marinai C, Messori A. Biological meshes for abdominal hernia: Lack of evidence-based recommendations for clinical use. Int J Surg. 2018; 52:278-284. doi: 10.1016/j.ijsu.2018.02.046. PubMed PMID: 29501796.
41. Slater N J, van der Kolk M, Hendriks T, van Goor H, Bleichrodt R P. Biologic grafts for ventral hernia repair: a systematic review. Am J Surg. 2013; 205(2):220-230. doi: 10.1016/j.amjsurg.2012.05.028. PubMed PMID: 23200988.
42. Lawrence J M, Lukacz E S, Nager C W, Hsu J W, Luber K M. Prevalence and co-occurrence of pelvic floor disorders in community-dwelling women. Obstet Gynecol. 2008; 111(3):678-685. doi: 10.1097/AOG.0b013e3181660c1b. PubMed PMID: 18310371.
43. Samuelsson E C, Victor F T, Tibblin G, Svardsudd K F. Signs of genital prolapse in a Swedish population of women 20 to 59 years of age and possible related factors. Am J Obstet Gynecol. 1999; 180(2 Pt 1):299-305. PubMed PMID: 9988790.
44. Wu J M, Matthews C A, Conover M M, Pate V, Jonsson Funk M. Lifetime risk of stress urinary incontinence or pelvic organ prolapse surgery. Obstet Gynecol. 2014; 123(6):1201-1206. doi: 10.1097/AOG.0000000000000286. PubMed PMID: 24807341; PMCID: PMC4174312.
45. Market Reports World. “Global Pelvic Organ Prolapse Repair Device Market Report 2017.” Market Report. 2018
46. Brown L K, Fenner D E, Berger M B, Delancey J O, Morgan D M, Patel D A, Schimpf M O. Defining patients' knowledge and perceptions of vaginal mesh surgery. Female Pelvic Med Reconstr Surg. 2013; 19(5):282-287. doi: 10.1097/SPV.0b013e31829ff765. PubMed PMID: 23982577; PMCID: PMC4102428.
47. Weber A M, Walters M D, Piedmonte M R, Ballard L A. Anterior colporrhaphy: a randomized trial of three surgical techniques. Am J Obstet Gynecol. 2001; 185(6):1299-1304; discussion 1304-1296. doi: 10.1067/mob.2001.119081. PubMed PMID: 11744900.
48. Olsen A L, Smith V J, Bergstrom J O, Colling J C, Clark A L. Epidemiology of surgically managed pelvic organ prolapse and urinary incontinence. Obstet Gynecol. 1997; 89(4):501-506. doi: 10.1016/S0029-7844(97)00058-6. PubMed PMID: 9083302.
49. Kontogiannis S, Goulimi E, Giannitsas K. Reasons for and Against Use of Non-absorbable, Synthetic Mesh During Pelvic Organ Prolapse Repair, According to the Prolapsed Compartment. Adv Ther. 2017; 33(12):2139-2149. doi: 10.1007/s12325-016-0425-3. PubMed PMID: 27757813; PMCID: PMC5126199.
50. “Pelvic Organ Prolapse Repair Device Market Analysis, Market Size, Application Analysis, Regional Outlook, Competitive Strategies, and Forecasts, 2018-2025” Market Report. Market Expertz, 2018
51. Patrick J. Culligan C S, Christa Lewis, Troy D. Abell Cost-effectiveness analysis comparing robotic sacrocolpopexy to a vaginal mesh hysteropexy for treatment of uterovaginal prolapse. Open Journal of Obstetrics and Gynecology. 2013; 3:613-620. doi: 10.4236/ojog.2013.38110.
52. “Wound Care Market and Market Share Analysis.” Market Report. Kalorama, 2017
53. “The Worldwide Market for Advanced Wound Care Products.” Market Report. Kalorama, 2018
54. Donate Life America (2018). “Tissue Donation: Everything You Need to Know.”

from https://www.donatelife.net/types-of-donation/tissue-donation/.
55. Bildircin D, Kokcu A, Celik H, Sagir D, Kefeli M. Comparison of connective tissue components in the uterine ligaments between women with and without pelvic organ prolapse. Minerva Ginecol. 2014; 66(2):201-208. PubMed PMID: 24848078.
56. Chen B, Yeh J. Alterations in connective tissue metabolism in stress incontinence and prolapse. J Urol. 2011; 186(5):1768-1772. doi: 10.1016/j.juro.2011.06.054. PubMed PMID: 21944102.
57. Meijerink A M, van Rijssel R H, van der Linden P J. Tissue composition of the vaginal wall in women with pelvic organ prolapse. Gynecol Obstet Invest. 2013; 75(1):21-27. doi: 10.1159/000341709. PubMed PMID: 23108059.
58. Boreham M K, Miller R T, Schaffer J I, Word R A. Smooth muscle myosin heavy chain and caldesmon expression in the anterior vaginal wall of women with and without pelvic organ prolapse. Am J Obstet Gynecol. 2001; 185(4):944-952. doi: 10.1067/mob.2001.117342. PubMed PMID: 11641683.
59. Boreham M K, Wai C Y, Miller R T, Schaffer J I, Word R A. Morphometric analysis of smooth muscle in the anterior vaginal wall of women with pelvic organ prolapse. Am J Obstet Gynecol. 2002; 187(1):56-63. PubMed PMID: 12114889.
60. Lintin L A, Kingsnorth A N. Mechanical failure of a lightweight polypropylene mesh. Hernia. 2014; 18(1):131-133. doi: 10.1007/s10029-012-0959-5. PubMed PMID: 22824989.
61. Jeffcoate W J, Vileikyte L, Boyko E J, Armstrong D G, Boulton A J M. Current Challenges and Opportunities in the Prevention and Management of Diabetic Foot Ulcers. Diabetes Care. 2018; 41(4):645-652. doi: 10.2337/dc17-1836. PubMed PMID: 29559450.
62. Scarritt M E, Pashos N C, Bunnell B A. A review of cellularization strategies for tissue engineering of whole organs. Front Bioeng Biotechnol. 2015; 3:43. doi: 10.3389/fbioe.2015.00043. PubMed PMID: 25870857; PMCID: PMC4378188.
63. von Hagens G, Tiedemann K, Kriz W. The current potential of plastination. Anat Embryol (Berl). 1987; 175(4):411-421. PubMed PMID: 3555158.
64. Prasad G, Karkera B, Pandit S, Desai D, Tonse R G. Preservation of Tissue by Plastination: A Review. International Journal of Advanced Health Sciences. 2015; 1(11):27-31.
65. Riederer B M. Plastination and its importance in teaching anatomy. Critical points for long-term preservation of human tissue. J Anat. 2014; 224(3):309-315. doi: 10.1111/joa.12056. PubMed PMID: 23621482; PMCID: PMC3931543.
66. Latorre R M, Garcia-Sanz M P, Moreno M, Hernandez F, Gil F, Lopez O, Ayala M D, Ramirez G, Vazquez J M, Arencibia A, Henry R W. How useful is plastination in learning anatomy? J Vet Med Educ. 2007; 34(2):172-176. PubMed PMID: 17446645.
67. Castano O, Perez-Amodio S, Navarro-Requena C, Mateos-Timoneda M A, Engel E. Instructive microenvironments in skin wound healing: Biomaterials as signal releasing platforms. Advanced drug delivery reviews. 2018; 129:95-117. Epub 2018/04/09. doi: 10.1016/j.addr.2018.03.012. PubMed PMID: 29627369.
68. He C, Yang Z, Jin Y, Qi X, Chu J, Deng X. ADM Scaffolds Generate a Pro-regenerative Microenvironment During Full-Thickness Cutaneous Wound Healing Through M2 Macrophage Polarization via Lamtor1. Frontiers in physiology. 2018; 9:657. Epub 2018/06/20. doi: 10.3389/fphys.2018.00657. PubMed PMID: 29915541; PMCID: PMC5994424.
69. Bonvillain R W, Scarritt M E, Pashos N C, Mayeux J P, Meshberger C L, Betancourt A M, Sullivan D E, Bunnell B A. Nonhuman primate lung decellularization and recellularization using a specialized large-organ bioreactor. J Vis Exp. 2013(82):e50825. doi: 10.3791/50825. PubMed PMID: 24378384; PMCID: PMC4048361.
70. Scarritt M E, Pashos N C, Motherwell J M, Eagle Z R, Burkett B J, Gregory A N, Mostany R, Weiss D J, Alvarez D F, Bunnell B A. Re-endothelialization of rat lung scaffolds through passive, gravity-driven seeding of segment-specific pulmonary endothelial cells. J Tissue Eng Regen Med. 2018; 12(2):e786-e806. doi: 10.1002/term.2382. PubMed PMID: 27943597.
71. Pashos N C, Scarritt M E, Eagle Z R, Gimble J M, Chaffin A E, Bunnell B A. Characterization of an Acellular Scaffold for a Tissue Engineering Approach to the Nipple-Areolar Complex Reconstruction. Cells Tissues Organs. 2017; 203(3):183-193. doi: 10.1159/000455070. PubMed PMID: 28125805; PMCID: PMC5973798.
72. Crapo P M, Gilbert T W, Badylak S F. An overview of tissue and whole organ decellularization processes. Biomaterials. 2011; 32(12):3233-3243. doi: 10.1016/j.biomaterials.2011.01.057. PubMed PMID: 21296410; PMCID: PMC3084613.
73. Bonvillain R W, Danchuk S, Sullivan D E, Betancourt A M, Semon J A, Eagle M E, Mayeux J P, Gregory A N, Wang G, Townley I K, Borg Z D, Weiss D J, Bunnell B A. A nonhuman primate model of lung regeneration: detergent-mediated decellularization and initial in vitro recellularization with mesenchymal stem cells. Tissue Eng Part A. 2012; 18(23-24):2437-2452. doi: 10.1089/ten.TEA.2011.0594. PubMed PMID: 22764775; PMCID: PMC3501118.
74. Scarritt M E, Bonvillain R W, Burkett B J, Wang G, Glotser E Y, Zhang Q, Sammarco M C, Betancourt A M, Sullivan D E, Bunnell B A. Hypertensive rat lungs retain hallmarks of vascular disease upon decellularization but support the growth of mesenchymal stem cells. Tissue Eng Part A. 2014; 20(9-10):1426-1443. doi: 10.1089/ten.TEA.2013.0438. PubMed PMID: 24378017; PMCID: PMC4011420.
75. Hoganson D M, Owens G E, O'Doherty E M, Bowley C M, Goldman S M, Harilal D O, Neville C M, Kronengold R T, Vacanti J P. Preserved extracellular matrix components and retained biological activity in decellularized porcine mesothelium. Biomaterials. 2010; 31(27):6934-6940. doi: 10.1016/j.biomaterials.2010.05.026. PubMed PMID: 20584548.
76. Price A P, England K A, Matson A M, Blazar B R, Panoskaltsis-Mortari A. Development of a decellularized lung bioreactor system for bioengineering the lung: the matrix reloaded. Tissue Eng Part A. 2010; 16(8):2581-2591. doi: 10.1089/ten.TEA.2009.0659. PubMed PMID: 20297903; PMCID: PMC2947435.
77. Chisholm F, Varsou O. Resin-embedded anatomical cross-sections as a teaching adjunct for medical curricula: is this technique an alternative to potting and plastination? J Anat. 2018; 233(1):98-105. doi: 10.1111/joa.12816. PubMed PMID: 29663381.
78. Ottone N E, Baptista C A C, Latorre R, Bianchi H F, Del Sol M, Fuentes R. E12 sheet plastination: Techniques and applications. Clin Anat. 2018; 31(5):742-756. doi: 10.1002/ca.23008. PubMed PMID: 29082560.
79. Rabiei A A, Esfandiary E, Hajian M, Shamosi A, Mardani M, Rashidi B, Setayeshmehr M. Plastination of decalcified bone by a new resin technique. Adv Biomed Res. 2014; 3:18. doi: 10.4103/2277-9175.124648. PubMed PMID: 24592368; PMCID: PMC3929099.
80. Soal S, Pollard M, Burland G, Lissaman R, Wafer M, Stringer M D. Rapid ultrathin slice plastination of embalmed specimens with minimal tissue loss. Clin Anat. 2010; 23(5):539-544. doi: 10.1002/ca.20972. PubMed PMID: 20235170.
81. Miklosova M, Miklos V. Plastination with silicone method S 10—monitoring and analysis causes of failure. Biomed Pap Med Fac Univ Palacky Olomouc Czech Repub. 2004; 148(2):237-238. PubMed PMID: 15744385.
82. Liechty W B, Kryscio D R, Slaughter B V, Peppas N A. Polymers for drug delivery systems. Annu Rev Chem Biomol Eng. 2010; 1:149-173. doi: 10.1146/annurev-chembioeng-073009-100847. PubMed PMID: 22432577; PMCID: PMC3438887.
83. Shanbhag M S, Lathia J D, Mughal M R, Francis N L, Pashos N, Mattson M P, Wheatley M A. Neural progenitor cells grown on hydrogel surfaces respond to the product of the transgene of encapsulated genetically engineered fibroblasts. Biomacromolecules. 2010; 11(11):2936-2943. doi: 10.1021/bm100699q. PubMed PMID: 20942395; PMCID: PMC3775902.
84. Filipe E C, Santos M, Hung J, Lee B S L, Yang N, Chan A H P, Ng M K C, Rnjak-Kovacina J, Wise S G. Rapid Endothelialization of Off-the-Shelf Small Diameter Silk Vascular Grafts. JACC Basic Transl Sci. 2018; 3(1):38-53. doi: 10.1016/j.jacbts.2017.12.003. PubMed PMID: 30062193; PMCID: PMC6058932.
85. Pierce L M, Grunlan M A, Hou Y, Baumann S S, Kuehl T J, Muir T W. Biomechanical properties of synthetic and biologic graft materials following long-term implantation in the rabbit abdomen and vagina. Am J Obstet Gynecol. 2009; 200(5):549 e541-548. doi: 10.1016/j.ajog.2008.12.041. PubMed PMID: 19285647.
86. Musculoskeletal Transplant Foundation. DermaMatrix Acellular Dermis. Comparative testing. 2012.
87. Sun J, Tan H. Alginate-Based Biomaterials for Regenerative Medicine Applications. Materials (Basel). 2013; 6(4):1285-1309. doi: 10.3390/ma6041285. PubMed PMID: 28809210; PMCID: PMC5452316.
88. McCanless J D, Jennings L K, Bumgardner J D, Cole J A, Haggard W O. Hematoma-inspired alginate/platelet releasate/CaPO4 composite: initiation of the inflammatory-mediated response associated with fracture repair in vitro and ex vivo injection delivery. J Mater Sci Mater Med. 2012; 23(8):1971-1981. doi: 10.1007/s10856-012-4672-9. PubMed PMID: 22588505.
89. de Vos P, Spasojevic M, de Haan B J, Faas M M. The association between in vivo physicochemical changes and inflammatory responses against alginate based microcapsules. Biomaterials. 2012; 33(22):5552-5559. doi: 10.1016/j.biomaterials.2012.04.039. PubMed PMID: 22560199.
90. Vanacker J, Luyckx V, Dolmans M M, Des Rieux A, Jaeger J, Van Langendonckt A, Donnez J, Amorim C A. Transplantation of an alginate-matrigel matrix containing isolated ovarian cells: first step in developing a biodegradable scaffold to transplant isolated preantral follicles and ovarian cells. Biomaterials. 2012; 33(26):6079-6085. doi: 10.1016/j.biomaterials.2012.05.015. PubMed PMID: 22658800.
91. Ryan A J, O'Brien F J. Insoluble elastin reduces collagen scaffold stiffness, improves viscoelastic properties, and induces a contractile phenotype in smooth muscle cells. Biomaterials. 2015; 73:296-307. doi: 10.1016/j.biomaterials.2015.09.003. PubMed PMID: 26431909.
92. Mithieux S M, Tu Y, Korkmaz E, Braet F, Weiss A S. In situ polymerization of tropoelastin in the absence of chemical cross-linking. Biomaterials. 2009; 30(4):431-435. doi: 10.1016/j.biomaterials.2008.10.018. PubMed PMID: 18996590.
93. Yucel T, Lovett M L, Kaplan D L. Silk-based biomaterials for sustained drug delivery. J Control Release. 2014; 190:381-397. doi: 10.1016/j.jconre1.2014.05.059. PubMed PMID: 24910193; PMCID: PMC4142080.
94. Horan R L, Toponarski I, Boepple H E, Weitzel P P, Richmond J C, Altman G H. Design and characterization of a scaffold for anterior cruciate ligament engineering. J Knee Surg. 2009; 22(1):82-92. PubMed PMID: 19216356.
95. Wang Y, Rudym D D, Walsh A, Abrahamsen L, Kim H J, Kim H S, Kirker-Head C, Kaplan D L. In vivo degradation of three-dimensional silk fibroin scaffolds. Biomaterials. 2008; 29(24-25):3415-3428. doi: 10.1016/j.biomaterials.2008.05.002. PubMed PMID: 18502501; PMCID: PMC3206261.
96. Polyak B, Geresh S, Marks R S. Synthesis and characterization of a biotin-alginate conjugate and its application in a biosensor construction. Biomacromolecules. 2004; 5(2):389-396. doi: 10.1021/bm034454a. PubMed PMID: 15002998.
97. Wang X, Kaplan D L. Functionalization of silk fibroin with NeutrAvidin and biotin. Macromol Biosci. 2011; 11(1):100-110. doi: 10.1002/mabi.201000173. PubMed PMID: 20824692.
98. Manschot J F, Brakkee A J. The measurement and modelling of the mechanical properties of human skin in vivo—I. The measurement. J Biomech. 1986; 19(7):511-515. PubMed PMID: 3745223.
99. Ni Annaidh A, Bruyere K, Destrade M, Gilchrist M D, Ottenio M. Characterization of the anisotropic mechanical properties of excised human skin. J Mech Behav Biomed Mater. 2012; 5(1):139-148. doi: 10.1016/j jmbbm.2011.08.016. PubMed PMID: 22100088.
100. American Society for Testing and Materials. ASTM D638-14, Standard Test Method for Tensile Properties of Plastics. West Conshohocken, Pa.: ASTM International; 2014.
101. American Society for Testing and Materials. ASTM D6797-15, Standard Test Method for Bursting Strength of Fabrics Constant-Rate-of-Extension (CRE) Ball Burst Test. West Conshohocken, Pa.: ASTM International; 2015.
102. Freytes D O, Rundell A E, Vande Geest J, Vorp D A, Webster T J, Badylak S F. Analytically derived material properties of multilaminated extracellular matrix devices using the ball-burst test. Biomaterials. 2005; 26(27):5518-5531. doi: 10.1016/j.biomaterials.2005.01.070. PubMed PMID: 15860208.
103. American Society for Testing and Materials. ASTM F543-17, Standard Specification and Test Methods for Metallic Medical Bone Screws. West Conshohocken, Pa.: ASTM International; 2014.

### Example 3

Acellular Nipple Areolar Complex Allografts for Use in Breast Reconstruction

There are more than 3 million breast cancer survivors living in the United States, with a significant number who had underwent mastectomies followed by breast and nipple-areolar complex (NAC) reconstructions. Currently strategies for NAC reconstruction are dependent on non-living or non-permanent techniques: tattooing, prosthetics or surgical nipple-like structures. Described herein is a tissue engineering approach that may permit a human NAC onlay graft during breast reconstruction procedures. By applying decellularization, the removal of cellular components from tissue, to an intact whole donor NAC, the extracellular matrix (ECM) structure of the NAC is preserved; thereby, creating a biologically derived scaffold for cells to repopulate and regenerate the NAC.

Non-human primate (NHP) NAC tissue were used a model for human tissues. A detergent-based decellularization method was used to derive whole-NAC scaffolds from NHP NAC tissue. In vitro characterization include: cell viability, proliferation/apoptosis, histological analysis, proteomic profile comparative analysis and material analysis. In vivo analysis of biocompatibility, vasculogensis and feasibility was evaluated using two models, murine and NHP animal models.

In the murine C57BL/6 subcutaneous implant model of biocompatibility and neovascularization, decellularized rhesus-derived NAC grafts were compared to surgery only, native mouse skin, and commercially available porcine-derived acellular dermis, over 3 weeks. To evaluate neovascularization and immune cell infiltrate, peripheral blood, weights, and the implants were collected; flow cytometry and immunohistochemistry was performed. Feasibility was conducted by a non-lethal NHP rhesus macaque onlay engraftment model, CBC/chem12, and weight was analyzed over the 6 week study, and immunochemistry was performed on each of the 20 NAC engraftments. Statistical significance was determined by two-tailed t-tests or two-way ANOVA with a Tukeyspost-hoc test. In vitro sample size: n>3; in vivo: murine n>5/group; NHP grafts n=6/timepoint

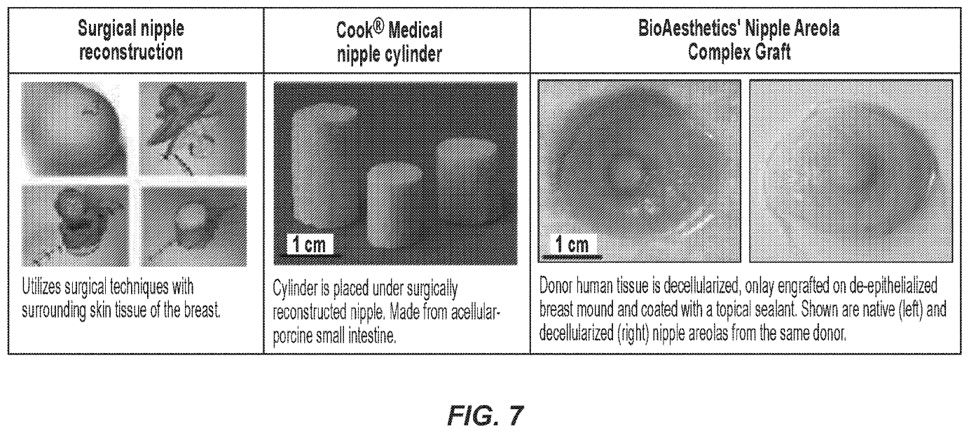

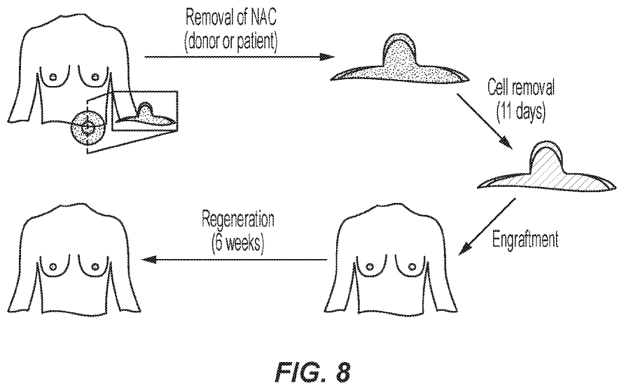

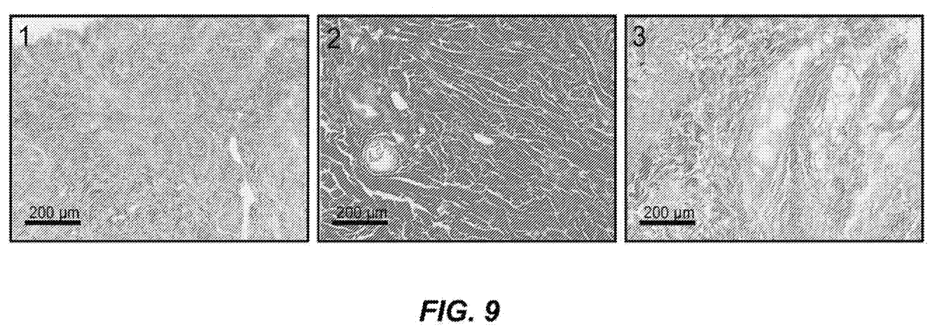

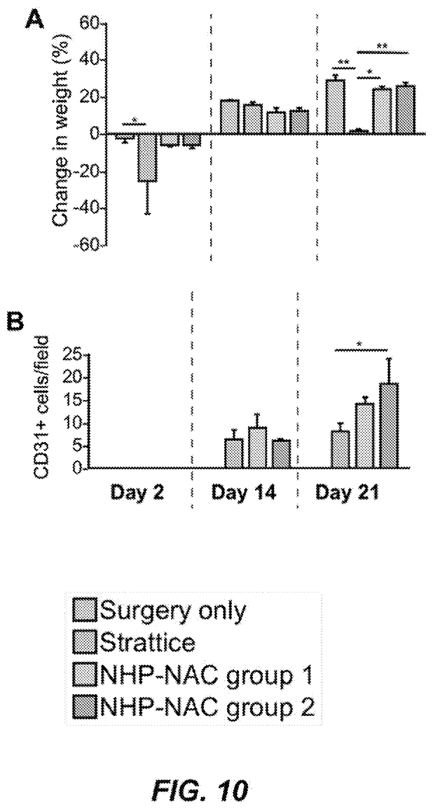

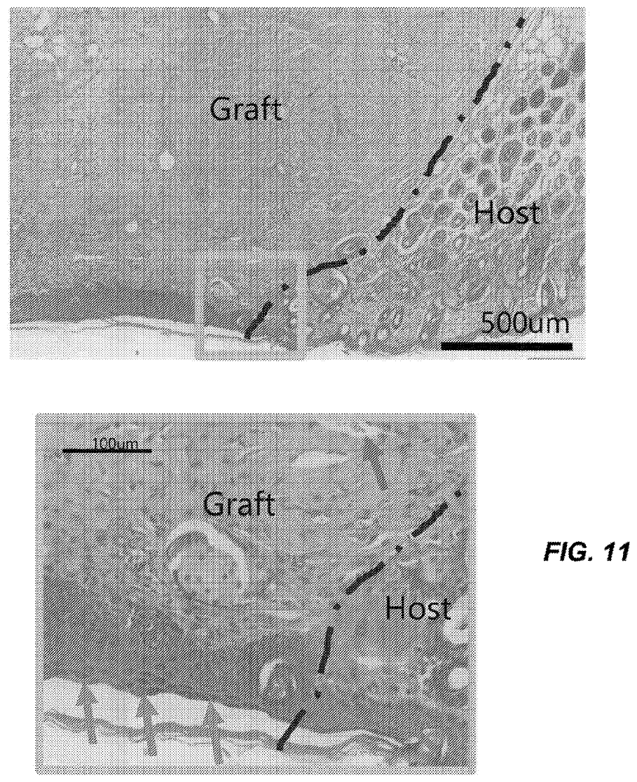

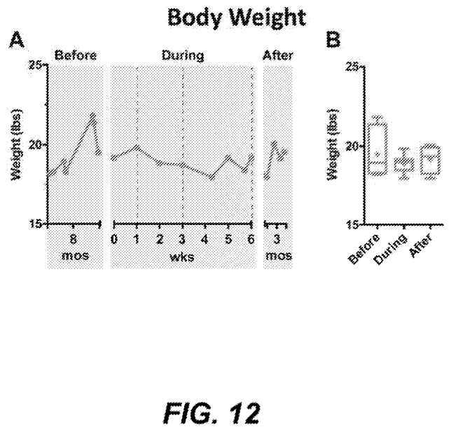

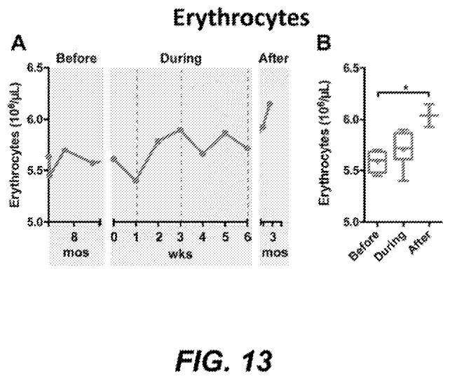

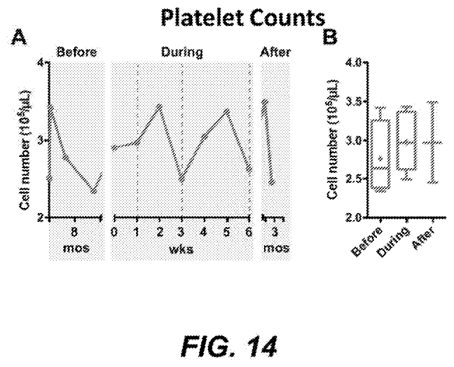

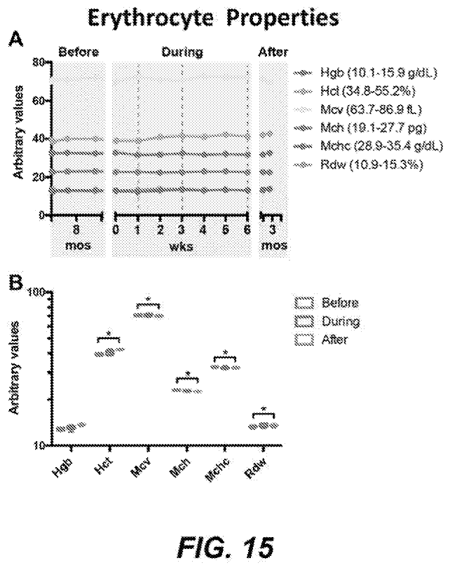

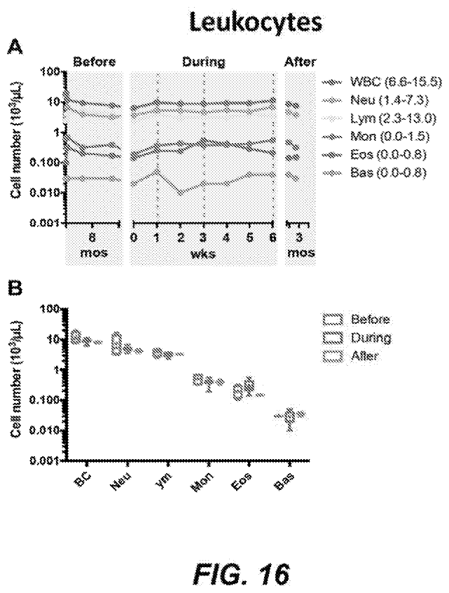

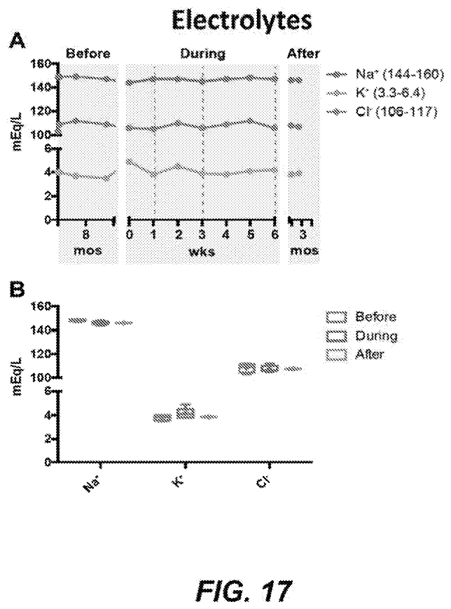

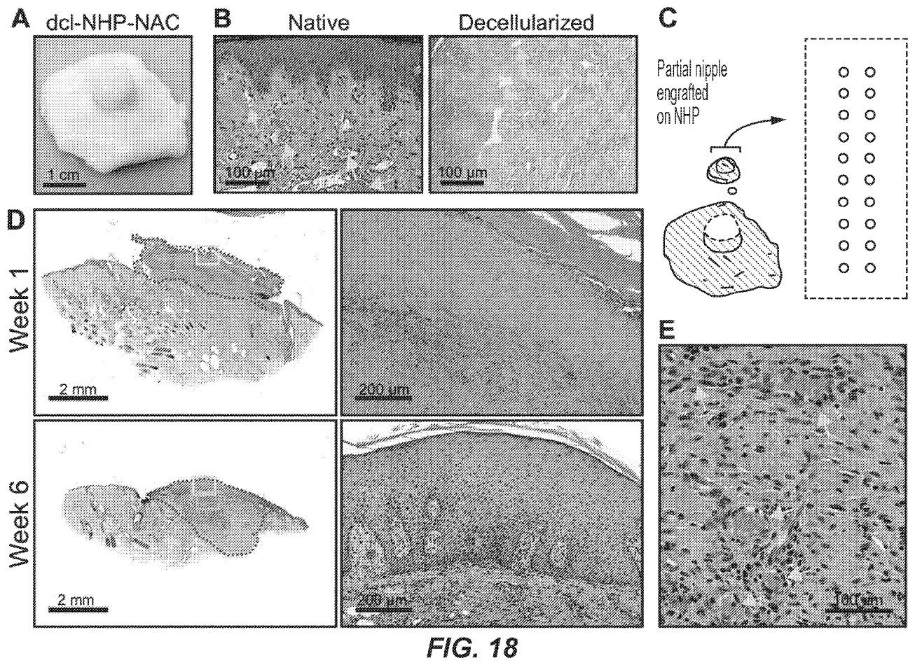

Referring to FIG. 7 to FIG. 18, the data presented here demonstrate that scaffolds are devoid of cells, retain ECM integrity, and a high-degree of bioactivity. The content of collagen and glycosaminoglycans were not significantly altered by the decellularization process; whereas, elastin content was decreased. The proliferation and apoptosis of seeded BMSCs were found to be ˜65% and <1.5%, respectively.

In vivo: Analysis of flow cytometry data, testing the blood for circulating immune cells, indicated that there was not a significant difference between the decellularized graft and commercially available decellularized dermis control. Analysis of mice weight data shows that those with decellularized NACs had a significantly higher percentage weight gain than did those with commercially available decellularized dermis. CD31+immunohistochemistry, indicated that neovascularization within the NAC grafts was present at 14 and 21 day time points, significant as compared to control (P<0.05). No local or systemic immune response was indicated as compared to the control. The feasibility study in the NHP model showed no local or systemic immune response, and re-epithelization starting within 7 days and blood vessel formation within 21 days.

These studies of the decellularized rhesus-derived nipple-areolar complex grafts show significant data that both the decellularization method identified and the biologically-derived whole nipple-areolar complex graft allow for neovascularization and minimum immune response. Together with the electron microscopy imaging of the decellularized NAC scaffold, the enrichment of structural ECM and cytoskeletal proteins demonstrated by proteomics and IHC staining suggest that the microarchitecture is sufficiently intact and the protein landscape has properties favorable for successful epithelialization and vascularization upon implantation. From these in vivo studies, it was observed that the decellularization process and the decellularized implants were able to allow for re-epithelization and blood vessel formation, and maintain a safe biocompatibility profile as compared to the controls. Without wishing to be bound by theory, these data indicate that a whole biologically-derived nipple-areolar complex is a viability and potentially helpful solution in the application of nipple-areolar complex regeneration/reconstruction after a mastectomy due to breast cancer.

### Example 4

Acellular biologically-derived grafts (ABGs) are tissues recovered from donors and then decellularized to remove cells and immunogens. For 30+ years, ABGs have been used in surgeries for abdominal hernia repair, burn wound healing, diabetic ulcer/chronic wound repair, and other indications. ABGs have relatively high complication rates that are mainly attributed to infection, seroma formation, mechanical failure, and necrosis. These complications represent a significant unmet medical need for the 7.5 million Americans treated with ABGs annually.

Embodiments described herein address this unmet medical need, and include ABGs with tunable hydrogel-based drug delivery systems for sustained, local release of therapeutic agents from the ABG itself. Impregnating ABGs with polymers for therapeutic applications has not been performed before. Incorporating hydrogel-based drug delivery into ABGs allows for low-dose, single administration of drugs at targeted sites, thus decreasing drug toxicity and its metabolic breakdown.

Using a polymer impregnation technique described herein, drug-loaded hydrogel networks within the interstitium of human dermal ABGs that readily form hydrogel networks will enable the sustained and local release of therapeutic agents from the ABG. Validation experiments can include using vancomycin—a commonly used antibiotic for the treatment of Staphylococcus aureus infection—to demonstrate sustained release from ABGs impregnated with gelatin or silk fibroin polymer hydrogel networks. For example, validation studies can use dermal ABGs impregnated with natural, biodegradable polymers mixed with vancomycin. Embodiments herein utilizes force-driven displacement of a volatile solvent from a porous material, forcing polymers and drugs in the surrounding bath solution into the dense and diffusion-limited voids of the graft. Validation studies will (1) characterize ABG impregnation with vancomycin-polymer mixtures, (2) define drug-release profile and determine drug efficacy upon release from impregnated ABGs, and (3) characterize the cellular bioactivity of the impregnated ABGs.

These studies will validate the polymer-impregnated approach and validate the use of embodiments herein as a foundation for future drug-polymer-impregnated ABG research. Without wishing to be bound by theory, endowing ABGs with additive therapeutic properties will transform tissue engineering and regenerative approaches within the clinic.

### Example 5

Specific Aims

Acellular biologically-derived grafts (ABGs) are tissues recovered from donors and decellularized to remove cells, DNA, and immunogens. Extracellular matrix (ECM) proteins, which largely comprise the architecture of most tissues, are retained during this process, resulting in a cell- and DNA-free three-dimensional protein graft. For 30+ years, ABGs have been used in surgeries for abdominal hernia repair, breast reconstruction, burn wound healing, diabetic ulcer/chronic wound repair, and other indications^{1,2}. Despite their advantages, ABGs have relatively high complication rates³⁻⁷ that are mainly attributed to infection, seroma formation, mechanical failure, and necrosis⁸⁻⁴. These complications are a significant unmet clinical need for the 7.5 million Americans treated with ABGs annually¹⁵.

To address these issues, embodiments herein comprise ABGs with tunable hydrogel-based drug delivery systems (DDS) (i.e. polymers) for sustained, local release of therapeutic agents (e.g. antibiotics, pain medications, and anti-inflammatories). Incorporating hydrogel-based drug delivery via polymers into ABGs allows for low-dose, site-specific administration of drugs, thus avoiding the toxicity risk associated with systemic administration. Impregnating ABGs with polymers for therapeutic applications has not been performed before.

Without wishing to be bound by theory, polymer-impregnated ABGs (polyABGs) loaded with drugs (drug+polyABGs) will directly address known complications of traditional ABGs and result in improved clinical outcomes. This is based upon ABGs' established natural regenerative capabilities¹⁶⁻¹⁸ for tissue reconstruction and recent therapeutic advances in hydrogel platforms for controlled drug delivery¹⁹. Using a polymer impregnation technique described herein, a human dermal drug+polyABG will be made with biocompatible, drugloadable polymers that readily form hydrogel networks to enable the sustained, local release of therapeutic agents from the graft.

Embodiments can comprise drug-loaded hydrogel networks within the interstitium of dermal ABGs. Polymer impregnation techniques described herein can utilize force-driven displacement of a volatile solvent from a porous material, forcing polymers and drugs in the surrounding bath solution into the dense and diffusion-limited voids of the ABG. Gelatin and silk fibroin, safe and natural biodegradable polymers with known drug delivery properties, will be used for the hydrogel networks. As validation studies, vancomycin will be used as the model drug. Vancomycin is a commonly used antibiotic for the treatment of post-operative Gram-positive bacterial infections such as Staphylococcus aureus (S. aureus)^{20, 21}. Without wishing to be bound by theory, sustained release of vancomycin from ABGs impregnated with different biodegradable polymers will be shown.

The Aims of the validation studies comprise:

Aim 1. Characterize ABG Impregnation with Vancomycin Polymer Mixtures.

ABGs will be force-impregnated with vancomycin-polymer mixtures, testing gelatin and silk fibroin polymers at two concentrations. These vancomycin-polymer-impregnated ABGs (vanc+polyABGs) will be characterized for polymer occupancy, which measures the efficiency of polymer impregnation and is important for drug delivery characteristics. Tensile testing will be performed to characterize the mechanical properties of the vanc+polyABGs, since altered mechanical properties may affect ABG performance. Such studies will determine impregnation conditions that produce a polymer occupancy≥5% of the vanc+polyABG volume and a polymer distribution such that normalized polymer occupancy is ±40% of the mean. Further, such studies will characterize the Young's modulus, tensile strength, and Poisson's ratio of vanc+polyABGs.

Aim 2. Define Drug-Release Profile and Determine Drug Efficacy Upon Release from Impregnated ABGs.

The kinetics (burst release parameter, half-life) of vancomycin release from vanc+polyABGs will be measured through HPLC-based detection. To measure efficacy of vancomycin upon release from vanc+polyABGs, Kirby-Bauer disk diffusion susceptibility testing against vancomycin-sensitive S. aureus will be used. Such studies will allow for observation of drug release, at or exceeding the minimum inhibitory concentration for S. aureus (1 μg/mL), after 20 days. Further, such studies will measure the effect on drug release kinetics from different hydrogel types (gelatin, silk) and concentrations.

Aim 3. Characterize the Cellular Bioactivity of Impregnated ABGs.

The bioactivity of vanc+polyABGs will be determined by seeding them with primary human dermal fibroblasts and measuring cell viability, proliferation, and apoptosis. Such studies will allow for the validation of impregnated ABGs' biocompatibility by showing cell populations that are ≥80% viable, ≥50% proliferative, and <1% apoptotic.

Together, these studies will validate the polymer-impregnated approach. Ultimately, the goal is improving clinical outcomes by reducing complications that arise from ABG surgery-related infections. Endowing ABGs with therapeutic properties will transform the fields of reconstructive surgery and wound healing.

Significance

Biologically-derived and synthetic grafts are used in a wide variety of surgical procedures. Biologically-derived grafts are materials (skin, tendons, bone grafts, etc.) taken from donors. These biologically derived grafts can be autografts, allografts, or xenografts and can be cellular (CBGs) or acellular (ABGs). CBGs retain the donor's native cells and DNA and are usually sterilized to minimize infection risk. CBGs are very similar in structure and function to the host tissues that they are replacing, but the CBGs suffer from serious limitations, a consequence of containing cells, such as the requirement of donor/recipient matching and extensive immunesuppression to prevent rejection as well as immediate, adequate blood supply to prevent infection and necrosis.

Acellular biologically-derived grafts (ABGs) were created to address CBG rejection, infection, and necrosis issues^{7, 22, 23}. Decellularization methods remove the donor cells and DNA from CBGs, leaving only the protein rich native extracellular matrix (ECM). ABGs meet well-established criteria for DNA concentration^{24, 25} and fragment size that have been shown to be non-immunogenic and, therefore, eliminates the risk of immune rejection, need for donor/recipient matching, and treatment with anti-rejection immune-suppressing medication. Since the ABGs are devoid of cells, they do not require an immediate blood supply to prevent necrosis.

ABGs are routinely used for hernia repair, breast reconstruction, and wound management (burns, diabetic ulcers)^{1, 2, 13-15}. An estimated 350,000 ventral hernia repairs are performed annually in the US with either synthetic grafts or ABGs^{26, 27}. Because ABGs are associated with lower rates of infection and, due to faster healing rates, are less likely to require removal than synthetic grafts, ABGs are more commonly used in hernia repair²⁷⁻³². Furthermore, synthetic grafts have been heavily litigated, particularly for uses in ventral and pelvic floor hernia repair³³⁻³⁹. Breast reconstruction accounts for significant clinical usage of ABGs; approximately 75,000 women have breast reconstructions utilizing ABGs annually⁴⁰⁻⁴². ABGs are also widely used in wound management and repair. Chronic wounds, which include vascular ulcers (venous and arterial ulcers), diabetic ulcers, and pressure ulcers, affect an estimated 4.5 million people in the US43. ABGs significantly increase the probability of successful wound healing in diabetic foot ulcers⁴⁴ and severe burns^{45, 46} over standard of care. Mean healing times for serious diabetic foot ulcers are halved with ABGs (40 days) as compared to conventional care (77 days)^{47, 48}.

Despite their numerous advantages and widespread usage, meta-analyses on large clinical studies for ABGs in hernia repair and breast reconstruction show ABGs have relatively high complication rates of 10-23%³⁻⁷, which are mainly attributed to infection (cellulitis), seroma formation, mechanical failure, and necrosis⁸⁻¹⁴. Closer analyses into ABG failures show reduced vascularization, poor mechanical integrity, failure to integrate (resulting in scarring), and immune rejection^{18, 49-53}. Further, infection is the single most common post-operative complication with ABGs⁵⁴. Surgical sites are highly susceptible to colonization by pathogenic bacteria such as Staphylococcus aureus (S. aureus); therefore, the use of local and systemic antibiotic administration is frequently prescribed to post-operative patients. However, these antibiotics are not fully effective due to limitations of the delivery methods (e.g. drug metabolism, inefficient delivery to relevant site, etc.). Abdominal hernia repairs have shown 25% infection rates that are nearly as high as the 27% hernia recurrence rate⁵⁵. Clinical studies using ABGs for breast reconstruction have shown a reduced infection rate compared to hernia repairs, but a rate that is still prevalent at 6.9%⁴¹. These complications result in graft failure and require repeat surgery, increasing the risk of further complications to the patient and increasing cost. Improved solutions are required to combat this significant unmet medical need.

The ideal graft is biocompatible and resistant to infection, thrombosis, necrosis, and mechanical failure. Embodiments herein comprise a dermal ABG impregnated with biodegradable, biocompatible, drug-loaded polymers (drug+polyABG) that readily form hydrogel networks to enable the sustained and local release of therapeutic agents from the drug+polyABG. Without wishing to be bound by theory, drug+polyABGs delivering antibiotics, analgesics, and/or anti-inflammatory agents will address issues with infection, pain, and host hyperinflammatory responses, respectively, thus increasing graft acceptance and host integration, lessening the likelihood of complications, and improving patient pain management, healing, and patient recovery time. Additionally, the impregnated polymers may enhance the drug+polyABGs' mechanical properties, namely elasticity and tensile strength.

Embodiments described herein will address the significant unmet medical need of ABG complications to improve clinical outcomes for the 7.5 million Americans treated with ABGs each year¹⁵.

Rigor of the Prior Research:

ABGs can be impregnated with drug-loaded polymers for therapeutic applications, a novel concept that has not been performed previously. ABGs are FDA approved and have been used in reconstructive surgeries for more than 30 years^{26-32, 41-47, 56}. Validation studies will use gelatin and silk fibroin polymers. Gelatin and silk fibroin have been extensively used in biomedical applications, particularly for hydrogel based drug delivery⁵⁷⁻⁶⁵. Both polymers are generally regarded as safe (GRAS) by the FDA, non-immunogenic, biocompatible, and promote cell adhesion and proliferation66; they possess relatively small and simple monomeric forms, are biologically-derived, and readily crosslink through chemical (pH, alcohol) and physical (temperature, sonication) mechanisms. Silk fibroin is also well known for its mechanical resilience^{61, 67, 68}.

Innovation:

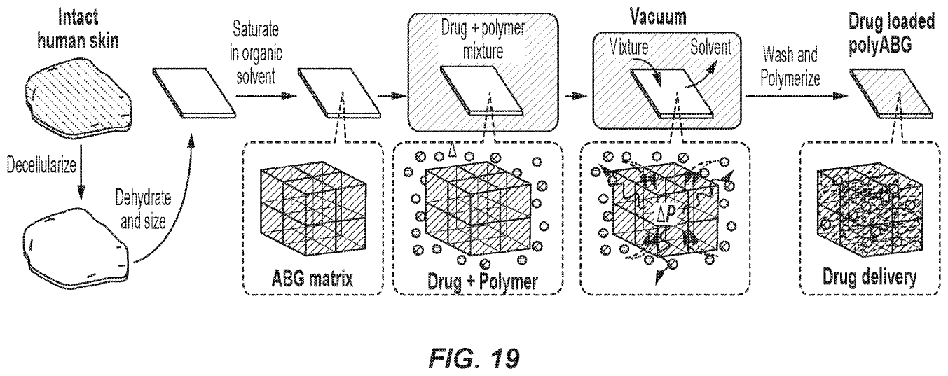

Embodiments herein comprise an impregnation technique that is based upon force-driven displacement of volatile solvents, forcing polymers and drugs from the surrounding bath solution into the dense, diffusion-limited voids of the ABGs to create drug+polyABGs (FIG. 19). The principles of this nondestructive technique are similar to that used in industrial processes for strengthening porous materials and have been used in tissue and whole cadaver preservation for teaching purposes⁶⁹⁻⁷². This approach is novel, as impregnating ABGs with polymers for therapeutic applications has not been performed before.

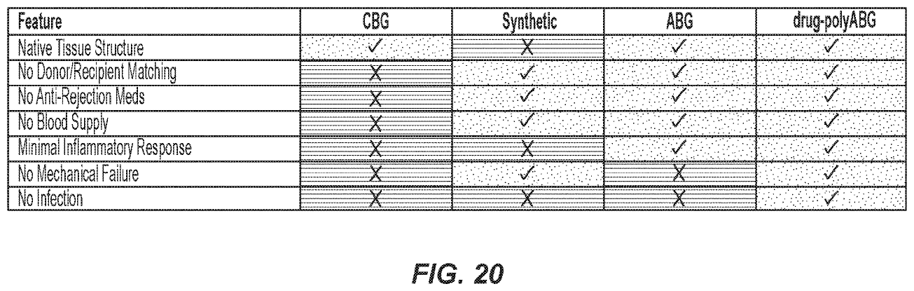

Polymer embedded grafts have been described previously⁷³; however, these are often hydrogel networks and 3D-printed scaffolds comprised of single polymer mixtures, as opposed to naturally occurring ABGs⁷³. Without wishing to be bound by theory, ABGs have not been previously used for hydrogel-based drug delivery systems due to the diffusion limited nature of the ECM. Our innovative approach overcomes this issue and drives polymers into the dense, native ECM of the ABG, allowing polymers to permeate the tissue interstitium (see data herein). Unlike polymer embedded grafts which attempt to recreate ECM, our polyABG approach utilizes actual ECM, harnessing its natural morphological complexity and cell amenable properties. FIG. 20 demonstrates the strengths of our drug+polyABG versus current options.

Framework:

The key steps involved in creating drug+polyABGs comprise 1) decellularization of donor tissue, 2) force-driven impregnation of hydrogel-forming natural polymers, and 3) hydrogel-based drug release from the drug+polyABGs.

1) Decellularization:

Decellularization approaches are each optimized for specific tissue and greatly preserve the composition, structure and mechanical complexity of the ECM. Decellularization techniques for whole organ regeneration of lung in rat and non-human primate (NHP) models have previously been optimized^{74, 75}, and have successfully employed these approaches on human skin to create human dermal ABGs²⁴. The decellularization process meets widely-accepted criteria for generating non-immunogenic acellular tissue post decellularization^{24, 25}. For example, the decellularization method can comprise that described in US 2018/0015204, which is incorporated herein by reference in its entirety. The decellularization process on NHP dermis for the retention of ECM components collagen, glycosaminoglycans (GAGs), laminin, fibronectin, and elastin has been characterized²⁴. The preservation of these molecules to levels similar to intact dermis has also been confirmed. The levels of retained ECM components from decellularized NHP dermis are similar to data from other epithelial tissue such as lung from rhesus macaque and rat, and skin from pig⁷⁷⁻⁸⁰. Furthermore, the decellularized NHP dermis supports cell proliferation (>65%), cell migration, and does not significantly induce apoptosis (<1.5%)²⁴. Lastly, in vivo experiments on NHPs with our decellularized human dermal ABGs have been completed. These ABGs support reepithelialization and neovascularization, showing near complete epithelial coverage at 6 weeks post-engraftment (91.4±8.6% coverage, n=6 ABGs). Neovascular formation, as measured by PECAM1+ vessel lumens, occurred within these ABGs as early as 1-week post engraftment, with more mature vessel formation, as well as increased presence of vessels, occurring over time. These data demonstrate the decellularization process is appropriately optimized for skin, creating a dermal ABG that supports regeneration.

2) Polymer Impregnation:

The polymer impregnation concept herein can comprise those of industrial processes for fortifying porous materials such as wood, and plastination for preserving tissues and whole organisms⁶⁹⁻⁷². Both applications similarly involve replacement of water from samples with organic solvents possessing high vapor pressures. Upon vacuum application, the solvent within the sample becomes volatile and undergoes a phase transition from liquid to gas. This solvent transition and resultant evacuation from the sample creates a negative pressure within the sample that forces bath solutions, such as polymers and drugs, into the material. This technique has been used in industrial applications with a range of substances of various molecular sizes and charges⁸¹⁻⁸⁵.

The approach and application herein differs from prior polymer impregnation uses in several key areas. First, the application is for generation of a hydrogel-based drug delivery system for therapeutic purposes. This vastly differs from prior uses of polymer impregnation which have exclusively been used for material fortification and preservation. Second, embodiments herein are impregnating biomaterials with biocompatible, natural polymers. This greatly differs from prior uses of polymer impregnation in which polymers such as adhesives, resins, and alloys are used. Lastly, the sample material is an ABG, not a fixed cadaveric tissue or man-made material. Because the sample materials differ, the conditions for polymer impregnation do as well. Polymer impregnation conditions have been extensively optimized to be suitable for dermal ABGs. Impregnation conditions established for fixed, intact tissue do not result in impregnation of acellular tissue.

3) Hydrogel-Based Drug Delivery:

Natural biodegradable polymers have long been used in drug delivery systems (DDS) as carriers⁸⁶. They can form hydrogel networks that enable controlled release of hydrophobic and hydrophilic drugs in constant doses over long periods⁸⁶. Their biodegradable properties allow for gradual disappearance, eliminating removal of the drug carrier itself. Natural hydrogel DDS are fully tunable, allowing for control of their network pore size. This is important since the pores of the hydrogel will entrap the drug and, depending on the nature of the drug, pore size can be adjusted to physically entrap small versus large therapeutic agents. Gelatin and silk fibroin, for example, can be utilized in validation studies since they are well-established natural polymers that are amenable to drug loading and release⁵⁷⁻⁶⁵. Knowledge and methods of drug delivery and hydrogel-based drug delivery will be utilized to enable sustained drug release from an ABG over time. Biodegradable polymers used to encapsulate and release growth factors in a controlled, sustained manner has previously been shown⁸⁷. This study used a novel alginate construct as a multi-functional tissue scaffold for central nervous system repair that delivered a brain-derived neurotrophic factor (Neurotrophin-3)⁸⁷. In our drug+polyABG program, we will use similar methods to physically entrap vancomycin in gelatin and silk fibroin hydrogels.

Localized drug delivery is better suited for surgical site infections as it provides a higher therapeutic dose at the relevant site of drug action, avoids drug metabolism, and reduces off-target effects. A hydrogel-based ABG DDS will provide sustained, local release, allowing for low-dose, targeted, non-systemic administration for surgical site infections.

Preliminary Data:

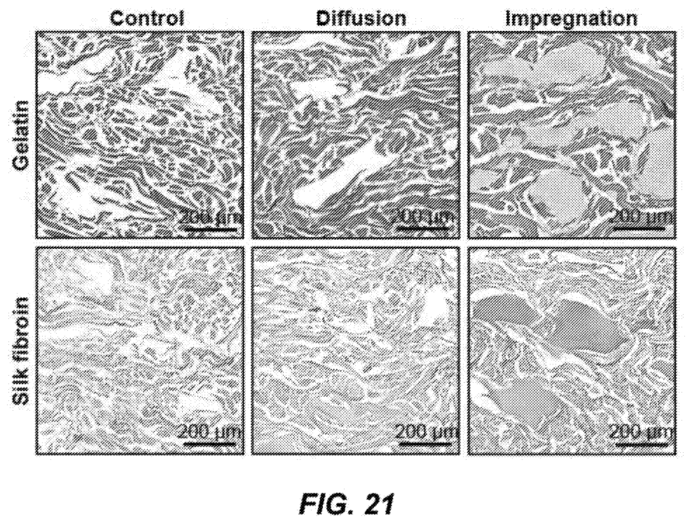

Several natural polymers have been shown to function as DDS^{57-61, 86-94} Validation studies have focused on gelatin and silk fibroin since both have been extensively studied for use in drug delivery⁵⁷⁻⁶⁵. Through the polymer impregnation approach described herein, hydrogel networks within ABGs have been generated. Studies have identified impregnation conditions that lead to successful polymer impregnation within dermal ABGs, enabling slight to near complete impregnation of interstitial spaces. These studies were largely performed with natural polymers and, to a lesser extent, synthetic polymers. Preliminary data from both natural and synthetic polymers consistently demonstrate that polymer impregnation via force-driven displacement of a volatile solution from the ABG vastly outperforms diffusion alone. Gravity-based diffusion of polymeric solutions will penetrate hundreds of microns into the graft, exclusively occupying the perimeter of the graft whereas force-driven impregnation will lead to polymer occupancy throughout the entire graft, with penetration depths in the several of millimeters (FIG. 21). These data point to the natural diffusion-limited properties of the dermal ABG and how our polymer-impregnation approach overcomes this limitation. These validation studies demonstrate the ability to generate gelatin and silk fibroin hydrogels within ABGs.

Approach:

Gelatin and silk fibroin polymers will be used to physically entrap vancomycin within a dermal ABG. Vancomycin was chosen as the model drug as it is highly effective against pathogenic Gram-positive bacteria that are common to surgical infections, such as S. aureus, Clostridium difficile, and Staphylococcus epidermidis^{20, 21}. Vancomycin remains a drug of choice for treatment of severe methicillin resistant S. aureus infections²⁰. Although S. aureus strains with complete resistance to vancomycin (minimum inhibitory concentration=16 μg/mL) do exist, these are relatively rare (14 total cases in U.S.)²⁰

Aim 1. Characterize ABG Impregnation with Vancomycin Polymer Mixtures.

This Aim addresses the ability to impregnate ABGs with gelatin and silk fibroin polymer/vancomycin mixtures. Different polymer concentrations will be tested as this property greatly controls hydrogel pore size¹⁹, hydrogel pore size controls the diffusivity of physically entrapped agents, specifically release concentration and diffusion length. Thus, vancomycin release kinetics are expected to differ with polymer concentration, showing greater burst release and shorter release length with lower concentration versus higher concentration polymer. Because polymers that possess measurable mechanical properties are being used, particularly in the case of silk fibrin, tensile properties will be measured to determine how these polymers affect ABG mechanical properties.

Validation Approach:

Generation of vanc+polyABGs.

An illustration of this approach is shown in FIG. 19. Briefly, donor derived human skin is decellularized using our patent-pending process and cut to 1×3 cm rectangles at 0.4 cm thickness, and then dehydrated. Separate polymer solutions of gelatin and silk fibroin with 7.5 mg/mL vancomycin (concentration is 10-fold greater than the minimum inhibitory concentration for S. aureus and within non-toxic range for blood serum levels95) will be prepared. Two concentrations of each polymer will be tested: 5% and 20% w/v for gelatin and 1% and 5% w/v for silk fibroin. The dehydrated ABG will be saturated in an organic solvent and incubated with polymer and drug mixtures and then force-impregnated using process described herein. The vanc+polyABG will be washed and set under conditions for polymerization for gelatin and silk fibroin (gelatin: 4° C., methanol; silk fibroin: 37° C., ethanol). These conditions do not adversely affect vancomycin solubility or activity⁹⁶. Controls will include ABG alone and polyABG without vancomycin. A total of 27 samples (2 polymers, 2 concentrations, 2 controls/condition, ABG alone control, n=3) will be analyzed. Samples will be analyzed via histology with hematoxylin and eosin (H&E) [gelatin] and alcian blue [silk fibroin].

Measure Polymer Occupancy and Distribution within Vanc+polyABG.

Polymer occupancy will be quantified—polymer signal area (measured by histological stains)/total ABG area. This measure shows the efficiency of polymer impregnation and is important for drug delivery characteristics. Image analysis with channel thresholding and segmentation analyses tools (ImageJ) will be used. Distribution of normalized polymer occupancy will be analyzed−% area polymer/% area void prior to impregnation—throughout the ABGs for deviation from the mean. Normalizing is used because, without wishing to be bound by theory, the amount of polymer entering a region of the ABG is directly related to the amount of void space that existed prior to impregnation. Statistical analyses between polymer impregnation conditions and condition interactions will be performed with a two-way ANOVA (α=0.05).

Measure Mechanical Properties of Vanc+polyABGs.

Uniaxial tensile testing on a micro-strain analyzer (TSA Instruments) according to ASTM D63897 will be performed. Stress:strain profiles for ABGs will be generated and the Young's modulus, tensile strength, and Poisson's ratio will be characterized. A total of n=15 replicates/ABG condition will be analyzed. Controls will include ABG alone and polyABG without vancomycin. A total of 135 samples (2 polymers, 2 concentrations, 2 controls/condition, ABG alone control, n=15) will be analyzed. Differences in mechanical property measures will be analyzed with one-way ANOVA (α=0.05).

Results:

Without wishing to be bound by theory, polymer occupancy≥5% vanc+polyABG area and polymer distribution such that normalized polymer occupancy is ±40% of the mean. Because skin is stratified, ECM density is not uniform across its thickness. Thus, hydrogel distribution variability across the thickness of the vanc+polyABGs may occur. Anticipated values are based on Monte Carlo simulations. Polymer impregnation should affect the overall mechanical properties of the ABG; the extent of this influence is unknown. At these minimum polymer occupancy and distribution values, vanc+polyABGs containing silk fibroin—but not those containing gelatin—should have significantly altered mechanical properties, specifically showing increases in Young's modulus, tensile strength, and Poisson's ratio. The ideal vanc+polyABG will have a Young's modulus and tensile strength greater than that of traditional, commercially available dermal ABGs^{87, 98-102}.

Aim 2. Define Drug-Release Profile and Determine Drug Efficacy Upon Release from Impregnated ABGs.

This Aim validates whether vanc+polyABGs will release vancomycin in a manner that impacts S. aureus survival. This Aim will validate drug release and activity from vanc+polyABGs. Measuring drug release kinetics is essential to determine if therapeutic potential can be expected, based on drug release concentration, duration, and profile (i.e. continuous versus pulsatile). These profiles will show if drug release properties differ with polymer type and concentration. Drug efficacy after release will be measured against vancomycin-sensitive S. aureus to demonstrate potential therapeutic effect. S. aureus was chosen as it is common in surgical infections. Vancomycin is an effective treatment for S. aureus. Since drug release alone does not imply drug activity, particularly over time, this assay provides a functional output for drug activity and a quantitative approach to measuring S. aureus susceptibility. Aim 2 will validate vanc+polyABGs generated under conditions identified in Aim 1 that meet the identified polymer occupancy and distribution thresholds.

Validation Approach:

Drug release kinetics (burst release parameter, half-life) of vanc+polyABG (generated via same approach in Aim 1) will be measured using a 6-sample Vertical Diffusion Cell (Teledyne Hanson Research) and HPLC-based detection¹⁰³. The Vertical Diffusion Cell is commonly used for percutaneous absorption studies of dermal and transdermal drug delivery systems¹⁰⁴. ABG samples will be placed against a porous membrane in a physiological saline bath. Released drug will pass through the membrane to receptor medium over time. Samples will be collected at 0, 1, 3, 10, 100 (4 d), 300 (13 d), and 500 hours (21 d) for each ABG. Collected drug release samples will be analyzed via HPLC103 (Agilent 1220). HPLC is the most sensitive and common method for vancomycin identification and purity determination¹⁰⁵⁻¹⁰⁷. Controls will include ABG alone, polyABG without vancomycin, and vancomycin saturated ABG (no polymer). A total of 210 samples (2 polymers, 2 concentrations, 2 controls/condition, ABG+vanc (no polymer), ABG alone, 7 timepoints, n=3) will be run for the drug kinetics work. Descriptive statistics will be compiled for each vanc+polyABG. To measure vancomycin efficacy, the Kirby-Bauer disk diffusion susceptibility test will be used according to standard technique with S. aureus (ATCC 29213)¹⁰⁸. Drug efficacy is measured as zone of inhibition (ZOI)—area in which challenge organism does not grow. In this assay, receptor medium samples from the Vertical Diffusion Cell will be blotted on wafers and placed on confluent S. aureus culture plates for 18 hrs. Receptor medium samples collected at 0, 1, 3, 10, 100 (4 d), 300 (13 d), and 500 hours (21 d) for each ABG will be tested. Controls include ABG alone and polyABG without vancomycin. A total of 252 samples (2 polymers, 2 concentrations, 2 controls/condition, 7 timepoints, n=3) will be run. Additionally, a 75 μg/mL vancomycin-loaded wafer will be a positive control. The ZOI will be photographed and measured using ImageJ for each time point. If antibiotic resistance occurs, the data may be non-Gaussian. Studies will use a non-parametric Kruskal-Wallis one-way ANOVA by ranks (α=0.05) to analyze these data, since this test makes no assumptions about the normality of the data.

Results:

Without wishing to be bound by theory, vancomycin released from the vanc+polyABGs will be detected after 20 days, at effective concentrations at or exceeding lethal levels for S. aureus. If so, no S. aureus growth is expected. The ZOI will decrease over time, as less vancomycin is released from vanc+polyABGs. The assay should have a lower ZOI quantitation limit of 50 μg/mL vancomycin¹⁰⁹. Given differences in polymer hydrogel degradation rates, gelatin should release vancomycin faster than silk fibroin.

Aim 3. Characterize the Cellular Bioactivity of Impregnated ABGs.

This Aim validates that a vanc+polyABG is bioactive, supports cell viability and proliferation, and will not induce apoptosis. All polymer concentrations from Aim 1 will be tested in Aim 3. The bioactivity of vanc+polyABGs will be determined by measuring cell activities in normal adult human primary dermal fibroblasts (HDFa) cultured in the presence of vanc+polyABGs. HFDa cells are adherent cells originally isolated from adult dermis, similar to our ABGs, and require cell-substrate interaction for survival. Cell viability, proliferation, and apoptosis are robust, commonly used metrics to assess cytoxic effects and are thus appropriate to fulfill this Aim. These experiments will provide measurable evidence for the presence or absence of cytotoxicity due to vancomycin release (concentration and duration) and/or the presence of polyABGs themselves.

Validation Approach: Load vanc+polyABGs (1 cm diameter circles) into 48-well plates. Controls will include ABG alone, polyABG without vancomycin, and vancomycin saturated ABG (no polymer). A total of 100 samples (2 polymers, 2 concentrations, 2 controls/condition, ABG+vanc (no polymer), ABG alone, 2 timepoints, n=5) will be analyzed. HDFa cells (ATCC, PCS-201-012) will be seeded onto ABGs (5×104 cells/ABG) and cultured for 1 and 2 weeks. Cell viability will be measured via calcein-AM and ethidium homodimer-1 staining (LIVE/DEAD assay, Thermo). Cell proliferation will be measured via Ki-67 immuno-staining (anti-Ki67, Cell Signaling) and EdU incorporation (Click-iT EdU assay, Invitrogen). Apoptosis will be measured with the TUNEL assay (In Situ Cell Death Detection Kit, Roche) with kit-appropriate positive and negative controls24, 77. All samples for each assay will be imaged on a wide-field fluorescent microscope as each output is fluorescent. Measurements will be taken from 5 random fields of view/ABG for each assay and background subtracted. ImageJ software will be used for image quantification. Statistical analysis will be One-way ANOVA (α=0.05) with Tukey's posthoc test.

Results:

Similar assays were performed with rhesus macaque bone marrow-derived mesenchymal stem cells²⁴, and expect similar results in this Aim: ≥80% cell viability, ≥50% proliferative cells, and ≤1% apoptotic cells.

PolyABG Development.

A novel drug+polyABG can address clinically available grafts limitations: infections, scarring, poor mechanical integrity, and poor host integration. Subsequent studies can involve other clinically relevant infections, including methicillin-resistant S. aureus (MRSA).

### REFERENCES CITED IN THIS EXAMPLE

1. Jeffcoate W J, Vileikyte L, Boyko E J, Armstrong D G, Boulton A J M. Current Challenges and Opportunities in the Prevention and Management of Diabetic Foot Ulcers. Diabetes Care. 2018; 41(4):645-52.
2. Scarritt M E, Pashos N C, Bunnell B A. A review of cellularization strategies for tissue engineering of whole organs. Front Bioeng Biotechnol. 2015; 3:43.
3. Kim J Y, Davila A A, Persing S, Connor C M, Jovanovic B, Khan S A, Fine N, Rawlani V. A meta-analysisof human acellular dermis and submuscular tissue expander breast reconstruction. Plast ReconstrSurg. 2012; 129(1):28-41.
4. Ho G, Nguyen T J, Shahabi A, Hwang B H, Chan L S, Wong A K. A systematic review and meta-analysisof complications associated with acellular dermal matrix-assisted breast reconstruction. Ann Plast Surg.2012; 68(4):346-56.
5. Loo Y L, Kamalathevan P, Ooi P S, Mosahebi A. Comparing the Outcome of Different BiologicallyD erived Acellular Dermal Matrices in Implant-based Immediate Breast Reconstruction: A Meta-analysis of the Literatures. Plast Reconstr Surg Glob Open. 2018; 6(3):e1701.
6. Trippoli S, Caccese E, Tulli G, Ipponi P, Marinai C, Messori A. Biological meshes for abdominal hernia: Lack of evidence-based recommendations for clinical use. Int J Surg. 2018; 52:278-84.
7. Slater N J, van der Kolk M, Hendriks T, van Goor H, Bleichrodt R P. Biologic grafts for ventral herniarepair: a systematic review. Am J Surg. 2013; 205(2):220-30.
8. Weichman K E, Wilson S C, Weinstein A L, Hazen A, Levine J P, Choi M, Karp N S. The use of acellulardermal matrix in immediate two-stage tissue expander breast reconstruction. Plast Reconstr Surg.2012; 129(5):1049-58.
9. Chun Y S, Verma K, Rosen H, Lipsitz S, Morris D, Kenney P, Eriksson E. Implant-based breastreconstruction using acellular dermal matrix and the risk of postoperative complications. Plast ReconstrSurg. 2010; 125(2):429-36.
10. Woodward J, Wright A. Aspergillus infection of abdominal wall biologic mesh. Surg Infect (Larchmt).2010; 11(4):405-6.
11. Harth K C, Rosen M J. Major complications associated with xenograft biologic mesh implantation inabdominal wall reconstruction. Surg Innov. 2009; 16(4):324-9.
12. Awad S S, Rao R K, Berger D H, Albo D, Bellows C F. Microbiology of infected acellular dermal matrix(AlloDerm) in patients requiring complex abdominal closure after emergency surgery. Surg Infect(Larchmt). 2009; 10(1):79-84.
13. Shah B C, Tiwari M M, Goede M R, Eichler M J, Hollins R R, McBride C L, Thompson J S, Oleynikov D. Notall biologics are equal! Hernia. 2011; 15(2):165-71.
14. Chavarriaga L F, Lin E, Losken A, Cook M W, Jeansonne L O, White B C, Sweeney J F, Galloway J R, Davis S S, Jr. Management of complex abdominal wall defects using acellular porcine dermal collagen. Am Surg. 2010; 76(1):96-100.
15. Vacanti J P, Langer R. Tissue engineering: the design and fabrication of living replacement devices forsurgical reconstruction and transplantation. Lancet. 1999; 354 Suppl 1:S132-4.
16. He C, Yang Z, Jin Y, Qi X, Chu J, Deng X. ADM Scaffolds Generate a Pro-regenerativeMicroenvironment During Full-Thickness Cutaneous Wound Healing Through M2 MacrophagePolarization via Lamtor1. Front Physiol. 2018; 9:657.
17. Yu Y, Alkhawaji A, Ding Y, Mei J. Decellularized scaffolds in regenerative medicine. Oncotarget.2016; 7(36):58671-83. doi: 10.18632/oncotarget.10945.
18. Vig K, Chaudhari A, Tripathi S, Dixit S, Sahu R, Pillai S, Dennis V A, Singh S R. Advances in SkinRegeneration Using Tissue Engineering. Int J Mol Sci. 2017; 18(4).
19. Li J, Mooney D J. Designing hydrogels for controlled drug delivery. Nature Reviews Materials. 2016; 1:16071.
20. McGuinness W A, Malachowa N, DeLeo F R. Vancomycin Resistance in Staphylococcus aureus. Yale J Biol Med. 2017; 90(2):269-81.
21. Levine D P. Vancomycin: a history. Clin Infect Dis. 2006; 42 Suppl 1:S5-12.
22. Liang R, Knight K, Easley D, Palcsey S, Abramowitch S, Moalli P A. Towards rebuilding vaginal supportutilizing an extracellular matrix bioscaffold. Acta Biomater. 2017; 57:324-33.
23. Fosnot J, Kovach S J, 3rd, Serletti J M. Acellular dermal matrix: general principles for the plasticsurgeon. Aesthet Surg J. 2011; 31(7 Suppl):5S-12S.
24. Pashos N C, Scarritt M E, Eagle Z R, Gimble J M, Chaffin A E, Bunnell B A. Characterization of anAcellular Scaffold for a Tissue Engineering Approach to the Nipple-Areolar Complex Reconstruction. Cells Tissues Organs. 2017; 203(3):183-93.
25. Crapo P M, Gilbert T W, Badylak S F. An overview of tissue and whole organ decellularization processes. Biomaterials. 2011; 32(12):3233-43.
26. Poulose B K, Shelton J, Phillips S, Moore D, Nealon W, Penson D, Beck W, Holzman M D. Epidemiology and cost of ventral hernia repair: making the case for hernia research. Hernia. 2012; 16(2):179-83.
27. Harris H W. Clinical outcomes of biologic mesh: where do we stand? Surg Clin North Am.2013; 93(5):1217-25.
28. Diaz J J, Jr., Guy J, Berkes M B, Guillamondegui O, Miller R S. Acellular dermal allograft for ventralhernia repair in the compromised surgical field. Am Surg. 2006; 72(12):1181-7; discussion 7-8.
29. Sandvall B K, Suver D W, Said H K, Mathes D W, Neligan P C, Dellinger E P, Louie O. Comparison ofSynthetic and Biologic Mesh in Ventral Hernia Repair Using Components Separation Technique. Ann Plast Surg. 2016; 76(6):674-9
30. Darehzereshki A, Goldfarb M, Zehetner J, Moazzez A, Lipham J C, Mason R J, Katkhouda N. Biologicversus nonbiologic mesh in ventral hernia repair: a systematic review and meta-analysis. World J Surg.2014; 38(1):40-50.
31. Ventral Hernia Working G, Breuing K, Butler C E, Ferzoco S, Franz M, Hultman C S, Kilbridge J F, Rosen M, Silverman R P, Vargo D. Incisional ventral hernias: review of the literature and recommendation sregarding the grading and technique of repair. Surgery. 2010; 148(3):544-58.
32. Kockerling F, Alam N N, Antoniou S A, Daniels I R, Famiglietti F, Fortelny R H, Heiss M M, Kallinowski F, Kyle-Leinhase I, Mayer F, Miserez M, Montgomery A, Morales-Conde S, Muysoms F, Narang S K, Petter-Puchner A, Reinpold W, Scheuerlein H, Smietanski M, Stechemesser B, Strey C, Woeste G, Smart N J. What is the evidence for the use of biologic or biosynthetic meshes in abdominal wallreconstruction? Hernia. 2018; 22(2):249-69.
33. Pelley S. Gynecological Mesh: The Medical Device that has 100,000 Women Suing 2018. Available from: https://www.cbsnews.com/news/boston-scientific-gynecological-mesh-the-medical-device-thathas-100000-women-suing/.
34. BBC News. Vaginal mesh implants: Australia apologises for ‘decades of pain’ 2018. Available from: https://www.bbc.com/news/world-australia-45806324.
35. Campbell P, Jha S, Cutner A. Vaginal mesh in prolapse surgery. The Obstetrician & Gynaecologist.2018; 20:49-56.
36. Scott S, Branley A. Mesh implants: Government issues national apology over ‘agony and pain’ causedby device: ABC News; 2018. Available from: https://www.abc.net.au/news/2018-10-10/mesh-implantsgovernment-issues-apology-to-women/10355546?section=health.
37. Australia Co. Number of women in Australia who have had transvaginal mesh implants and relatedmatters. 2018.
38. Perriello B. Johnson & Johnson's Ethicon loses $35 m pelvic mesh lawsuit 2018. Available from: https://www.massdevice.com/johnson-johnsons-ethicon-loses-35m-pelvic-mesh-lawsuit/.
39. York M A. Ethicon, Inc. and J&J facing thousands of TVM and hernia mesh lawsuits: will they settlesooner or later? 2018. Available from: https://www.masstortnexus.com/mass-torts-news/ethicon-incand-jj-facing-thousands-of-tvm-and-hernia-mesh-lawsuits-will-they-settle-sooner-or-later/.
40. Jagsi R, Jiang J, Momoh A O, Alderman A, Giordano S H, Buchholz T A, Kronowitz S J, Smith B D. Trends and variation in use of breast reconstruction in patients with breast cancer undergoing mastectomy in the United States. J Clin Oncol. 2014; 32(9):919-26.
41. Spear S L, Parikh P M, Reisin E, Menon N G. Acellular dermis-assisted breast reconstruction. Aesthetic Plast Surg. 2008; 32(3):418-25
42. JoAnna Nguyen T, Carey J N, Wong A K. Use of human acellular dermal matrix in implant-based breast reconstruction: evaluating the evidence. J Plast Reconstr Aesthet Surg. 2011; 64(12):1553-61.
43. Jones R E, Foster D S, Longaker M T. Management of Chronic Wounds-2018. JAMA. 2018; 320(14):1481-2.
44. Turner N J, Badylak S F. The Use of Biologic Scaffolds in the Treatment of Chronic Nonhealing Wounds. Adv Wound Care (New Rochelle). 2015; 4(8):490-500.
45. Chen S G, Tzeng Y S, Wang C H. Treatment of severe burn with DermACELL®, an acellular dermal matrix. Int J Burns Trauma. 2012; 2(2):105-9.
46. Wainwright D J. Use of an acellular allograft dermal matrix (AlloDerm) in the management of full thickness burns. Burns. 1995; 21(4):243-8.
47. Zelen C M, Orgill D P, Serena T, Galiano R, Carter M J, DiDomenico L A, Keller J, Kaufman J, Li W W. A prospective, randomised, controlled, multicentre clinical trial examining healing rates, safety and cost to closure of an acellular reticular allogenic human dermis versus standard of care in the treatment of chronic diabetic foot ulcers. Int Wound J. 2017; 14(2):307-15.
48. Cazzell S, Vayser D, Pham H, Walters J, Reyzelman A, Samsell B, Dorsch K, Moore M. A randomized clinical trial of a human acellular dermal matrix demonstrated superior healing rates for chronic diabetic foot ulcers over conventional care and an active acellular dermal matrix comparator. Wound Repair Regen. 2017; 25(3):483-97.
49. MacNeil S. Progress and opportunities for tissue-engineered skin. Nature. 2007; 445(7130):874-80.
50. Morris A H, Stamer D, Kyriakides T, editors. The host response to naturally-derived extracellular matrix biomaterials. Seminars in Immunology; 2017: Elsevier.
51. Shau D, Patton R, Patel S, Ward L, Guild G, 3rd. Synthetic mesh vs. allograft extensor mechanism reconstruction in total knee arthroplasty—A systematic review of the literature and meta-analysis. Knee. 2018; 25(1):2-7.
52. Purnell C A, Souza J M, Park E, Dumanian G A. Repair of recurrent hernia after biologic mesh failure in abdominal wall reconstruction. Am J Surg. 2014; 208(5):788-93.
53. Macadam S A, Lennox P A. Acellular dermal matrices: Use in reconstructive and aesthetic breast surgery. Can J Plast Surg. 2012; 20(2):75-89.
54. Fortier J L, Castiglione C L, Guo L. Skin Grafting. In: Orgill D P, editor. Interventional Treatment of Wounds: A Modern Approach for Better Outcomes. Cham: Springer International Publishing; 2018. p. 123-42.
55. Lee E I, Chike-Obi C J, Gonzalez P, Garza R, Leong M, Subramanian A, Bullocks J, Awad S S. Abdominal wall repair using human acellular dermal matrix: a follow-up study. Am J Surg. 2009; 198(5):650-7.
56. UniCare. Allogeneic, Xenographic, Synthetic and Composite Products for Wound Healing and Soft Tissue Grafting. Medical Policy. Medical Policy. 2018.
57. Foox M, Zilberman M. Drug delivery from gelatin-based systems. Expert Opin Drug Deliv. 2015; 12(9):1547-63.
58. Santoro M, Tatara A M, Mikos A G. Gelatin carriers for drug and cell delivery in tissue engineering. J Control Release. 2014; 190:210-8.
59. Yan L P, Oliveira J M, Oliveira A L, Reis R L. Core-shell silk hydrogels with spatially tuned conformations as drug-delivery system. J Tissue Eng Regen Med. 2017; 11(11):3168-77.
60. Oliveira I, Carvalho A L, Radhouani H, Goncalves C, Oliveira J M, Reis R L. Promising Biomolecules. Adv Exp Med Biol. 2018; 1059:189-205.
61. Yucel T, Lovett M L, Kaplan D L. Silk-based biomaterials for sustained drug delivery. J Control Release. 2014; 190:381-97.
62. Van Den Bulcke A I, Bogdanov B, De Rooze N, Schacht E H, Cornelissen M, Berghmans H. Structural and rheological properties of methacrylamide modified gelatin hydrogels. Biomacromolecules. 2000; 1(1):31-8.
63. Ovsianikov A, Deiwick A, Van Vlierberghe S, Dubruel P, Moller L, Drager G, Chichkov B. Laser fabrication of three-dimensional CAD scaffolds from photosensitive gelatin for applications in tissue engineering. Biomacromolecules. 2011; 12(4):851-8.
64. Liu X, Ma P X. Phase separation, pore structure, and properties of nanofibrous gelatin scaffolds. Biomaterials. 2009; 30(25):4094-103.
65. Rohanizadeh R, Swain M V, Mason R S. Gelatin sponges (Gelfoam) as a scaffold for osteoblasts. J Mater Sci Mater Med. 2008; 19(3):1173-82.
66. Jaipan P, Nguyen A, Narayan R J. Gelatin-based hydrogels for biomedical applications. MRS Communications. 2017; 7(3):416-26.
67. Horan R L, Toponarski I, Boepple H E, Weitzel P P, Richmond J C, Altman G H. Design and characterization of a scaffold for anterior cruciate ligament engineering. J Knee Surg. 2009; 22(1):82-92.
68. Wang Y, Rudym D D, Walsh A, Abrahamsen L, Kim H J, Kim H S, Kirker-Head C, Kaplan D L. In vivo degradation of three-dimensional silk fibroin scaffolds. Biomaterials. 2008; 29(24-25):3415-28.
69. von Hagens G, Tiedemann K, Kriz W. The current potential of plastination. Anat Embryol (Berl). 1987; 175(4):411-21.
70. Prasad G, Karkera B, Pandit S, Desai D, Tonse R G. Preservation of Tissue by Plastination: A Review. International Journal of Advanced Health Sciences. 2015; 1(11):27-31.
71. Riederer B M. Plastination and its importance in teaching anatomy. Critical points for long-term preservation of human tissue. J Anat. 2014; 224(3):309-15.
72. Latorre R M, Garcia-Sanz M P, Moreno M, Hernandez F, Gil F, Lopez O, Ayala M D, Ramirez G, Vazquez J M, Arencibia A, Henry R W. How useful is plastination in learning anatomy? J Vet Med Educ. 2007; 34(2):172-6.
73. Castano O, Perez-Amodio S, Navarro-Requena C, Mateos-Timoneda M A, Engel E. Instructive microenvironments in skin wound healing: Biomaterials as signal releasing platforms. Adv Drug Deliv Rev. 2018; 129:95-117.
74. Bonvillain R W, Scarritt M E, Pashos N C, Mayeux J P, Meshberger C L, Betancourt A M, Sullivan D E, Bunnell B A. Nonhuman primate lung decellularization and recellularization using a specialized large organ bioreactor. J Vis Exp. 2013(82):e50825.
75. Scarritt M E, Pashos N C, Motherwell J M, Eagle Z R, Burkett B J, Gregory A N, Mostany R, Weiss D J, Alvarez D F, Bunnell B A. Re-endothelialization of rat lung scaffolds through passive, gravity-driven seeding of segment-specific pulmonary endothelial cells. J Tissue Eng Regen Med. 2018; 12(2):e786-e806.
76. Pashos N C, Bunnell B A, inventors; The Administrators of the Tulane Educational Fund, assignee. Surgical Grafts for Replacing the Nipple and Areola or Damaged Epidermis. US patent US2018/0015204A1. Filed Oct. 31, 2015. Pub. Date Jan. 18, 2018.
77. Bonvillain R W, Danchuk S, Sullivan D E, Betancourt A M, Semon J A, Eagle M E, Mayeux J P, Gregory A N, Wang G, Townley I K, Borg Z D, Weiss D J, Bunnell B A. A nonhuman primate model of lung regeneration: detergent-mediated decellularization and initial in vitro recellularization with mesenchymal stem cells. Tissue Eng Part A. 2012; 18(23-24):2437-52.
78. Scarritt M E, Bonvillain R W, Burkett B J, Wang G, Glotser E Y, Zhang Q, Sammarco M C, Betancourt A M, Sullivan D E, Bunnell B A. Hypertensive rat lungs retain hallmarks of vascular disease upon decellularization but support the growth of mesenchymal stem cells. Tissue Eng Part A. 2014; 20(9-10):1426-43.
79. Hoganson D M, Owens G E, O'Doherty E M, Bowley C M, Goldman S M, Harilal D O, Neville C M, Kronengold R T, Vacanti J P. Preserved extracellular matrix components and retained biological activity in decellularized porcine mesothelium. Biomaterials. 2010; 31(27):6934-40.
80. Price A P, England K A, Matson A M, Blazar B R, Panoskaltsis-Mortari A. Development of a decellularized lung bioreactor system for bioengineering the lung: the matrix reloaded. Tissue Eng Part A. 2010; 16(8):2581-91.
81. Chisholm F, Varsou O. Resin-embedded anatomical cross-sections as a teaching adjunct for medical curricula: is this technique an alternative to potting and plastination? J Anat. 2018; 233(1):98-105.
82. Ottone N E, Baptista C A C, Latorre R, Bianchi H F, Del Sol M, Fuentes R. E12 sheet plastination: Techniques and applications. Clin Anat. 2018; 31(5):742-56.
83. Rabiei A A, Esfandiary E, Hajian M, Shamosi A, Mardani M, Rashidi B, Setayeshmehr M. Plastination of decalcified bone by a new resin technique. Adv Biomed Res. 2014; 3:18.
84. Soal S, Pollard M, Burland G, Lissaman R, Wafer M, Stringer M D. Rapid ultrathin slice plastination of embalmed specimens with minimal tissue loss. Clin Anat. 2010; 23(5):539-44.
85. Miklosova M, Miklos V. Plastination with silicone method S 10—monitoring and analysis causes of failure. Biomed Pap Med Fac Univ Palacky Olomouc Czech Repub. 2004; 148(2):237-8.
86. Liechty W B, Kryscio D R, Slaughter B V, Peppas N A. Polymers for drug delivery systems. Annu Rev Chem Biomol Eng. 2010; 1:149-73.
87. Shanbhag M S, Lathia J D, Mughal M R, Francis N L, Pashos N, Mattson M P, Wheatley M A. Neural progenitor cells grown on hydrogel surfaces respond to the product of the transgene of encapsulated genetically engineered fibroblasts. Biomacromolecules. 2010; 11(11):2936-43.
88. Sun J, Tan H. Alginate-Based Biomaterials for Regenerative Medicine Applications. Materials (Basel). 2013; 6(4):1285-309.
89. McCanless J D, Jennings L K, Bumgardner J D, Cole J A, Haggard W O. Hematoma-inspired alginate/platelet releasate/CaPO4 composite: initiation of the inflammatory-mediated response associated with fracture repair in vitro and ex vivo injection delivery. J Mater Sci Mater Med. 2012; 23(8):1971-81.
90. de Vos P, Spasojevic M, de Haan B J, Faas M M. The association between in vivo physicochemical changes and inflammatory responses against alginate based microcapsules. Biomaterials. 2012; 33(22):5552-9.
91. Vanacker J, Luyckx V, Dolmans M M, Des Rieux A, Jaeger J, Van Langendonckt A, Donnez J, Amorim C A. Transplantation of an alginate-matrigel matrix containing isolated ovarian cells: first step in developing a biodegradable scaffold to transplant isolated preantral follicles and ovarian cells. Biomaterials. 2012; 33(26):6079-85.
92. Silva N C, Silva S, Sarmento B, Pintado M. Chitosan nanoparticles for daptomycin delivery in ocular treatment of bacterial endophthalmitis. Drug Deliv. 2015; 22(7):885-93.
93. Tsai C H, Wang P Y, Lin I C, Huang H, Liu G S, Tseng C L. Ocular Drug Delivery: Role of Degradable Polymeric Nanocarriers for Ophthalmic Application. Int J Mol Sci. 2018; 19(9).
94. Zhang Y, Sun T, Jiang C. Biomacromolecules as carriers in drug delivery and tissue engineering. Acta Pharm Sin B. 2018; 8(1):34-50.
95. KnowledgeBase. Vancomycin (Vancocyn, Lyphocin) 2018. Available from: http://antibiotics.tokue.com/antimicrobial 1182 57.html.
96. Division of Product Quality Research, Office of Testing and Research, Center for Drug Evaluation and Research, Food and Drug Administration. Report to Office of Generic Drugs. Vancomycin Solubility Study. 2008.
97. American Society for Testing and Materials. ASTM D638-14, Standard Test Method for Tensile Properties of Plastics. West Conshohocken, Pa.: ASTM International; 2014.
98. Filipe E C, Santos M, Hung J, Lee B S L, Yang N, Chan A H P, Ng M K C, Rnjak-Kovacina J, Wise S G. Rapid Endothelialization of Off-the-Shelf Small Diameter Silk Vascular Grafts. JACC Basic Transl Sci. 2018; 3(1):38-53.
99. Pierce L M, Grunlan M A, Hou Y, Baumann S S, Kuehl T J, Muir T W. Biomechanical properties of synthetic and biologic graft materials following long-term implantation in the rabbit abdomen and vagina. Am J Obstet Gynecol. 2009; 200(5):549 el-8.
100. Musculoskeletal Transplant Foundation. DermaMatrix Acellular Dermis. Comparative testing. 2012.
101. Manschot J F, Brakkee A J. The measurement and modelling of the mechanical properties of human skin in vivo—I. The measurement. J Biomech. 1986; 19(7):511-5.
102. Ni Annaidh A, Bruyere K, Destrade M, Gilchrist M D, Ottenio M. Characterization of the anisotropic mechanical properties of excised human skin. J Mech Behav Biomed Mater. 2012; 5(1):139-48.
103. Hagihara M, Sutherland C, Nicolau D P. Development of HPLC methods for the determination of vancomycin in human plasma, mouse serum and bronchoalveolar lavage fluid. J Chromatogr Sci. 2013; 51(3):201-7.
104. Bartosova L, Bajgar J. Transdermal drug delivery in vitro using diffusion cells. Curr Med Chem. 2012; 19(27):4671-7.
105. Nowakowska J, Halkiewicz J, Lukasiak J W. TLC determination of selected macrocyclic antibiotics using normal and reversed phases. Chromatographia. 2002; 56(5-6):367-73.
106. British pharmacopoeia. London: British Pharmacopoeia Commission; 2002.
107. The United States Pharmacopeia. The National Formulary, 25th Ed. ed. Rockville: United States Pharmacopeial Convention; 2005.
108. Hudzicki J. Kirby-bauer disk diffusion susceptibility test protocol.: American Society for Microbiology; 2009. Available from: http://www.asmscience.org/content/education/protocol/protocol.3189.
109. Adams S B, Jr., Shamji M F, Nettles D L, Hwang P, Setton L A. Sustained release of antibiotics from injectable and thermally responsive polypeptide depots. J Biomed Mater Res B Appl Biomater. 2009; 90(1):67-74.
110. Stockert J C, Horobin R W, Colombo L L, Blazquez-Castro A. Tetrazolium salts and formazan products in Cell Biology: Viability assessment, fluorescence imaging, and labeling perspectives. Acta Histochem. 2018; 120(3):159-67.

### Example 6

This Example developing a polymer impregnation method, based upon tissue plastination, for generating a next generation acellular biologic graft. To date, plastination has not been used for the purpose of impregnating acellular biologic grafts with polymers for in vivo use in any therapeutic application.

To improve clinical outcomes for POP procedures that use either acellular biologic grafts or synthetic meshes, the inventors will impregnate an acellular biologic graft with biodegradable polymers that can enhance the mechanical properties of the graft and provide local, sustained drug delivery. This method will incorporate the polymer into the natural three-dimensional environment of the acellular tissue, which retains the extracellular matrix, including naturally aligned collagen fibers, shown to support recellularization and wound healing. Creation of a bioactive polymer-impregnated acellular biologic graft requires complete decellularization of donor tissue, physical encapsulation of antibiotic and anti-inflammatory agents in a biodegradable polymer, and impregnation of the decellularized skin with the drug-loaded polymer. Previously, the group has successfully decellularized a variety of tissues (lung, tumors, adipose, nipple-areolar complexes) including skin. In this Example, the inventors will develop a new polymer impregnation method and characterize the mechanical properties and cellular bioactivity of these grafts. Furthermore, the inventors will encapsulate compounds with these biodegradable polymers, prior to graft impregnation. This will allow the graft to release drug in a sustained manner at engraftment/surgical sites.

Broader Impacts:

POP is an area of significant unmet medical need, given the negative effects of untreated POP and the risks involved in POP surgical treatments. Worldwide, women have a 33-50% lifetime risk of developing POP. The product's end users are American women with symptomatic POP that seek a surgical solution, for example, roughly 300,000 such surgeries are performed each year in the US. Current surgical options for treating POP include synthetic mesh and acellular biologic graft implants. Synthetic meshes carry significant safety risks, and several countries have banned their use. Physicians have indicated that a biologic graft with greater safety and efficacy than current options would be completely transformative and gain widespread usage. It is estimated that the annual total addressable market for polymerimpregnated grafts for use in POP surgeries is $750 million.

Pelvic organ prolapse (POP) is a common condition in women caused by loss of pelvic floor tissue support, causing organs to herniate into the vaginal lumen or anus. POP symptoms, like incontinence, frequent urinary tract infections, difficult bowel movements, bleeding, pressure, and pain, can have significant negative impacts on women's health, daily living, and quality of life, especially impacting body image, family relationships, and sexual health1-3. POP affects 33% to 50% of all women4-6, potentially affecting over 1 billion women worldwide7. In the United States, over 300,000 POP surgeries are performed annually for symptomatic POP8. Surgical options for treating POP include synthetic mesh implants and acellular biologic grafts (ABGs). Synthetic meshes carry significant safety risks, including chronic infection, nerve and tissue damage, genital tearing, and others8-13. Current ABGs have fewer, usually less severe complications than synthetic meshes, but have higher recurrence rates and can have complications including infection, graft mechanical failure, and host-integration failure14-22. Despite the complications of current commercially available products for POP, surgeons still support their usage because synthetic meshes and ABGs have higher cure rates than traditional native tissue surgical methods, which fail within 2 years for 40% of women23-25. Given the negative effects of untreated POP and the risks involved in POP surgical treatment, POP represents an area of significant unmet medical need that is desperate for a safe and effective solution. Our target customers include urogynecologists and other decision-making physicians.

The inventors are developing a new type of ABG for POP surgeries. This graft will have enhanced mechanical properties and serve as a vehicle for delivery of drugs such as pain medication, antibiotics, and anti-infectives. This graft will address POP's unmet medical need, as it should be a safer, more effective product than synthetic meshes and ABGs currently on the market. A decellularization method 26 is being combined with a new bioactive polymer impregnation approach based on tissue plastination 27 to create a polymer-enhanced ABG. To date, plastination has not been used for the purpose of impregnating ABGs with polymers for in vivo use in any therapeutic application.

Tissue engineering approaches allow for the development of biologic substitutes that can replace or restore natural tissues. Specifically, we will use natural, biodegradable polymers engineered to release antibiotic, analgesic, or anti-inflammatory compounds, to prevent mechanical or immunological failure of an ABG. The polymers will enhance the mechanical properties of the ABG, namely elasticity and tensile strength, allowing it to be more durable than a non-impregnated ABG and, thus, prevent graft failure due to disruptive mechanical forces. These same polymers can encapsulate drugs for local release within the graft-surgical site. Release of antibiotic compounds can offset infection from opportunistic bacteria, improving healing and patient recovery time. Release of analgesic and anti-inflammatory compounds can decrease pain and local hyperinflammation, improving patient pain-management and increasing the likelihood of graft acceptance and host integration.

Here, the inventors discuss developing the plastination-inspired polymer impregnation method. We will then analyze the polymerimpregnated ABGs' physical, mechanical, and bioactive properties.

POP affects 33% to 50% of all women4-6, potentially affecting over 1 billion women worldwide7. Vaginal POP has a significant negative impact on women's health, daily living, and quality of life, especially impacting body image, family relationships, and sexual health1-3. Between 3% and 11% of all women have severe symptomatic POP4, 6, 28. This is approximately 3.5-12.8 million women in the US alone29. POP has non-surgical and surgical treatment options for pelvic floor repair. Non-surgical options (Kegel exercises, plastic pessaries, etc.) have limited efficacy for patients with severe POP. Surgical options include using native tissue, synthetic meshes, and ABGs. Over 40% of vaginal POP surgeries using native tissue will fail within two years24, 25. Synthetic meshes were created in response to high failure rates found with native tissue repairs13, 30. These are most commonly made of non-absorbable polypropylene8. ABGs were developed as an alternative to the polypropylene meshes30. ABGs are naturally derived from porcine or bovine dermal matrix that was made acellular and non-immunogenic through the process of decellularization. ABGs are ideal because they retain the native microenvironment of the extracellular matrix (ECM), which promotes host cell repopulation of the graft and, thus, complete integration of the graft with host tissue.

Currently available synthetic meshes and ABGs both have drawbacks. Synthetic mesh is the largest mass tort since asbestos, based on number of lawsuits (women have filed over 104,000 lawsuits in US federal court31). Several high-profile lawsuits have been filed over the use of synthetic meshes in POP surgeries, with women suffering serious side effects including death, chronic infection (e.g., sepsis), bleeding, nerve and tissue damage, perforated organs, vaginal scarring, genital tearing, pain during sex, chronic abdominal pain, and psychological distress that accompanies these physical side effects9, 13. An Australian Senate inquiry, in consultation with the Medical Board of Australia, determined that vaginal mesh usage for POP caused “historic agony and pain” that had a “devastating impact” on women's health, careers, and relationships9, 10, 32. In a high-profile US case in March 2018, Johnson & Johnson's Ethicon subsidiary lost a $35 million lawsuit for concealing failure, injury, and complication rates from patients. The suit claimed Ethicon's mesh product, Prolift, “caused severe and irreversible injuries, conditions, and damage to a significant number of women”33. Some companies have opted to settle out of court rather than go on public trial. Endo International, Inc., had a $830 million settlement in 2014 and an additional $400 million settlement later that same year34. The Food and Drug Administration (FDA) issued a Public Health Notification in 2008 about the risks of using synthetic vaginal mesh for POP12. By 2011, the FDA determined that serious adverse events associated with synthetic mesh in POP are not rare and issued an update on the mesh's safety and efficacy. The FDA ultimately decided that the safety of synthetic mesh for POP is not well established. In 2016, the FDA issued a final order reclassifying the meshes as Class III devices, requiring more extensive clinical trial data11. In 2017, the Australian Therapeutic Goods Administration cancelled the approval of several types of synthetic mesh in POP surgeries, effectively banning the products35. The National Institute for Health and Clinical Excellence in the United Kingdom issued a guidance statement that acts a de facto ban on synthetic vaginal mesh in POP surgeries36. Synthetic meshes are banned in all pelvic operations in New Zealand, not just vaginal POP36.

ABGs have fewer complications than synthetic meshes for POP14; however, they do have their own issues. Every ABG used for POP to date has shown higher recurrence rates than the synthetic meshes14. ABGs used in other indications have complication rates of up to 23%37-41. Complications are varied, and include infection, poor mechanical integrity, and failure to integrate15-22. Given the 33-50% lifetime risk of developing POP for women, the 12% lifetime incidence of POP surgery for women8, 28, 42-44, the negative effects of untreated POP, and the known risks involved in POP surgical treatment, POP represents an area of significant unmet medical need.

The inventors are developing a new type of ABG for use in POP surgeries for women with severe POP. Through tissue engineering practices, our polymer-impregnated ABG will have enhanced mechanical properties and will serve as vehicle for delivering drugs such as pain medication, antibiotics, and anti-infectives, to help decrease potential complications and improve the safety and efficacy of POP surgery.

The inventors discuss herein the development of a new polymer-impregnated ABG for use in severe POP surgeries, which will be significantly safer and more effective than existing treatment options:

Native Tissues:

Some transvaginal POP surgeries use the patient's native tissues. Native tissue repairs have discouraging rates of failure—40% of native tissue repairs fail within 2 years—and many surgeons prefer “reinforced or augmented repairs” using the two options below24, 25, 47-49.

Synthetic Mesh implants:

Synthetic meshes can be made of a variety of materials, most commonly non-absorbable polypropylene8. Serious concerns about the safety of synthetic vaginal meshes have been raised (see discussion herein).

ABGs:

ABGs are being explored as safer alternatives to mesh30. Clinicians are currently using Cook Medical's Surgisis® BioDesign™ anterior pelvic floor graft and Acell, Inc.'s MatriStem™ pelvic floor matrix14. ABGs not only repair POP, but also offer a scaffold for the regeneration of the patient's tissue. To date, every ABG has shown higher recurrence rates than the synthetic meshes, but with fewer complications14.

In POP, the vagina and supportive soft tissues are compromised both structurally and functionally, with these tissues showing disorganized and atrophied smooth muscle and altered collagen and elastin content30, 55-59. Physicians are turning to ABGs as safer options for POP repair over the use of synthetic meshes. ABGs are tissues, such as skin or tendon, that are recovered from a donor (human or animal) and processed to remove cells and immunogens. ECM proteins (e.g. collagen, elastin), which largely comprise the architecture of most tissues, are retained during decellularization. This process essentially generates a cell- and DNA-free three-dimensional graft that is amenable to host cell repopulation and remodeling during the course of healing.

The target non-autologous surgical material is one that (1) provides structural support, (2) fosters the body's natural wound-healing process, (3) allows for ingrowth, and (4) fully integrates with host tissue in time—all characteristics of ABGs. ABGs are superior to synthetic grafts in that they preserve, as oppose to recreate, the native matrix microenvironment, retaining the unique and complex matrix composition of the tissue20, 60. Preserving these complex properties promotes efficient repopulation and functional reestablishment of cells and blood supply within the ABGs, leading to their increased success over synthetic grafts. For over 30 years, ABGs have been used in surgeries for breast reconstruction, abdominal hernia repair, burn wound healing, urethra repair, tendon repair, and diabetic ulcer/chronic wound repair61, 62. Commercially available ABGs, like AlloDerm, DermACELL, and FlexHD, are considered medically necessary for breast reconstructions, abdominal surgical repairs, and burns, along with other indications.

Despite the advantages of ABGs, there is significant room for improvement in ABGs for all indications. Meta-analyses on large clinical studies for ABG use in breast reconstructions and hernia repairs show ABGs have relatively high complication rates, estimated at 10-23%37-41. Specific complications in patients with ABGs are most commonly attributed to infection (cellulitis), seroma formation, and necrosis15, 16. Closer analyses into ABG failures show reduced vascularization, poor mechanical integrity, failure to integrate (resulting in scarring), and immune rejection17-22.

To improve clinical outcomes for POP procedures and to develop new capabilities of ABGs for wider applications in regenerative medicine, the inventors will impregnate ABGs with biodegradable polymers that can provide enhanced mechanical properties and local, sustained release of drugs. Without being bound by theory, polymer impregnation of an ABG will itself enhance the mechanical properties of the graft, enabling it to be more durable than a non-impregnated graft and thus prevent graft failure due to mechanical forces. Infection, commonly a result of opportunistic bacteria, could be offset through sustained, local antibiotic release from the polymer, while the addition of anti-inflammatory and analgesic agents could reduce local inflammation and decrease pain at the surgical site. These additive properties will increase graft acceptance and host-integration, lessening the likelihood of complications and easing patient recovery.

The inventors describe here in this example the adaptation of tissue plastination techniques63-66 for strengthening ABGs and sustained, localized delivery of drugs from ABGs. Polymer embedded grafts have been described; however, these are usually a combination of synthetic or natural polymers and fabricated collagen matrices67; not naturally occurring ABGs. This method will force-impregnate polymers into the dense, native three-dimensional environment of an ABG, allowing for enhanced mechanical properties of the graft while retaining the morphological complexity of the endogenous ECM. Maintenance of the natural collagen fiber density and alignment within polymer-impregnated grafts will further provide support for robust recellularization and wound healing 67, 68.

There are three main aspects to the polymer impregnation of ABGs described herein: (1) decellularization of intact tissue, (2) plastination-based impregnation of natural polymers, and (3) drug release from polymers. Here, the inventors will integrate concepts (1) and (2). Subsequently, the inventors will then incorporate (3) drug release. Decellularization: The inventors have previously optimized decellularization techniques for whole organ regeneration of lung in rat and non-human primate (NHP) models69, 70, and have successfully employed these approaches on human skin. With modification of this decellularization process, the inventors have demonstrated feasibility of decellularizing dermal matrices71. We have shown that our decellularization process meets previously defined criteria for generating non-immunogenic acellular tissue post decellularization71, 72. For example, see US 2018/0015204 A1 (“Surgical Grafts for Replacing the Nipple and Areola or Damaged Epidermis”)26, which is incorporated by reference herein its entirety.

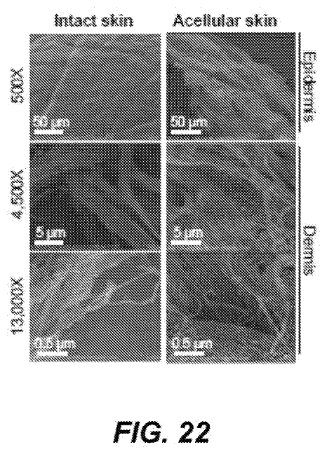

We have characterized our decellularization process on NHP dermis for the retention of ECM components collagen, glycosaminoglycans (GAGs), and elastin71. Briefly, collagens (types-I and -III) are major ECM components of skin, GAGs are hydrophilic polysaccharides that provide impact retention to the ECM, and elastin fibers are an essential component for skin elasticity. We confirmed preservation of collagen and GAG content to levels similar to intact dermis, and detected a decrease in elastin levels71. A decrease in elastin is highly common in decellularized tissue73, 74; however, the decreased elastin levels we measured are sufficient for the maintenance of native-like mechanical properties73, 74. The levels of retained ECM components from our decellularized NHP dermal matrix are similar to data from other epithelial tissue such as lung from rhesus macaque and rat, and skin from pig73-76. Furthermore, our decellularized dermis supported cell proliferation (>65%), cell migration, and did not significantly induce apoptosis (<1.5%)71. Additionally, we evaluated ECM fiber structure of our decellularized NHP dermis with scanning electron cryomicroscopy and found the integrity of collagen bundles and fibers were maintained in the dermal and epidermal layers, similar to intact dermis (FIG. 22). Our decellularized dermis is devoid of immunogenic levels of genomic material and retains its overall structure on the micro- and macroscale. These data demonstrate that our decellularization process is appropriately optimized for skin, creating an acellular matrix that retains structural elements.

Plastination:

Gunther von Hagens popularized plastination in the 1970s and the process has since been used to preserve surgical or autopsy tissue samples for teaching, histology, court evidence, and whole organism preservation64. The process is well-defined63-66 with the most well-known and well-characterized method of polymer impregnation for human tissue being the S10 Technique65, 66. In brief, this technique dehydrates fixed whole organs or organ slices through controlled incubations in organic solvents, including ethanol and acetone. Upon application of a vacuum, the acetone within a tissue undergoes a phase transition from liquid to gas, creating a negative pressure within the tissue that drives bath solutions, such as polymers, directly into the graft. This technique has been widely used with a range of polymers of various molecular sizes and charges, including silicones, polyesters, and resins77-81. To date, plastination has not been used for the purpose of impregnating ABGs with polymers for in vivo use in any therapeutic application.

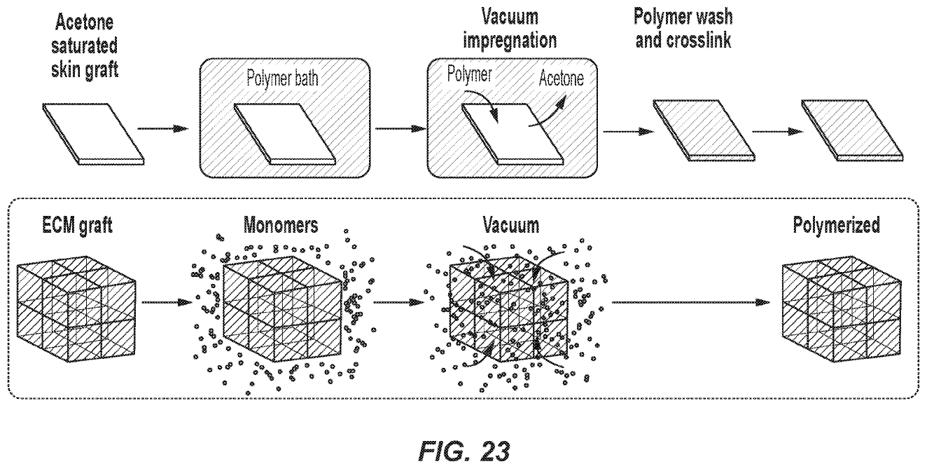

Improvements will be made to the standard S10 Plastination Technique. First, we will use our decellularization process described above to generate a native, non-crosslinked, ABG. After dehydration of the ABG through successive incubations in ethanol, the graft will be saturated with acetone. Similar to the original plastination process, we will use controlled vacuum driven force impregnation27 of the graft to replace the acetone in the graft with biomaterials and biocompatible biologically-derived polymers and proteins (FIG. 23).

Drug Release:

Polymers, including biodegradable polymers, have long been used in drug delivery systems as carriers82. We plan to utilize well established natural polymers that are amenable to drug encapsulation for localized drug delivery at sites of graft placement. We will leverage knowledge and methods of dermal delivery and sustained-release of drugs to develop a method to provide sustained drug release within regenerating tissue. This approach avoids systemic effects of current methods and provides a higher therapeutic dose at the relevant site of drug action. We have published on biodegradable polymers used to encapsulate and release growth factors in a controlled, sustained manner83, which is incorporated by reference in its entirety. This study used a an alginate construct, for example, as a multi-functional tissue scaffold for central nervous system repair that delivered a brain-derived neurotrophic factor (Neurotrophin-3)83. In our polymer-impregnated ABG program, we will use similar materials and methods to physically encapsulate antibiotics, analgesics, and/or anti-inflammatory agents in the polymers we select, to address issues with infection, pain, and host hyperinflammatory response.

Risks, along with some mitigation efforts, are summarized in the Table below.

| Risk | Description | Mitigation |
|---|---|---|
| Polymer- | Size of protein polymers | Use recombinant polymer forms that retain |
| impregnation | hinder penetration into tissue | polymerization to reduce monomer size |
| | Charge of polymers exclude | Solubilize polymers in different pH solutions or |
| | penetration into tissue | solvents to alter overall charge state of polymers |
| Mechanical | Non-desirable mechanical | Optimize polymer concentrations and include mild |
| properties | properties of tissue after | cross-linking reagents to covalently associate |
| | impregnation | polymers to matrix |
| Drug release | Temporally controlling release | Optimize polymer concentrations to allow for steady |
| (Phase II) | of drugs from polymeric | drug release properties |
| | encapsulation | |

Overview: POP is an area of high unmet medical need, given the 33-50% lifetime risk of developing POP for women and the 12% lifetime incidence of POP surgery for women8, 28, 42-44. None of the current surgical treatment options for severe POP are acceptable: native tissue replacements frequently fail within 2 years24, 25, synthetic meshes are unsafe11, 12, 35, 36, and current ABGs have high complication rates37-41. ABG product failures include poor mechanical integrity, immune rejection, and infection15-22. The inventors are developing a new type of ABG product that will address these problems, using our patented decellularization26 and plastination-based polymer-impregnation27 processes. To date, plastination has not been used for the purpose of impregnating ABGs with polymers for in vivo use in any therapeutic application.

Specifically, we are developing a method to impregnate an ABG with biodegradable polymers that have well-characterized, robust mechanical properties and are amenable to the encapsulation of drugs for local and controlled delivery of compounds at engraftment sites. A polymer-impregnated graft will have enhanced mechanical properties, enabling it to be more durable than a non-impregnated graft and will decrease the likelihood of complications associated with mechanical failure. Controlled antibiotic release from these polymers at engraftment sites can have the potential to stem complications with infection and release of anti-inflammatory agents can immediately quell inflammation, allowing for increased likelihood of graft acceptance/integration with the host. Creation of a bioactive polymer-impregnated ABG requires complete decellularization of biologically-derived intact tissue, physical encapsulation of antibiotic and anti-inflammatory agents in a biodegradable polymer, and impregnation of the decellularized skin with the drugloaded polymer.

(1) Determination of which parameters optimize polymer impregnation of ABGs.

We will develop methods for impregnating acellular skin grafts with three biodegradable polymers: alginate, elastin, and silk fibroin. We intend to test the three polymers at three concentrations each and will also assess three vacuum impregnation incubation times. For each combination of parameters, we will generate test grafts in triplicate. These grafts will be histologically analyzed for polymer penetration and distribution. Conditions will be determined that produce a polymer occupancy in the graft that is ≥5% of the graft area and have a polymer distribution such that normalized polymer occupancy is ±40% of the mean (for description, see Experimental Approach). Grafts that meet these success criteria will advance to Objectives 2 and 3 described below.

Rationale: Here, we will develop methods for impregnating acellular skin grafts with each of three biodegradable polymers: alginate, elastin, and silk fibroin. These well-characterized polymers were chosen for their biocompatibility, tunable mechanical properties, small and relatively simple monomeric form, ease of crosslinking to generate polymeric forms, biologically-derived nature, and proven use in biomedical applications. By impregnating ABGs with these polymers, we can manipulate and enhance the mechanical properties of ABGs. In principle, the impregnated polymer can occupy a percentage of the void/interstitial space within the ECM and can crosslink onto itself and/or the ECM. Because these polymers readily crosslink through chemical (pH, Ca2+) or physical (temperature, sonication) mechanisms, crosslinking can occur rapidly and efficiently without regard for graft size. Non-enzyme-based crosslinking circumvents limitations with enzyme diffusion and sustained activity within the density of the graft. Additional reasons for selecting these three polymers are as follows:

(a) Alginate:

Alginate (algin, alginic acid) is a natural polymer isolated from brown seaweed and is an FDA-approved polymer generally regarded as safe (GRAS)87. Alginate has been widely used in regenerative medicine and drug delivery, as it is histocompatible for human use and has minimal or negligible cytotoxicity88-91. As mentioned herein section, the team has previously used alginate as a tissue scaffold for drug release83. Alginate can be induced to form highly cross-linked hydrogels with multivalent cations (e.g. Ca2+)83.

(b) Elastin:

As discussed above in the Decellularization section, we have previously characterized a 69% reduction in elastin content in NHP acellular dermis from native dermis (n=3, p<0.01)71. These findings were similar to data from rhesus macaque lung, rat lung, and porcine dermis: elastin decrease during the decellularization process is a widely observed and characterized feature of detergent-based decellularization methods73-76. Recent studies looking at the addition of insoluble elastin in combination with collagen in a biomimetic cardiovascular tissue scaffold found that elastin markedly altered the mechanical and biological properties of the scaffold, improving durability without negatively affecting pore size or porosity92. Tropoelastin, the monomeric form of elastin, can be induced to crosslink with itself or collagen. The most commonly used elastin is α-elastin, which can be crosslinked by heat, repetitive sonication92, or pH change93.

(c) Silk Fibroin:

Silk is primarily composed of two proteins, fibroin and sericin. Sericin may trigger an immune response, but fibroin does not94. Silk has recently been explored as a drug delivery vehicle, drug stabilizer, and biological scaffold for tissue engineering94 and is an FDA approved biomaterial. Silk is of interest to biomedical engineers due to its favorable biocompatibility properties and unique mechanical attributes, including a well-defined nano- and micro-scale structure hierarchy, biocompatibility, noninflammatory by-products, and sterilization method compatibility94-96. The FDA has approved a variety of silk products for in vivo use, including sutures and scaffolds (e.g. Seri® Surgical Scaffold). Silk can be induced to crosslink with itself or collagen by heat or repetitive sonication94.

Experimental Approach:

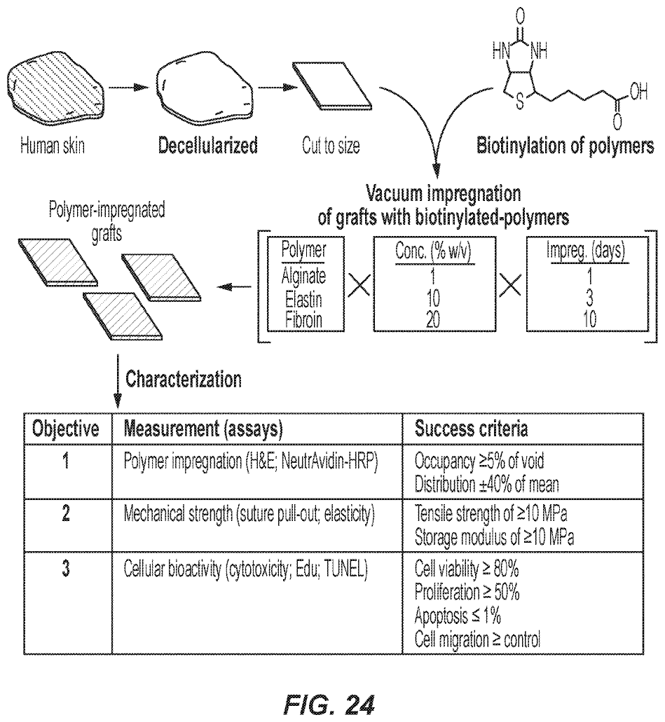

Polymer impregnation will be performed as described herein. FIG. 24 shows the work flow for this method. We will first decellularize human donor cadaver skin (2-3 mm thick) to serve as our model ABG. This study does not constitute human subjects research. All tissue is obtained through AATB-accredited tissue banks from deceased donors. Tissue banks are not associated with the inventors, the samples are not collected specifically for this study, and all samples are de-identified prior to transfer to the inventors.

Following decellularization and prior to polymer impregnation, ABGs will be cut into 1 cm×1 cm squares. In addition, all polymers will be biotinylated via standard carbodiimide-based crosslinking to allow polymer detection within ABGs. Biotinylation of the polymers is not expected to interfere with their ability to form polymers or have a significant effect on their physical properties. Biotin is a small 244 Da molecule that is widely used in conjugating proteins and has already been used with alginate97 and fibroin98 and shown not to alter polymer properties.

During impregnation, the ABG will be gradually dehydrated through stepwise incubations with ethanol before complete saturation with the organic solvent, acetone. The acetone-saturated ABG will be placed with a water solubilized polymer bath solution and subject to a low vacuum for different lengths of time at 4° C. Since polymerization increases under warmer temperatures, we will perform impregnations at 4° C. to ensure polymer entry into the ABG as a monomer before polymerization. For each polymer, we will test three concentrations: low (1% weight/volume), normal (10% weight/volume), and high (20% weight/volume). The amount of time needed for the vacuum-impregnation step can vary widely, depending on the thickness and complexity of the tissue being impregnated. Time frames longer than one day may be necessary at 4° C., thus we will be testing 1, 3, and 10-day incubation periods.

The goal of this step is to penetrate the polymer into and throughout the ABG. We will use a full factorial Design of Experiments approach to optimize polymer concentration, impregnation incubation time, and incubation temperature.

For each impregnation condition, we will generate impregnated grafts in triplicate for evaluation of impregnation efficacy. A total of 27 possible parameter combinations (three polymers; three concentrations; three times) will thus require generation of 81 grafts. We will submit samples for histological and immunohistochemical (IHC) analysis to measure polymer occupancy and distribution within the ABG. Polymer occupancy is defined as the polymer signal area (as measured by NeutrAvidin-HRP staining of biotinylated polymers) divided by the total ABG area. Because skin is stratified, the ECM density is not uniform across its thickness (epidermis to hypodermis). Thus, without being bound by theory, variability in polymer distribution across the thickness of the ABG may result. In order to measure polymer distribution, ABG area will be discretized into 500 μm×500 μm regions, normalized polymer occupancy will be calculated for each region, and then the distribution of normalized polymer occupancy throughout the graft analyzed for deviation from the mean. Normalized polymer occupancy is the polymer occupancy (i.e. % area polymer) divided by the interstitial space occupancy (i.e. % area void) prior to polymer impregnation. The interstitial space prior to impregnation will be measured directly from H&E stained sections (subtracting the ECM area from the total ABG area). We are normalizing polymer occupancy after impregnation to interstitial space occupancy prior to impregnation because it is our assumption that the amount of polymer that enters a region of the ABG is directly related to the amount of void space that existed prior to impregnation.

If the target polymer occupancy is not achieved, we will revisit the parameters and, using the data obtained from the initial set of experiments, analyze the impact and effect of each input factor as well as input factor interactions on polymer occupancy and distribution. We will continue to test our methodology via an iterative evolutionary operations (EVOL) experimental strategy for continuous improvement until we have identified effective impregnation conditions for each polymer.

If we have difficulties obtaining a sufficient quantity of donor skin from our tissue bank partners, for the purpose of determining the feasibility of creating a polymer-permeated ABG, we will utilize Bard's AlloMax™ collagen surgical graft, a widely-used commercially available ABG. We will make alternate arrangements and only use human skin decellularized as described herein.

Determining conditions that produce a polymer occupancy in the graft that is >5% of the graft area and have a polymer distribution such that normalized polymer occupancy is +40% of the mean. Without wishing to be bound by theory, a 5% increase of material within the graft will be sufficient to alter the mechanical properties. Normalized polymer occupancy should be within 40% of the mean based upon a Monte Carlo analysis for a discretized graft in which ECM occupancy and polymer occupancy for each region were randomly assigned within the ranges of 20-50% and 1-40%, respectively. Only grafts meeting these parameters will be used.

(2) Characterization of the Mechanical Properties of the Polymer-Impregnated ABGs.

We will test all polymer-impregnated acellular skin grafts described herein that meet the occupancy criteria described herein. We will correlate mechanical properties to impregnation method. Tests those established in the art which include, but are not limited to, tensile testing, ball-burst testing, and suture pull-out testing. Milestone: The polymer types(s) and conditions will be determined that are useful for optimize graft properties; for example, the target polymer-permeated graft has a Young's modulus and tensile strength≥10 MPa, which is greater than that of existing non-polymer-impregnated ABGs84-86.

This section addresses that a polymer impregnation of an ABG will enhance the mechanical properties of the graft, enabling it to be more durable than a non-impregnated graft. Without being bound by theory, an ideal polymer-impregnated ABG will have a Young's modulus and tensile strength of ≥10 MPa, based on literature values from excised and in vivo human skin as well as values for existing nonpolymer-impregnated ABGs84-86, 99, 100. We will examine those grafts that meet the polymer impregnation requirements from Section 1 (supra), and only those grafts with favorable characteristics will be used in the studies described below. If multiple time points for a given polymer/concentration pair advance from Section 1, only the test graft with the greatest polymer occupancy will advance. Therefore, a maximum of nine parameter combinations (in triplicate, for n=27 grafts) will advance into Objective 2 testing.

We will correlate mechanical properties with impregnation methodology. Data collected in this aim will also inform efforts to develop tunable, personalized grafts with different mechanical properties. Mechanical properties will be measured by uniaxial tensile, ball burst, and suture pull-out testing. Uniaxial tensile testing measures properties like Young's modulus (longitudinal stress:strain ratio to measure ability to withstand lengthwise tension or compression), ultimate tensile strength, yield strength, elongation, and Poisson's ratio. Ball burst testing is used to measure the resistance of a material to deformation and failure. Suture pull-out testing is commonly used to measure the resistance of a material, like surgical grafts and mesh, to failure at suture tie-in points.

Experimental Approach:

Uniaxial tensile, ball burst, and suture pull-out testing will be performed by Exponent, Inc. (Philadelphia, Pa.). For mechanical testing, polymer-impregnated grafts will be 6.4 cm×1 cm as needed for instrument fitting. Although larger than grafts used in Objective 1, since graft thickness will be consistent, we expect force-driven diffusion of polymer through the grafts to be consistent despite surface area differences. Commercially available ABGs will be included as controls for comparison.

(a) Unaxial Testing:

These tests will be performed according to ASTM D638101. Samples will be cut into a dog bone shape and the sample will be tested with a video extensometer to track strain. The elastic moduli, elongation at failure, and ultimate tensile strength will be determined.

(b) Ball Burst Testing:

These tests will be performed according to ASTM D6797-15102 and Freytes et al 103. Samples will be cut to 2×2 cm and ruptured in a probe ball burst pressure test with a 5.56 mm diameter probe and a 9.75 mm diameter clearance hole at a rate of 25.4 mm/min.

(c) Suture Pull-Out Testing:

Suture pull-out testing will be performed following ASTM F543104. A suture will be passed through the graft once, and the loose ends will be looped and tied around a suture pullout fixture. The skin graft will be stationary, gripped to the base of the load frame such that the direction of the suture's applied load will be parallel to the graft. A tensile load will be applied to the samples at 5 mm/min until failure occurs. The axial pullout strength of the test specimen will be determined.

If the above tests prove to be ineffective for determining the mechanical properties of the ABGs due to limited test conditions (i.e. temperature, pre-conditioning, strain rate) or suitability of tests for the material (i.e. elastic, viscoelastic, asymmetry), we will use dynamic mechanical analysis (DMA). DMA is used to measure polymer viscoelasticity by determining the complex modulus (stress:strain ratio under vibratory conditions), Young's modulus, and ultimate tensile strength. DMA is an alternative to uniaxial tensile and ball burst testing. If needed, a frequency sweep will be conducted on one specimen per sample at a fixed 20° C. temperature and varied frequencies between −0.1 Hz and 10 Hz, to determine the Young's, dynamic storage, and loss moduli, tensile strength, and tan(δ) as a function of frequency.

Determine which polymers and conditions optimize graft properties: the ideal polymer permeated graft has a Young's modulus and tensile strength of ≥10 MPa, which is greater than that of existing non-polymer-impregnated ABGs83-85.

(3) Characterize the In Vitro Bioactivity of the Polymer-Impregnated ABGs.

We will measure the bioactivity of the polymer-impregnated acellular skin grafts from Part 1 herein that met criteria by seeding grafts in vitro with human primary uterine fibroblasts and then determining percent cell viability, proliferation, and apoptosis. We will also assess the ability of cells to migrate into the graft.

Without wishing to be bound by theory, a polymer-impregnated ABG is bioactive and will support cell migration, which is essential for eventual clinical use. We will examine those grafts meeting the requirements from Section 1; only those grafts with favorable characteristics will be used in the studies described below. A maximum of nine parameter combinations (in triplicate, for n=27 grafts) will advance into Section 3 testing described herein. This is the only section discussing uses of human cells in combination with the graft.

We will measure the bioactivity of the polymer-impregnated grafts by seeding grafts in vitro with human primary uterine fibroblasts and then assessing cell proliferation, apoptosis, and migration. We previously performed similar assays after seeding NHP bone marrow-derived mesenchymal stem cells (BMSCs) onto decellularized NHP dermis71. These data showed less than 1% of cells being apoptotic and approximately 65% of the cell population remaining proliferative during the entirety of the study, with no observed changes between days71.

Cell viability staining will be performed. Microscopy work will be conducted by personnel at the Microscopy Services Laboratory, University of North Carolina—Chapel Hill. Ki-67, EdU, and TUNEL staining and image capture will be performed by HistoWiz (Brooklyn, N.Y.). The inventors will perform all analyses. For all of the bioactivity assays, statistical significance will be determined with two tailed ANOVA with α=0.05. Numerical results will be given as average±standard error of the mean.

Experimental Approach:

Acellular grafts will be pre-conditioned with cell growth media in a cell culture incubator under standard mammalian cell culture conditions (37° C./5% carbon dioxide) for 30 minutes prior to seeding. Each graft will be 1 cm×1 cm and seeded with 1×106 primary uterine fibroblasts; each graft will be run in triplicate. This density should provide sufficient infiltration of the graft so as to permit sufficient cell presence within the graft as well as good single cell resolution without a high background signal due to too many cells. The seeded grafts will be cultured for one and two weeks prior to analysis. For all stains, each seeded graft will be cut to 5 mm×5 mm pieces to avoid insufficient antibody and dye penetration into the tissue. Cell viability, proliferation, and apoptosis will be measured via fluorescence microscopy and quantified using ImageJ software. Measurements will be taken from 5 random fields of view per sample for each bioactivity assay and background subtracted. Out-of-focused signals of cells not in the focal plane will be excluded. Seeded grafts will be assessed as follows:

(a) Cell Viability:

Viability will be measured within the graft by calcein-AM and ethidium homodimer-1 staining (Thermo; LIVE/DEAD assay). Calcein-AM stains green living cells with active esterase activity, while ethidium homodimer-1 stains red the nuclei of cells with membrane integrity loss.

(b) Proliferation:

Cell proliferation will be measured through Ki-67 staining and EdU (5-ethynyl-2′-deoxyuridine) incorporation. IHC will be performed on sections of seeded grafts with anti-Ki67 (Cell Signaling, Mouse) and Alexa Fluor 488 conjugated secondary staining. The Click-iT EdU assay (Invitrogen) will label mitotically active cells through incorporation of fluorescently label EdU.

(c) Apoptosis:

Apoptosis will be assessed by the TdT-mediated dUTP Nick-end Labeling (TUNEL) assay (In Situ Cell Death Detection Kit—Fluorescein, Roche) as previously described71, 73. Samples treated with labeled solution without enzyme will be included as negative controls while samples treated with DNase I prior to TUNEL staining will serve as positive controls. All samples will be stained with DAPI after TUNEL staining.

(d) Cell Migration:

Using the slide images captured in the histology work described above, we will assess the ability of cells to migrate into the polymer-impregnated ABGs as compared to non-polymer treated ABGs. We will count relative number of cells, as indicated by stained nuclei, within each graft.

The cell migration study will quantify the number of cells per field as a function of distance from the graft edge in order to generate a “migration” value. However, fibroblasts seeded on a single side of the graft will presumably migrate into the graft on that single side. The majority of the migrating cells will be from the area where the graft makes direct contact with the cell culture plate, and distance migrated will be skewed depending on the original position of the migrating cells. For this reason, simply the number of cells migrated will be counted. We will be able to make more quantitative assessments of migration distance when the graft is implanted in an in vivo model.

We are developing polymer-impregnated ABGs to improve mechanical function of ABGs and deliver drugs locally in a controlled manner to enhance healing and patient outcomes. The Example herein describes efforts to produce a polymer-impregnated acellular skin graft. The inventors will mix selected drugs with the biodegradable polymer prior to graft impregnation. By choosing tunable biodegradable materials that are meant to break down within the body over time, we will allow for sustained drug delivery to the area surrounding the graft. We will characterize encapsulation efficacy and drug-release kinetics as well as graft genotoxicity.

Determine which polymers and conditions optimize graft properties: the ideal polymer permeated graft supports≥80% viable cells and ≥50% proliferative cells.

The Example describes herein critical path activities that will be developed for methods for impregnating ABGs with biodegradable polymers to improve the mechanical strength of ABGs and to allow for sustained, localized delivery of drugs. These activities will advance the proposed polymer-impregnated ABG program from concept stage to prototype stage.

### REFERENCES

1. Lowenstein L, Gamble T, Sanses T V, van Raalte H, Carberry C, Jakus S, Kambiss S, McAchran S, Pham T, Aschkenazi S, Hoskey K, Kenton K, Fellow's Pelvic Research N. Sexual function is related to body image perception in women with pelvic organ prolapse. J Sex Med. 2009; 6(8):2286-2291.
2. Fritel X, Varnoux N, Zins M, Breart G, Ringa V. Symptomatic pelvic organ prolapse at midlife, quality of life, and risk factors. Obstet Gynecol. 2009; 113(3):609-616.
3. Chan S S, Cheung R Y, Yiu K W, Lee L L, Pang A W, Chung T K. Symptoms, quality of life, and factors affecting women's treatment decisions regarding pelvic organ prolapse. Int Urogynecol J.2012; 23(8):1027-1033.
4. Barber M D, Maher C. Epidemiology and outcome assessment of pelvic organ prolapse. Int Urogynecol J. 2013; 24(11):1783-1790.
5. Aytan H, Ertunc D, Tok E C, Yasa O, Nazik H. Prevalence of pelvic organ prolapse and relatedfactors in a general female population. Turk J Obstet Gynecol. 2014; 11(3):176-180.
6. Barber M D. Pelvic organ prolapse. BMJ. 2016; 354. doi: 10.1136/bmj.i3853.
7. “World Population 2018.” from https://countrymeters.info/en/World.
8. US Food and Drug Administration. Urogynecologic Surgical Mesh: Update on the Safety and Effectiveness of Transvaginal Placement for Pelvic Organ Prolapse. 2011.
9. BBC News (2018). “Vaginal mesh implants: Australia apologises for ‘decades of pain’.” from https://www.bbc.com/news/world-australia-45806324.
10. Scott S, Branley A (2018). “Mesh implants: Government issues national apology over ‘agony and pain’ caused by device.” from https://www.abc.net.au/news/2018-10-10/mesh-implantsgovernment-issues-apology-to-women/10355546?section=health.
11. Kux L. Effective Date of Requirement for Premarket Approval for Surgical Mesh for Transvaginal Pelvic Organ Prolapse Repair. In: Food and Drug Administration, editor. Federal Register 2016. p. 364-370.
12. Schultz D G. FDA Public Health Notification: Serious Complications Associated with Transvaginal Placement of Surgical Mesh in Repair of Pelvic Organ Prolapse and Stress Urinary Incontinence. In: Center for Devices and Radiological Health, Food and Drug Administration, editors. 2008.
13. Campbell P, Jha S, Cutner A. Vaginal mesh in prolapse surgery. The Obstetrician & Gynaecologist. 2018; 20:49-56.
14. Margie Kahn. Personal Communication. 2018.
15. Weichman K E, Wilson S C, Weinstein A L, Hazen A, Levine J P, Choi M, Karp N S. The use of a cellular dermal matrix in immediate two-stage tissue expander breast reconstruction. Plast Reconstr Surg. 2012; 129(5): 1049-1058.
16. Chun Y S, Verma K, Rosen H, Lipsitz S, Morris D, Kenney P, Eriksson E. Implant-based breast reconstruction using acellular dermal matrix and the risk of postoperative complications. Plast Reconstr Surg. 2010; 125(2):429-436.
17. Vig K, Chaudhari A, Tripathi S, Dixit S, Sahu R, Pillai S, Dennis V A, Singh S R. Advances in SkinRegeneration Using Tissue Engineering. Int J Mol Sci. 2017; 18(4).
18. MacNeil S. Progress and opportunities for tissue-engineered skin. Nature. 2007; 445(7130):874-880.
19. Morris A H, Stamer D, Kyriakides T, editors. The host response to naturally-derived extracellular matrix biomaterials. Seminars in Immunology; 2017: Elsevier.
20. Shau D, Patton R, Patel S, Ward L, Guild G, 3rd. Synthetic mesh vs. allograft extensor mechanism reconstruction in total knee arthroplasty—A systematic review of the literature and meta-analysis. Knee. 2018; 25(1):2-7.
21. Purnell C A, Souza J M, Park E, Dumanian G A. Repair of recurrent hernia after biologic mesh failure in abdominal wall reconstruction. Am J Surg. 2014; 208(5):788-793.
22. Macadam S A, Lennox P A. Acellular dermal matrices: Use in reconstructive and aesthetic breast surgery. Can J Plast Surg. 2012; 20(2):75-89.
23. Future Market Insights. “Female Pelvic Implants Market: Companies to Focus on Reducing Cost of Implants to Gain Maximum Market Share: Global Industry Analysis 2012-2016 and Opportunity Assessment 2017-2027.” Market Report. 2018
24. Barber M D, Brubaker L, Burgio K L, Richter H E, Nygaard I, Weidner A C, Menefee S A, Lukacz E S, Norton P, Schaffer J, Nguyen J N, Borello-France D, Goode P S, Jakus-Waldman S, Spino C, Warren L K, Gantz M G, Meikle S F, Eunice Kennedy Shriver National Institute of Child Health, Human Development Pelvic Floor Disorders Network. of 2 transvaginal surgical approaches and perioperative behavioral therapy for apical vaginal prolapse: the OPTIMAL randomized trial. JAMA. 2014; 311(10):1023-1034
25. Siff L N, Barber M D. Native Tissue Prolapse Repairs: Comparative Effectiveness Trials. Obstet Gynecol Clin North Am. 2016; 43(1):69-81.
26. Pashos N C, Bunnell B A, inventors; The Administrators of the Tulane Educational Fund, assignee. Surgical Grafts for Replacing the Nipple and Areola or Damaged Epidermis. US patent US2018/0015204A1. Filed Oct. 31, 2015. Pub. Date Jan. 18, 2018.
27. Pashos N C, Heim W M, inventors; BioAesthetics Corporation, assignee. Polymer-PermeatedG rafts and Methods of Making and Using the Same. patent U.S. Provisional Patent Applicaton 62/651,485. Filed Apr. 2, 2018.
28. Transparency Market Research. “Pelvic Organ Prolapse Devices Market—Global Industry Analysis, Size, Share, Growth, Trends and Forecast 2018-2026.” Market Report. 201829.Economics and Statistics Administration, US Census Bureau, US Department of Commerce. Age and Sex Composition: 2010. 2011.
30. Liang R, Knight K, Easley D, Palcsey S, Abramowitch S, Moalli P A. Towards rebuilding vaginal support utilizing an extracellular matrix bioscaffold. Acta Biomater. 2017; 57:324-333.
31. Pelley S (2018). “Gynecological Mesh: The Medical Device that has 100,000 Women Suing.” CBS 60 Minutes. from https://www.cbsnews.com/news/boston-scientific-gynecological-mesh-themedical-device-that-has-100000-women-suing/.
32. Australia Co. Number of women in Australia who have had transvaginal mesh implants and related matters. 2018.
33. Perriello B (2018). “Johnson & Johnson's Ethicon loses $35 m pelvic mesh lawsuit.” from https://www.massdevice.com/johnson-johnsons-ethicon-loses-35m-pelvic-mesh-lawsuit/.
34. York M A (2018). “Ethicon, Inc. and J&J facing thousands of TVM and hernia mesh lawsuits: will they settle sooner or later?”. from https://www.masstortnexus.com/mass-torts-news/ethicon-incand-jj-facing-thousands-of-tvm-and-hernia-mesh-lawsuits-will-they-settle-sooner-or-later/.
35. Therapeutic Goods Administration (2017). “TGA undertakes regulatory actions after review intourogynaecological surgical mesh implants.” from https://www.tga.gov.au/alert/tga-actions-afterreview-urogynaecological-surgical-mesh-implants.
36. Ismail S. Three countries ban the use of vaginal mesh in prolapse surgery. International Urogynecological Association 2018; 12(4).
37. Kim J Y, Davila A A, Persing S, Connor C M, Jovanovic B, Khan S A, Fine N, Rawlani V. A meta analysis of human acellular dermis and submuscular tissue expander breast reconstruction. Plast Reconstr Surg. 2012; 129(1):28-41.
38. Ho G, Nguyen T J, Shahabi A, Hwang B H, Chan L S, Wong A K. A systematic review and meta analysis of complications associated with acellular dermal matrix-assisted breast reconstruction. Ann Plast Surg. 2012; 68(4):346-356.
39. Loo Y L, Kamalathevan P, Ooi P S, Mosahebi A. Comparing the Outcome of Different Biologically Derived Acellular Dermal Matrices in Implant-based Immediate Breast Reconstruction: A Meta analysis of the Literatures. Plast Reconstr Surg Glob Open. 2018; 6(3):e1701.
40. Trippoli S, Caccese E, Tulli G, Ipponi P, Marinai C, Messori A. Biological meshes for abdominal hernia: Lack of evidence-based recommendations for clinical use. Int J Surg. 2018; 52:278-284.
41. Slater N J, van der Kolk M, Hendriks T, van Goor H, Bleichrodt R P. Biologic grafts for ventral hernia repair: a systematic review. Am J Surg. 2013; 205(2):220-230.
42. Lawrence J M, Lukacz E S, Nager C W, Hsu J W, Luber K M. Prevalence and co-occurrence of pelvic floor disorders in community-dwelling women. Obstet Gynecol. 2008; 111(3):678-685.
43. Samuelsson E C, Victor F T, Tibblin G, Svardsudd K F. Signs of genital prolapse in a Swedish population of women 20 to 59 years of age and possible related factors. Am J Obstet Gynecol. 1999; 180(2 Pt 1):299-305.
44. Wu J M, Matthews C A, Conover M M, Pate V, Jonsson Funk M. Lifetime risk of stress urinary incontinence or pelvic organ prolapse surgery. Obstet Gynecol. 2014; 123(6):1201-1206.
45. Market Reports World. “Global Pelvic Organ Prolapse Repair Device Market Report 2017.” Market Report. 2018
46. Brown L K, Fenner D E, Berger M B, Delancey J O, Morgan D M, Patel D A, Schimpf M O. Defining patients' knowledge and perceptions of vaginal mesh surgery. Female Pelvic Med Reconstr Surg.2013; 19(5):282-287.
47. Weber A M, Walters M D, Piedmonte M R, Ballard L A. Anterior colporrhaphy: a randomized trial of three surgical techniques. Am J Obstet Gynecol. 2001; 185(6):1299-1304; discussion 1304-1296.
48. Olsen A L, Smith V J, Bergstrom J O, Colling J C, Clark A L. Epidemiology of surgically managed pelvic organ prolapse and urinary incontinence. Obstet Gynecol. 1997; 89(4):501-506.
49. Kontogiannis S, Goulimi E, Giannitsas K. Reasons for and Against Use of Non-absorbable, Synthetic Mesh During Pelvic Organ Prolapse Repair, According to the Prolapsed Compartment. Adv Ther. 2017; 33(12):2139-2149.
50. “Pelvic Organ Prolapse Repair Device Market Analysis, Market Size, Application Analysis, Regional Outlook, Competitive Strategies, and Forecasts, 2018-2025” Market Report. Market Expertz, 2018
51. Patrick J. Culligan C S, Christa Lewis, Troy D. Abell Cost-effectiveness analysis comparing robotic sacrocolpopexy to a vaginal mesh hysteropexy for treatment of uterovaginal prolapse. Open Journal of Obstetrics and Gynecology. 2013; 3:613-620.
52. “Wound Care Market and Market Share Analysis.” Market Report. Kalorama, 2017
53. “The Worldwide Market for Advanced Wound Care Products.” Market Report. Kalorama, 2018
54. Donate Life America (2018). “Tissue Donation: Everything You Need to Know.” fromhttps://www.donatelife.net/types-of-donation/tissue-donation/.
55. Bildircin D, Kokcu A, Celik H, Sagir D, Kefeli M. Comparison of connective tissue components in the uterine ligaments between women with and without pelvic organ prolapse. Minerva Ginecol.2014; 66(2):201-208.
56. Chen B, Yeh J. Alterations in connective tissue metabolism in stress incontinence and prolapse. J Urol. 2011; 186(5):1768-1772.
57. Meijerink A M, van Rijssel R H, van der Linden P J. Tissue composition of the vaginal wall in women with pelvic organ prolapse. Gynecol Obstet Invest. 2013; 75(1):21-27.
58. Boreham M K, Miller R T, Schaffer J I, Word R A. Smooth muscle myosin heavy chain andcaldesmon expression in the anterior vaginal wall of women with and without pelvic organprolapse. Am J Obstet Gynecol. 2001; 185(4):944-952.
59. Boreham M K, Wai C Y, Miller R T, Schaffer J I, Word R A. Morphometric analysis of smooth muscle in the anterior vaginal wall of women with pelvic organ prolapse. Am J Obstet Gynecol.2002; 187(1):56-63.
60. Lintin L A, Kingsnorth A N. Mechanical failure of a lightweight polypropylene mesh. Hernia.2014; 18(1):131-133.
61. Jeffcoate W J, Vileikyte L, Boyko E J, Armstrong D G, Boulton A J M. Current Challenges andOpportunities in the Prevention and Management of Diabetic Foot Ulcers. Diabetes Care.2018; 41(4):645-652.
62. Scarritt M E, Pashos N C, Bunnell B A. A review of cellularization strategies for tissue engineering of whole organs. Front Bioeng Biotechnol. 2015; 3:43.
63. von Hagens G, Tiedemann K, Kriz W. The current potential of plastination. Anat Embryol (Berl).1987; 175(4):411-421.
64. Prasad G, Karkera B, Pandit S, Desai D, Tonse R G. Preservation of Tissue by Plastination: A Review. International Journal of Advanced Health Sciences. 2015; 1(11):27-31.
65. Riederer B M. Plastination and its importance in teaching anatomy. Critical points for long-term preservation of human tissue. J Anat. 2014; 224(3):309-315.
66. Latorre R M, Garcia-Sanz M P, Moreno M, Hernandez F, Gil F, Lopez O, Ayala M D, Ramirez G, Vazquez J M, Arencibia A, Henry R W. How useful is plastination in learning anatomy? J Vet MedEduc. 2007; 34(2):172-176.
67. Castano O, Perez-Amodio S, Navarro-Requena C, Mateos-Timoneda M A, Engel E. Instructive microenvironments in skin wound healing: Biomaterials as signal releasing platforms. Advanced drug delivery reviews. 2018; 129:95-117. Epub 2018/04/09.
68. He C, Yang Z, Jin Y, Qi X, Chu J, Deng X. ADM Scaffolds Generate a Pro-regenerative Microenvironment During Full-Thickness Cutaneous Wound Healing Through M2 Macrophage Polarization via Lamtor1. Frontiers in physiology. 2018; 9:657. Epub 2018/06/20.
69. Bonvillain R W, Scarritt M E, Pashos N C, Mayeux J P, Meshberger C L, Betancourt A M, Sullivan D E, Bunnell B A. Nonhuman primate lung decellularization and recellularization using a specialized large-organ bioreactor. J Vis Exp. 2013(82):e50825.
70. Scarritt M E, Pashos N C, Motherwell J M, Eagle Z R, Burkett B J, Gregory A N, Mostany R, Weiss D J, Alvarez D F, Bunnell B A. Re-endothelialization of rat lung scaffolds through passive, gravity driven seeding of segment-specific pulmonary endothelial cells. J Tissue Eng Regen Med.2018; 12(2):e786-e806.
71. Pashos N C, Scarritt M E, Eagle Z R, Gimble J M, Chaffin A E, Bunnell B A. Characterization of an Acellular Scaffold for a Tissue Engineering Approach to the Nipple-Areolar Complex Reconstruction. Cells Tissues Organs. 2017; 203(3):183-193.
72. Crapo P M, Gilbert T W, Badylak S F. An overview of tissue and whole organ decellularization processes. Biomaterials. 2011; 32(12):3233-3243.
73. Bonvillain R W, Danchuk S, Sullivan D E, Betancourt A M, Semon J A, Eagle M E, Mayeux J P, Gregory A N, Wang G, Townley I K, Borg Z D, Weiss D J, Bunnell B A. A nonhuman primate model of lung regeneration: detergent-mediated decellularization and initial in vitro recellularization with mesenchymal stem cells. Tissue Eng Part A. 2012; 18(23-24):2437-2452.
74. Scarritt M E, Bonvillain R W, Burkett B J, Wang G, Glotser E Y, Zhang Q, Sammarco M C, Betancourt A M, Sullivan D E, Bunnell B A. Hypertensive rat lungs retain hallmarks of vascular disease upon decellularization but support the growth of mesenchymal stem cells. Tissue Eng Part A. 2014; 20(9-10):1426-1443.
75. Hoganson D M, Owens G E, O'Doherty E M, Bowley C M, Goldman S M, Harilal D O, Neville C M, Kronengold R T, Vacanti J P. Preserved extracellular matrix components and retained biological activity in decellularized porcine mesothelium. Biomaterials. 2010; 31(27):6934-6940.
76. Price A P, England K A, Matson A M, Blazar B R, Panoskaltsis-Mortari A. Development of a decellularized lung bioreactor system for bioengineering the lung: the matrix reloaded. Tissue Eng Part A. 2010; 16(8):2581-2591. doi: 10.1089/ten.TEA.2009.0659.
77. Chisholm F, Varsou O. Resin-embedded anatomical cross-sections as a teaching adjunct for medical curricula: is this technique an alternative to potting and plastination? J Anat.2018; 233(1):98-105.
78. Ottone N E, Baptista C A C, Latorre R, Bianchi H F, Del Sol M, Fuentes R. E12 sheet plastination: Techniques and applications. Clin Anat. 2018; 31(5):742-756.
79. Rabiei A A, Esfandiary E, Hajian M, Shamosi A, Mardani M, Rashidi B, Setayeshmehr M. Plastination of decalcified bone by a new resin technique. Adv Biomed Res. 2014; 3:18.
80. Soal S, Pollard M, Burland G, Lissaman R, Wafer M, Stringer M D. Rapid ultrathin slice plastination of embalmed specimens with minimal tissue loss. Clin Anat. 2010; 23(5):539-544.
81. Miklosova M, Miklos V. Plastination with silicone method S 10—monitoring and analysis causes of failure. Biomed Pap Med Fac Univ Palacky Olomouc Czech Repub. 2004; 148(2):237-238.
82. Liechty W B, Kryscio D R, Slaughter B V, Peppas N A. Polymers for drug delivery systems. AnnuRev Chem Biomol Eng. 2010; 1:149-173.
83. Shanbhag M S, Lathia J D, Mughal M R, Francis N L, Pashos N, Mattson M P, Wheatley M A. Neural progenitor cells grown on hydrogel surfaces respond to the product of the transgene of encapsulated genetically engineered fibroblasts. Biomacromolecules. 2010; 11(11):2936-2943.
84. Filipe E C, Santos M, Hung J, Lee B S L, Yang N, Chan A H P, Ng M K C, Rnjak-Kovacina J, Wise S G. Rapid Endothelialization of Off-the-Shelf Small Diameter Silk Vascular Grafts. JACC Basic Transl Sci. 2018; 3(1):38-53.
85. Pierce L M, Grunlan M A, Hou Y, Baumann S S, Kuehl T J, Muir T W. Biomechanical properties of synthetic and biologic graft materials following long-term implantation in the rabbit abdomen and vagina. Am J Obstet Gynecol. 2009; 200(5):549 e541-548.
86. Musculoskeletal Transplant Foundation. DermaMatrix Acellular Dermis. Comparative testing. 2012.
87. US Food and Drug Administration. CFR—Code of Federal Regulations Title 21. 2018.
88. Sun J, Tan H. Alginate-Based Biomaterials for Regenerative Medicine Applications. Materials(Basel). 2013; 6(4):1285-1309.
89. McCanless J D, Jennings L K, Bumgardner J D, Cole J A, Haggard W O. Hematoma-inspired alginate/platelet releasate/CaPO4 composite: initiation of the inflammatory-mediated response associated with fracture repair in vitro and ex vivo injection delivery. J Mater Sci Mater Med. 2012; 23(8):1971-1981.
90. de Vos P, Spasojevic M, de Haan B J, Faas M M. The association between in vivo physicochemical changes and inflammatory responses against alginate based microcapsules. Biomaterials. 2012; 33(22):5552-5559.
91. Vanacker J, Luyckx V, Dolmans M M, Des Rieux A, Jaeger J, Van Langendonckt A, Donnez J, Amorim C A. Transplantation of an alginate-matrigel matrix containing isolated ovarian cells: first step in developing a biodegradable scaffold to transplant isolated preantral follicles and ovarian cells. Biomaterials. 2012; 33(26):6079-6085.
92. Ryan A J, O'Brien F J. Insoluble elastin reduces collagen scaffold stiffness, improves viscoelastic properties, and induces a contractile phenotype in smooth muscle cells. Biomaterials. 2015; 73:296-307.
93. Mithieux S M, Tu Y, Korkmaz E, Braet F, Weiss A S. In situ polymerization of tropoelastin in the absence of chemical cross-linking. Biomaterials. 2009; 30(4):431-435.
94. Yucel T, Lovett M L, Kaplan D L. Silk-based biomaterials for sustained drug delivery. J Control Release. 2014; 190:381-397.
95. Horan R L, Toponarski I, Boepple H E, Weitzel P P, Richmond J C, Altman G H. Design and characterization of a scaffold for anterior cruciate ligament engineering. J Knee Surg.2009; 22(1):82-92.
96. Wang Y, Rudym D D, Walsh A, Abrahamsen L, Kim H J, Kim H S, Kirker-Head C, Kaplan D L. In vivo degradation of three-dimensional silk fibroin scaffolds. Biomaterials. 2008; 29(24-25):3415-3428.
97. Polyak B, Geresh S, Marks R S. Synthesis and characterization of a biotin-alginate conjugate and its application in a biosensor construction. Biomacromolecules. 2004; 5(2):389-396.
98. Wang X, Kaplan D L. Functionalization of silk fibroin with NeutrAvidin and biotin. Macromol Biosci.2011; 11(1):100-110.
99. Manschot J F, Brakkee A J. The measurement and modelling of the mechanical properties of human skin in vivo—I. The measurement. J Biomech. 1986; 19(7):511-515.
100. Ni Annaidh A, Bruyere K, Destrade M, Gilchrist M D, Ottenio M. Characterization of the anisotropic mechanical properties of excised human skin. J Mech Behav Biomed Mater. 2012; 5(1):139-148.
101. American Society for Testing and Materials. ASTM D638-14, Standard Test Method for Tensile Properties of Plastics. West Conshohocken, Pa.: ASTM International; 2014.
102. American Society for Testing and Materials. ASTM D6797-15, Standard Test Method for Bursting Strength of Fabrics Constant-Rate-of-Extension (CRE) Ball Burst Test. West Conshohocken, Pa.: ASTM International; 2015.
103. Freytes D O, Rundell A E, Vande Geest J, Vorp D A, Webster T J, Badylak S F. Analytically derived material properties of multilaminated extracellular matrix devices using the ball-burst test. Biomaterials. 2005; 26(27):5518-5531.
104. American Society for Testing and Materials. ASTM F543-17, Standard Specification and Test Methods for Metallic Medical Bone Screws. West Conshohocken, Pa.: ASTM International; 2014.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. An polymer-permeated graft for in vivo use in a subject comprising a tissue substantially free of cells, wherein the decellularized tissue is substantially free of water and is permeated with polymer, and wherein the polymer is substantially uniformly distributed in said tissue.

2. The graft of claim 1, wherein the graft comprises less than about 50% polymer.

3. The graft of claim 2, wherein the graft comprises about 0.1% to about 30% polymer.

4. The graft of claim 1, wherein the tissue comprises a dermal tissue and/or an epidermal tissue.

5. The graft of claim 1, wherein the tissue comprises an organ, a muscle, a ligament, a bone, a nipple, areola, a nipple attached to an areola, a lip, skin, a tendon, an aorta, a blood vessel.

6. The graft of claim 1, wherein the tissue substantially retains at least one matrix molecule.

7. The graft of claim 1, wherein the matrix molecule comprises a component of the extracellular matrix.

8. The graft of claim 1, wherein the matrix molecule comprises laminin, elastin, fibronectin, collagen, or a combination thereof.

9. The graft of claim 8, wherein the collagen comprises a Type I collagen, a Type III collagen, a Type IV collagen, a Type VI collagen, or a combination thereof.

10. The graft of claim 1, wherein the tissue is substantially free of skin, fat and/or fibrous tissue.

11. The graft of claim 1, wherein the polymer comprises a colored polymer.

12. The graft of claim 11, wherein the colored polymer comprises melanin, a dye, or a combination thereof.

13. The graft of claim 1, wherein the polymer comprises a natural polymer and/or a synthetic polymer.

14. The graft of claim 1, wherein the natural polymer comprises alginate or collagen.

15. The graft of claim 1, wherein the synthetic polymer comprises cyanoacrylate.

16. The graft of claim 1, wherein the polymer comprises a polymer comprising at least one viable cell, a polymer comprising at least one antibiotic, a biodegradable polymer, a non-biodegradable polymer, a polymer capable of cross-linking, or a combination thereof.

17. The graft of claim 1, wherein the biodegradable polymer comprises chitosan, collagen, alginate, cyanoacrylate, dermabond.

18. The graft of claim 1, wherein the non-biodegradable polymer comprises silicon, UHMWPE.

19. The graft of claim 1, wherein the graft further comprises viable cells, wherein the cells have been introduced into graft under conditions conducive to repopulate the tissue with the cells or progeny thereof.

20. The graft of claim 19, wherein the cells comprise exogenous cells, autologous cells, allogenic cells.

21. The graft of claim 19, wherein the viable cells comprise stromal cells, fibroblasts, endothelial cells, progenitor cells, stem cells, organ-specific cells, tissue-specific cells, keratinocytes, melanocytes, a nerve cell, or a combination thereof.

22. A method of making a polymer-permeated graft for in vivo use comprising:
obtaining a decellularized tissue;
optionally, fixing the decellularized tissue by submerging the decellularized tissue in a fixative for a period of time sufficient to fix the tissue;
replacing substantially all of the water within the tissue with a solvent by submerging the decellularized tissue in the solvent for a period of time and at a temperature sufficient to replace all or substantially all of the water within the tissue;
optionally, removing all or substantially all of the fat within the tissue by submerging the decellularized tissue in a solvent for a period of time and at a temperature sufficient to remove substantially all lipids;
permeating the tissue with a polymer by submerging the tissue in the polymer and subjecting the submerged tissue to vacuum for a period of time sufficient to permeate the tissue with the polymer;
optionally, cross-linking the polymer permeated within the tissue;
wherein the polymer-permeated tissue comprises the polymer substantially uniformly distributed in said tissue;
thereby providing a polymer-permeated graft for in vivo use.

23. The method of claim 22, wherein a chemical cross-linker has been admixed with the polymer prior to permeating the tissue.

24. The method of claim 22, further comprising decellularizing a tissue of cells of the epidermis and/or cells of the dermis, while substantially retaining at least one matrix molecule.

25. The method of claim 24, wherein the matrix molecule comprises laminin, fibronectin, elastin, collagen.

26. The method of claim 25, wherein the collagen comprises a Type I collagen, a Type III collagen, a Type IV collagen, a Type VI collagen, or a combination thereof.

27. The method of claim 22, further comprising repopulating the tissue with viable cells under conditions conducive to repopulate the tissue with the cells or progeny thereof.

28. The method of claim 27, wherein the repopulating occurs at about the same time as the permeating step.

29. The method of claim 27, wherein the repopulating occurs after the permeating step.

30. The method of claim 27, wherein the cells comprise exogenous cells, autologous cells, allogenic cells.

31. The method of claim 27, wherein the cells comprise keratinocytes, melanocytes, a nerve cell, or a combination thereof.

32. The method of claim 22, wherein the fixative comprises glutaraldehyde, genipin.

33. The method of claim 22, wherein the solvent comprises acetone, xylene, alcohol.

34. The method of claim 33, wherein the decellularized tissue is incubated in acetone at about −15° C. to 25° C. for a period of time.

35. The method of claim 22, wherein cross-linking comprises UV cross-linking, chemical cross-linking.

36. A method of grafting to a subject a polymer-impregnated graft, comprising obtaining the polymer-impregnated graft of claim 1 and implanting the polymer-permeated graft to a site on the subject; thereby grafting to a subject the polymer-permeated graft.

37. A method of treating a subject afflicted with POP, the method comprising:
obtaining a polymer-impregnated graft of claim 1; and
implanting the polymer-permeated graft to in the subject.

38. The method of claim 36, wherein the graft has been repopulated with viable cells.

39. The method of claim 37, wherein the cells comprise exogenous cells, autologous cells, allogenic cells.
